(19) United States
(12) Patent Application Publication
(10) Pub. No.: US 2026/0283956 A1
(43) Pub. Date: Sep. 24, 2026

(54) CATIONIC POLY(OXAZOLINE) LIPID CONJUGATES AND LIPID NANOPARTICLES AND POLYPLEXES INCLUDING SAME

(71) Applicant: Serina Therapeutics (AL), Inc., Hunstville, AL (US)

(72) Inventors: Wookhyun Kim, Madison, AL (US); Randall Moreadith, Huntsville, AL (US); Kunsang Yoon, Madison, AL (US); Kelley Smith, Madison, AL (US)

(73) Assignee: Serina Therapeutics (AL), Inc., Huntsville, AL (US)

(21) Appl. No.: 19/574,779

(22) Filed: Mar. 23, 2026

Related U.S. Application Data
(60) Provisional application No. 63/776,384, filed on Mar. 24, 2025.

(51) Int. Cl. A61K 9/1272; A61K 9/51; C12N 15/88

(52) U.S. Cl. CPC A61K 9/1272; A61K 9/5123; C12N 15/88

(57) ABSTRACT

Poly(oxazoline)-lipid conjugates with pendant cationic groups (cationic POZ-lipid) and lipid nanoparticles (LNPs) including cationic POZ-lipids used to facilitate delivery of an encapsulated payload. LNPs and polyplexes including cationic POZ-lipid and a nucleic acid payload such as, but not limited to, mRNA or modified mRNA are disclosed. Such LNPs have no immunogenicity or reduced immunogenicity as compared to a corresponding LNP containing an ionizable lipid.

### FIELD OF THE INVENTION

The present disclosure relates to cationic polyoxazoline (POZ) lipid conjugates and methods of synthesis. In addition, the present disclosure relates to lipid nanoparticles (LNPs) and polyplexes including cationic POZ-lipids and pharmaceutical compositions including such LNPs. LNPs and polyplexes incorporating oligonucleotides such as mRNA, DNA, saRNA and siRNA for delivery into living cells is also contemplated.

### BACKGROUND OF THE INVENTION

Nucleic-acid (particularly mRNA)-based vaccines offer advantages over other vaccine technologies. Such vaccines can be produced with reduced development time and costs by employing a common manufacturing platform based on lipid nanoparticles (LNPs). In fact, the utility of LNPs for delivering mRNA to cells has been demonstrated in vaccines for Covid-19 (or SARS-CoV-2) to address the global pandemic that led to over 7 million deaths worldwide. Given the extraordinary burden on health care systems resulting from this viral disease and given the apparent wide utility of cellular delivery of mRNA for a range of diseases from cancer to influenza, there has been a large amount of work on understanding and improving LNP delivery of RNA.

As shown in FIG. 1, the basic structure of a LNP is accomplished by four components—(1) a sterol lipid (to provide flexibility, e.g., cholesterol), (2) a helper lipid (to provide structural integrity, e.g., such as distearoylphosphatidylcholine (DSPC)), (3) a polymer lipid (to stabilize the lipid nanoparticles and prevent fusion with other nanoparticles, e.g., a PEG-lipid or a POZ-lipid) and (4) an ionizable lipid (which complexes with the oligonucleotide). Most commonly, these components are prepared in two separate phases: 1) an aqueous phase (containing the RNA or other oligonucleotide) in buffers at approximately pH 4-5, and 2) an organic phase containing the cholesterol/DSPC/polymer-lipid/ionizable lipid components in ethanol. By employing microfluidic techniques during which the flow rates of these two phases are precisely controlled, LNPs can be prepared of a desired size that facilitates the delivery of the nucleic acid payload to produce a therapeutic response.

In this aspect, the ionizable lipid has been shown to be a critical component of LNPs. However, preclinical studies have suggested the ionizable lipid is implicated in a host of undesirable side effects from enhanced reactogenicity in patients (adverse events) to abnormal blood coagulation, which include binding of PEG-lipid nanoparticles following immunization with the approved mRNA vaccines, activation of the complement cascade, induction of proinflammatory pathways and accelerated clearance of the LNP. Thus, while the nature of the ionizable lipid may have profound effects on delivery and expression of the payloads (for example MC3 works exceptionally well for delivery of siRNA but generally not well for delivery of mRNA), the field is in need of developing technology that reduces or eliminates the undesirable side effects upon repeat dose administration that may be associated with the ionizable lipid. More specifically, there is a need in the art for a non-immunogenic (or at least markedly reduced immunogenic) replacement for the ionizable lipid without compromising the efficacy of the LNP.

Synthetic cationic polymers are being used as non-viral vectors in gene therapy. In this aspect, long DNA chains are collapsed into nanoparticles of 50-300 nm in size by condensation with polyamines such as poly(L-lysine), spermidine, and spermine (Thomas, T. J., Heidar-Ali Tajmir-Riahi, and C. K. S. Pillai (2019). “Biodegradable Polymers for Gene Delivery” Molecules 24, no. 20: 3744. https://doi.org/10.3390/molecules24203744). However, highly positively charged polymers with primary amines have high zeta-potential values and may cause cytotoxicity.

Polymer chains with secondary amines have been also used, and these include the polyethyleneimine (PEI) linear and branched polymers. These polymers have been shown to have high transfection efficiencies. For example, U.S. Pat. No. 11,318,195 describes polyplexes that were made with PEI of 5000 to 25000 Da molecular weight and single stranded RNA with N/P ratios of 2 to 15. The toxicity of free and pure PEI on HEK-293 cells shows an IC₅₀ of 77 μM. To reduce these toxic effects, these polymers have been co-polymerized with inert polymers such as polyethylene glycol (PEG), polysaccharides, dextran, polycaprolactone and polyglutamic acid.

Co-polymers of poly(methyl oxazoline) and poly(decenyl oxazoline) or poly(butenyl oxazoline) have been discussed. See, e.g., Rinkenauer A C et al. (March 2015): A Cationic Poly(2-oxazoline) with High In Vitro Transfection Efficiency Identified by a Library Approach; Macromolecular Bioscience Vol. 15, Issue 3, Pages 414-425 (https://doi.org/10.1002/mabi.201400334). In this aspect, the “longer” decenyl or “shorter” butenyl side chains are attached to a thiol amine by photoaddition in order to create charged oxazoline polymers with thioether primary or tertiary amines. The polymer sizes are about 20,000 Da to 30,000 Da in molecular size and the polydispersity indices (PDI) are about 1.4. These polymers were condensed with plasmid pEGFP-N1 (4.7 kb) to make polyplexes 90 to 200 nm size and zeta-potential values from −4 to +40 mV. However, the polyplexes had very low transfection properties (less than 25 percent). Moreover, these polymers were not safe and were found to hemolyze erythrocytes.

In addition to the safety concerns with the cationic polymers and polyplexes discussed above, there have been no attempts to prepare LNPs from these polymers or polyplexes until U.S. Patent Publication No. 2025/0017857. The present disclosure provides an additional solution to the shortcomings of the prior art.

### SUMMARY OF THE INVENTION

The present disclosure relates to cationic polyoxazoline (POZ) lipid conjugates (cationic POZ-lipid) suitable as a replacement for the ionizable lipids that are used in lipid nanoparticles such as commercially available mRNA vaccines. In particular, the inventors have now made the surprising observation that LNPs made with ionizable/cationic POZ-lipid provide efficient and therapeutic delivery of RNA and DNA payloads.

In some embodiments, the ionizable/cationic POZ-lipid is cationic poly(oxazoline) lipid (cationic POZ-lipid). In this aspect, LNPs may be made in accordance with the present disclosure including a sterol lipid, a helper lipid, a polymer lipid, and a cationic POZ-lipid (cationic POZ-lipid LNP). In some embodiments, the polymer lipid may be POZ-lipid. For example, a cationic POZ-lipid LNP may be made with cholesterol, DSPC, POZ-lipid, and cationic POZ-lipid. Similarly, polyplexes may be made with a cationic POZ-lipid and payload.

The present disclosure also relates to a lipid nanoparticle including:
a cationic POZ-lipid;
a helper lipid;
a polymer lipid; and
a sterol lipid, wherein the cationic POZ-lipid is selected from the group consisting of one of the cationic POZ-lipid of Formulae I-II:

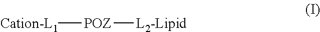

where L₁ includes a first linking group,
POZ includes a polyoxazoline polymer,
L₂ includes a second linking group, and
Lipid includes a non-charged lipid;

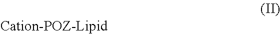

where POZ includes a polyoxazoline polymer, and Lipid includes a non-charged lipid.

In some embodiments, the lipid nanoparticle includes about 15 percent to about 60 percent cationic POZ-lipid, about 35 percent to about 70 percent sterol lipid, about 0.5 percent to about 10 percent polymer lipid, and about 5 percent to about 20 percent helper lipid. In other embodiments, the lipid nanoparticle includes about 20 percent to about 45 percent cationic POZ-lipid, about 40 percent to about 65 percent sterol lipid, about 0.5 percent to about 5 percent polymer lipid, and about 5 percent to about 15 percent helper lipid.

In still other embodiments, the lipid nanoparticle includes about 5 percent to about 60 percent cationic POZ-lipid, about 30 percent to about 85 percent sterol lipid, about 0.5 percent to about 10 percent polymer lipid, and about 5 percent to about 20 percent helper lipid. In other embodiments, the lipid nanoparticle includes about 10 percent to about 50 percent cationic POZ-lipid, about 35 percent to about 80 percent sterol lipid, about 0.5 percent to about 5 percent polymer lipid, and about 5 percent to about 15 percent helper lipid.

The present disclosure also relates to a cationic POZ-lipid of Formula I(a):

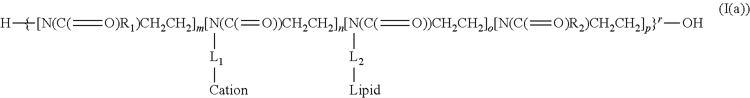

where R₁ is independently selected for each repeating unit from an unsubstituted or substituted alkyl,
m may range from 0 to 25,
L₁ includes a first linking group,
n may range from 1 to 4,
L₂ includes a second linking group,
o may range from 1 to 4,
R₂ is independently selected for each repeating unit from an unsubstituted or substituted alkynyl,
p may range from 0 to 3, and
r is random.

In some embodiments, R₁ is ethyl. In other embodiments, R₁ is propyl. In still other embodiments, R₂ is pentynyl. In yet other embodiments, n+o+p may range from 2 to 5.

The present disclosure also relates to a cationic POZ-lipid of Formula II(a):

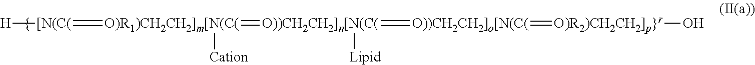

where R₁ is independently selected for each repeating unit from an unsubstituted or substituted alkyl,
m may range from 0 to 25,
n may range from 1 to 4,
o may range from 1 to 4,
R₂ is independently selected for each repeating unit from an unsubstituted or substituted alkynyl,
p may range from 0 to 3, and
r is random.

In some embodiment, R₁ is ethyl. In other embodiments, R₁ is propyl. In yet other embodiments, R₂ is pentynyl, propynyl, butynyl, or acetylenyl. In still other embodiments, n+o+p may range from 2 to 5.

The present disclosure also relates to a cationic POZ-lipid of Formula II(b):

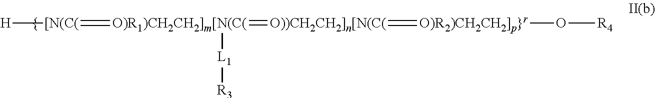

where R₁ is independently selected for each repeating unit from an unsubstituted or substituted alkyl,
R₃ is Cation,
m may range from 0 to 25,
L₁ includes a first linking group,
n may range from 1 to 5,
R₂ is independently selected for each repeating unit from an unsubstituted or substituted alkynyl,
p may range from 0 to 4,
r is random, and
R₄ is Lipid.

In some embodiments, R₁ is ethyl. In other embodiments, R₁ is propyl. In yet other embodiments, R₂ is pentynyl. In still other embodiments, n+p may range from 1 to 5.

The present disclosure also relates to a cationic POZ-lipid of Formula II(c):

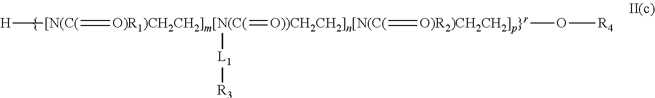

where R₁ is independently selected for each repeating unit from an unsubstituted or substituted alkyl,
R₃ is Lipid,
m may range from 0 to 25,
L₁ includes a first linking group,
n may range from 1 to 5,
R₂ is independently selected for each repeating unit from an unsubstituted or substituted alkynyl,
p may range from 0 to 4,
r is random, and
R₄ is Cation.

In some embodiments, R₁ is ethyl. In other embodiments, R₁ is propyl. In yet other embodiments, R₂ is pentynyl. In still other embodiments, n+p may range from 1 to 5.

A cationic POZ-lipid conjugate may also be formed by “clicking” on the pendant and esterification on the terminal of the polymer chain. In this aspect, the cationic POZ-lipid may be of Formula II(d):

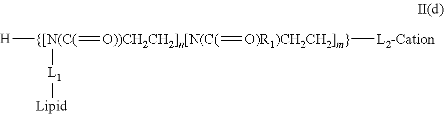

where R₁ is independently selected for each repeating unit from an unsubstituted or substituted alkyl,
n may range from 1 to 2,
L₁ includes a first linking group,
m may range from 1 to 25,
L₂ includes a second linking group.

In some aspects, R₁ is ethyl. In other aspects, R₁ is propyl. In yet other aspects, R₁ is pentynyl, propynyl, butynyl, or acetylenyl. In some aspects, n+m may range from 1 to 20. In other aspects, n+m may range from 1 to 10. In still other aspects, n+m may range from 2 to 6.

The present disclosure also relates to a cationic POZ-lipid of Formula II(e):

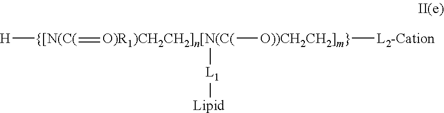

where R₁ is independently selected for each repeating unit from an unsubstituted or substituted alkyl,
n may range from 1 to 25
L₁ includes a first linking group,
m may range from 1 to 2,
L₂ includes a second linking group.

In some aspects, R₁ is ethyl. In other aspects, R₁ is propyl. In yet other aspects, R₁ is pentynyl, propynyl, butynyl, or acetylenyl. In some aspects, n+m may range from 1 to 20. In other aspects, n+m may range from 1 to 10. In still other aspects, n+m may range from 2 to 6.

The present disclosure also relates to lipid nanoparticle compositions formed with the cationic POZ-lipids of any of Formulae I(a), II(b), II(c), II(d), or II(e) a helper lipid, a polymer lipid, and, a sterol lipid.

### BRIEF DESCRIPTION OF THE DRAWINGS

Further features and advantages of the invention can be ascertained from the following detailed description that is provided in connection with the drawings described below:

FIG. 1 shows a conventional lipid nanoparticle;

FIG. 2 shows a lipid nanoparticle in accordance with an embodiment of the present disclosure;

FIGS. 3A-3D show the particle size of LNPs containing PPOZ-OH 2.6k 1p NMPCA-Lipid formed in accordance with embodiments of the present disclosure;

FIG. 4 shows the zeta potential of LNPs containing PPOZ-OH 2.6k 1p NMPCA-Lipid formed in accordance with embodiments of the present disclosure;

FIG. 5 shows positive gene targeting of LoxP-flanked target by CRE recombinase with LNPs formed in accordance with embodiments of the present disclosure;

FIGS. 6A-6B shows cell viability of saRNA LNPs formed in accordance with embodiments of the present disclosure;

FIG. 7 shows in vitro transfection of HEK293 cells with EGFP saRNA-LNPs containing PPOZ-OH 2.6k 1p NMPCA-Lipid formed in accordance with embodiments of the present disclosure;

FIG. 8 shows in vitro transfection of HEK293 cells with FLuc saRNA-LNPs containing PPOZ-OH 2.6k 1p NMPCA-Lipid formed in accordance with embodiments of the present disclosure;

FIGS. 9A-9F shows the particle size of LNPs containing DO-[PPOZ 500/PEOZ 700]-IG formed in accordance with embodiments of the present disclosure;

FIG. 10 shows the zeta potential of LNPs containing DO-[PPOZ 500/PEOZ 700]-IG formed in accordance with embodiments of the present disclosure;

FIG. 11 shows in vitro transfection of HEK293 cells with FLuc saRNA-LNPs containing DO-[PPOZ 500/PEOZ 700]-IG formed in accordance with embodiments of the present disclosure;

FIG. 12 shows cellular uptake of FITC-labeled RNA LNPs containing DO-[PPOZ 500/PEOZ 700]-NMP formed in accordance with embodiments of the present disclosure; and

FIGS. 13A-13B shows in vitro transfection of HEK293 cells with FLuc saRNA-LNPs containing DO-[PPOZ 500/PEOZ 700]-NMP formed in accordance with embodiments of the present disclosure.

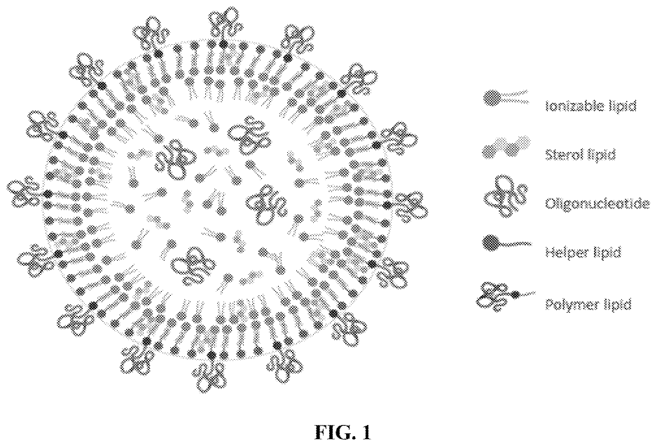

### DETAILED DESCRIPTION OF THE INVENTION

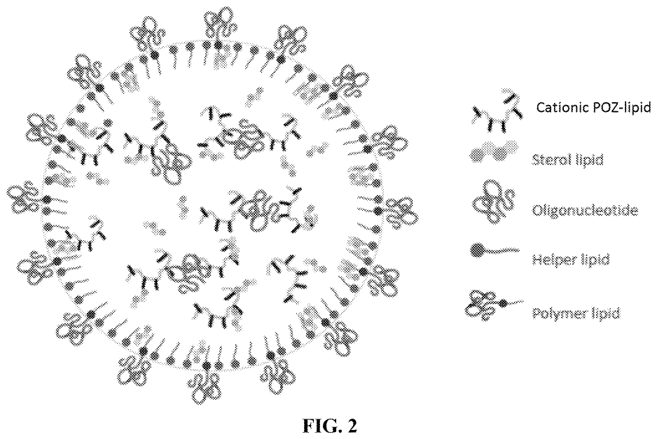

The present disclosure provides for cationic poly(oxazoline)-lipid conjugates (cationic POZ-lipid) suitable for replacement of the ionizable lipid in LNPs. In particular, a cationic POZ-lipid LNP may be made as shown in FIG. 2 and include a cationic POZ-lipid, a sterol lipid, a helper lipid, and a polymer lipid. The cationic POZ-lipid LNPs made in accordance with the present disclosure may be used to preferentially deliver an encapsulated payload, e.g., a nucleic acid payload including, but not limited to, mRNA or modified mRNA.

The cationic POZ-lipids and other components of the cationic POZ-lipid LNP are discussed in greater detail below.

### Definitions

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

The articles “a” and “an” are used herein to refer to one or to more than one (i.e., to at least one) of the grammatical object of the article. By way of example, “an element” means one element or more than one element.

The terms “about” and “approximately” shall generally mean an acceptable degree of error or variation for the quantity measured given the nature or precision of the measurements. Numerical quantities given in this description are approximate unless stated otherwise, meaning that the term “about” or “approximately” can be inferred when not expressly stated.

As used herein, the term “active” or “activated” when used in conjunction with a particular functional group refers to a functional group that reacts readily with an electrophile or a nucleophile on another molecule. This is in contrast to those groups that require catalysts or impractical reaction conditions in order to react (i.e., a “non-reactive” or “inert” group).

As used herein, the term “physiologically degradable” or “physiologically releasable” refers to a linkage containing a cleavable moiety. The terms degradable and releasable do not imply any particular mechanism by which the linker is cleaved.

As used herein, the term “link”, “linked” “linkage” or “linker” when used with respect to a POZ polymer, POZ conjugate, an agent, or compound described herein, or components thereof, refers to bonds that normally are formed as the result of a chemical reaction and typically are covalent linkages.

As used herein, the term “lipid nanoparticle” or “LNP” is used to encompass any of the many types of nanoparticles, including liposomes, that are formed by a lipid layer or layers surrounding a core containing a molecule to be released into the body. Liposomes generally have one or more contiguous lipid bilayers encapsulating an aqueous core. Other forms of liposome-like nanocarriers may have a lipid monolayer, or a non-contiguous bilayer, and may or may not have an aqueous core.

As used herein, the term “hydrophilic,” for example with reference to a hydrophilic group, refers to a compound or molecule, or a portion thereof, where the interaction with water is thermodynamically more favorable than interaction with oil or other hydrophobic solvents. A hydrophilic compound is able to dissolve in, or be dispersed in, water.

As used herein, the term “hydrophobic”, for example with reference to a hydrophobic portion, refers to a compound or molecule, or a portion thereof, where the interaction with water is thermodynamically less favorable than interaction with oil or other hydrophobic solvents. A hydrophobic compound is able to dissolve in, or be dispersed in, oil or other hydrophobic solvents.

As used herein, the term “inert” or “non-reactive” when used in conjunction with a particular functional group refers to a functional group that does not react readily with an electrophile or a nucleophile on another molecule and require catalysts or impractical reaction conditions in order to react.

As used herein, the term “pendent group” refers to a part of the POZ polymer that is attached to the POZ polymer.

As used herein, the term “pendent moiety” refers to a substituent that is linked to the POZ polymer portion via a linking group.

As used herein, the term “pharmaceutically acceptable” refers to a compound that is compatible with the other ingredients of a composition and not deleterious to the subject receiving the compound or composition. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “pharmaceutically acceptable form” is meant to include known forms of a compound or POZ conjugate that may be administered to a subject, including, but not limited to, solvates, hydrates, prodrugs, isomorphs, polymorphs, pseudomorphs, neutral forms and salt forms of a compound. In certain embodiments, the pharmaceutically acceptable form excludes prodrugs, isomorphs and/or pseudomorphs. In certain embodiments, the pharmaceutically acceptable form is limited to pharmaceutically acceptable salts, neutral forms, solvates and hydrates. In certain embodiments, the pharmaceutically acceptable form is limited to pharmaceutically acceptable salts and neutral forms. In certain embodiments, the pharmaceutically acceptable form is limited to pharmaceutically acceptable salts.

As used herein, the term “alkyl”, whether used alone or as part of a substituent group, is a term of art and refers to saturated aliphatic groups that optionally contain one or more heteroatoms (such as O, S or N) which may be optionally substituted, including straight-chain alkyl groups, branched-chain alkyl groups, cycloalkyl groups, alkyl substituted cycloalkyl groups, and cycloalkyl substituted alkyl groups. In certain embodiments, a straight-chain or branched-chain alkyl has about 30 or fewer carbon atoms in its backbone (e.g., C₁-C₃₀ for straight chain, C₃-C₃₀ for branched chain), and alternatively, about 20 or fewer, or 10 or fewer. In certain embodiments, the term “alkyl” refers to a C₁-C₁₀ straight-chain alkyl group or a C₁-C₃ straight-chain alkyl group. In certain embodiments, the term “alkyl” refers to a C₃-C₁₂ branched-chain alkyl group. In certain embodiments, the term “alkyl” refers to a C₃-C₈ branched-chain alkyl group. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl. In certain embodiments, the term “alkyl” refers to a C₁-C₁₀ straight-chain alkyl group that contains one or more heteroatoms in place of a carbon atom (such as O, S or N), wherein the heteroatom may be optionally substituted. In certain embodiments, the term “alkyl” refers to a C₁-C₁₀ straight-chain alkyl group that is substituted with up to 5 groups selected from the group consisting of OH, NH₂ and ═O.

As used herein, the term “alkenyl”, whether used alone or as part of a substituent group, is a term of art and refers to unsaturated aliphatic groups that optionally contain one or more heteroatoms (such as O, S or N) which may be optionally substituted, including, a straight or branched chain hydrocarbon radical containing from 2 to 30 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl. The unsaturated bond(s) of the alkenyl group can be located anywhere in the moiety and can have either the (Z) or the (E) configuration about the double bond(s).

As used herein, the term “alkynyl”, whether used alone or as part of a substituent group, is a term of art and refers to unsaturated aliphatic groups that optionally contain one or more heteroatoms (such as O, S or N) which may be optionally substituted, including, straight or branched chain hydrocarbon radical containing from 2 to 30 carbon atoms and containing at least one carbon-carbon triple bond. Representative examples of alkynyl include, but are not limited, to acetylenyl, 1-propynyl, 2-propynyl, 3-butynyl, 2-pentynyl, 4-pentynyl, and 1-butynyl.

As used herein, the term “substituted alkyl”, “substituted alkenyl”, and “substituted alkynyl” refers to alkyl, alkenyl and alkynyl groups as defined above in which one or more bonds to a carbon(s) or hydrogen(s) are replaced by a bond to non-hydrogen or non-carbon atoms such as, but not limited to, a halogen atom in halides such as F, Cl, Br, and I; and oxygen atom in groups such as carbonyl, carboxyl, hydroxyl groups, alkoxy groups, aryloxy groups, heterocyclyloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, enamines imines, oximes, hydrazones, heterocyclylamine, (alkyl)(heterocyclyl)-amine, (aryl)(heterocyclyl)amine, diheterocyclylamine, triazoles, and nitriles; a silicon atom in groups such as in trialkylsilyl groups, dialkylarylsilyl groups, alkyldiarylsilyl groups, and triarylsilyl groups; and other heteroatoms in various other groups. In a specific embodiment, a “polar alkyl”, “polar alkenyl”, and “polar alkynyl”, refers to alkyl, alkenyl, and alkynyl groups substituted with an atom that results in a polar covalent bond. In another specific embodiment, a “polar alkyl”, “polar alkenyl”, and “polar alkynyl” refers to C₁ to C₅ alkyl, alkenyl, and alkynyl, groups substituted with an atom that results in a polar covalent bond. In a specific embodiment, a “polar alkyl”, “polar alkenyl”, and “polar alkynyl”, refers to alkyl, alkenyl, alkynyl groups, such as C₁ to C₅ alkyl, alkenyl, and alkynyl groups, substituted with an —OH group and/or a —C(O)—OH group.

As used herein, the term “halo” or “halogen” whether used alone or as part of a substituent group, is a term of art and refers to —F, —Cl, —Br, or —I.

As used herein, the term “alkoxy”, whether used alone or as part of a substituent group, is a term of art and refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

As used herein, the term “aralkyl” or “arylalkyl”, whether used alone or as part of a substituent group, is a term of art and refers to an alkyl group substituted with an aryl group, wherein the moiety is appended to the parent molecule through the alkyl group. An arylalkyl group may be optionally substituted. A “substituted aralkyl” has the same meaning with respect to unsubstituted aralkyl groups that substituted aryl groups had with respect to unsubstituted aryl groups. However, a substituted aralkyl group also includes groups in which a carbon or hydrogen bond of the alkyl part of the group is replaced by a bond to a non-carbon or a non-hydrogen atom.

As used herein, the term “heteroaralkyl” or “heteroarylalkyl”, whether used alone or as part of a substituent group, is a term of art and refers to an alkyl group substituted with a heteroaryl group, wherein the moiety is appended to the parent molecular moiety through the alkyl group. A heteroarylalkyl may be optionally substituted. The term “substituted heteroarylalkyl” has the same meaning with respect to unsubstituted heteroarylalkyl groups that substituted aryl groups had with respect to unsubstituted aryl groups.

As used herein, the term “heterocyclylalkyl”, whether used alone or as part of a substituent group, is a term of art and refers to unsubstituted or substituted alkyl, alkenyl or alkynyl groups in which a hydrogen or carbon bond of the unsubstituted or substituted alkyl, alkenyl or alkynyl group is replaced with a bond to a heterocyclyl group. A heterocyclylalkyl may be optionally substituted. The term “substituted heterocyclylalkyl” has the same meaning with respect to unsubstituted heterocyclylalkyl groups that substituted aryl groups had with respect to unsubstituted aryl groups. However, a substituted heterocyclylalkyl group also includes groups in which a non-hydrogen atom is bonded to a heteroatom in the heterocyclyl group of the heterocyclylalkyl group such as, but not limited to, a nitrogen atom in the piperidine ring of a piperidinylalkyl group.

As used herein, the term “aryl”, whether used alone or as part of a substituent group, is a term of art and refers to includes monocyclic, bicyclic and polycyclic aromatic hydrocarbon groups, for example, benzene, naphthalene, anthracene, and pyrene. The aromatic ring may be substituted at one or more ring positions with one or more substituents, such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “aryl” also includes polycyclic ring systems having two or more cyclic rings in which two or more carbons are common to two adjoining rings (the rings are “fused rings”) wherein at least one of the rings is an aromatic hydrocarbon, e.g., the other cyclic rings may be cycloalkyls, cycloalkenyls, cycloalkynyls, aryls, heteroaryls, and/or heterocyclyls. In certain embodiments, the term “aryl” refers to a phenyl group. The aryl group may be optionally substituted.

As used herein, the term “cycloalkyl”, whether used alone or as part of a substituent group, is a term of art and refers to a saturated carbocyclic group containing from three to six ring carbon atoms, wherein such ring may optionally be substituted with a substituted or unsubstituted alkyl group or a substituent as described for a substituted alkyl group. Exemplary cycloalkyl groups include, but are not limited to, cyclopropyl, cyclobutyl, cyclopentyl, cyclohexyl, 2-methylcyclobutyl and 4-ethylcyclohexyl.

As used herein, the term “heteroaryl”, whether used alone or as part of a substituent group, is a term of art and refers to a monocyclic, bicyclic, and polycyclic aromatic group having 3 to 30 total atoms including one or more heteroatoms such as nitrogen, oxygen, or sulfur in the ring structure. Exemplary heteroaryl groups include azaindolyl, benzo(b)thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl, and the like. The “heteroaryl” may be substituted at one or more ring positions with one or more substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “heteroaryl” also includes polycyclic ring systems having two or more cyclic rings in which two or more carbons are common to two adjoining rings (the rings are “fused rings”) wherein at least one of the rings is an aromatic group having one or more heteroatoms in the ring structure, e.g., the other cyclic rings may be cycloalkyls, cycloalkenyls, cycloalkynyls, aryls, heteroaryls, and/or heterocyclyls.

As used herein, the term “heterocyclyl”, whether used alone or as part of a substituent group, is a term of art and refers to a radical of a non-aromatic ring system, including, but not limited to, monocyclic, bicyclic, and tricyclic rings, which can be completely saturated or which can contain one or more units of unsaturation, for the avoidance of doubt, the degree of unsaturation does not result in an aromatic ring system, and having 3 to 15 atoms including at least one heteroatom, such as nitrogen, oxygen, or sulfur. For purposes of exemplification, which should not be construed as limiting the scope of this invention, the following are examples of heterocyclic rings: aziridinyl, azirinyl, oxiranyl, thiiranyl, thiirenyl, dioxiranyl, diazirinyl, diazepanyl, 1,3-dioxanyl, 1,3-dioxolanyl, 1,3-dithiolanyl, 1,3-dithianyl, imidazolidinyl, isothiazolinyl, isothiazolidinyl, isoxazolinyl, isoxazolidinyl, azetyl, oxetanyl, oxetyl, thietanyl, thietyl, diazetidinyl, dioxetanyl, dioxetenyl, dithietanyl, dithietyl, dioxalanyl, oxazolyl, thiazolyl, triazinyl, isothiazolyl, isoxazolyl, azepines, azetidinyl, morpholinyl, oxadiazolinyl, oxadiazolidinyl, oxazolinyl, oxazolidinyl, oxopiperidinyl, oxopyrrolidinyl, piperazinyl, piperidinyl, pyranyl, pyrazolinyl, pyrazolidinyl, pyrrolinyl, pyrrolidinyl, quinuclidinyl, thiomorpholinyl, tetrahydropyranyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. A heterocyclyl group may be substituted at one or more ring positions with one or more substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like.

As used herein, the terms “treatment”, “treat”, and “treating” refers a course of action (such as administering a conjugate as described herein or pharmaceutical composition comprising a conjugate as described herein) so as to prevent, eliminate, or reduce a symptom, aspect, or characteristics of a disease or condition. Such treating need not be absolute to be useful. In one embodiment, treatment includes a course of action that is initiated concurrently with or after the onset of a symptom, aspect, or characteristics of a disease or condition. In another embodiment, treatment includes a course of action that is initiated before the onset of a symptom, aspect, or characteristics of a disease or condition.

As used herein, the term “in need of treatment” refers to a judgment made by a caregiver that a patient requires or will benefit from treatment. This judgment is made based on a variety of factors that are in the realm of a caregiver's expertise, but that includes the knowledge that the patient is ill, or will be ill, as the result of a disease or condition that is treatable by a method or compound of the disclosure.

As used herein, the terms “individual”, “subject”, or “patient” refers to any animal, including mammals, such as mice, rats, other rodents, rabbits, dogs, cats, swine, cattle, sheep, horses, or primates, and humans. The terms may specify male or female or both, or exclude male or female. In a preferred embodiment, the terms “individual”, “subject”, or “patient” refers to a human.

As used herein, the term “therapeutically effective amount” refers to an amount of a conjugate, either alone or as a part of a pharmaceutical composition, that is capable of having any detectable, positive effect on any symptom, aspect, or characteristics of a disease or condition. Such effect need not be absolute to be beneficial.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, fragmentation, decomposition, cyclization, elimination, or other reaction.

It will be understood that when a group is specified as a part of a compound, the substitution of the group may be adjusted to accommodate the particular bonds. For example, when an alkyl group is joined to two other groups, the alkyl group is considered an alkylene group.

The term “substituted” is also contemplated to include all permissible substituents of organic compounds. In a broad aspect, the permissible substituents include acyclic and cyclic, branched and unbranched substituents, carbocyclic and heterocyclyl, aromatic and nonaromatic substituents of organic compounds. Illustrative substituents include, for example, those described herein above. For purposes of this disclosure, the heteroatoms, such as oxygen or nitrogen, may have hydrogen substituents and/or any permissible substituents of organic compounds described herein which satisfy the valences of the heteroatoms. Exemplary substitutions include, but are not limited to, hydroxy, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. This invention is not intended to be limited in any manner by the permissible substituents of organic compounds.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference). Unless otherwise defined, all technical and scientific terms used herein have the same meaning as commonly understood by one of ordinary skill in the art to which this invention pertains.

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of conjugate. As another example, the salt may comprise less than one inorganic or organic acid molecule per molecule of base, such as two molecules of conjugate per inorganic or organic acid molecule.

The terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, iso osmotic, cryo-preservatives, lubricating, flavoring, and coloring agents may be used. Other examples of suitable pharmaceutical carriers are described in Remington's Science and Practice of Pharmacy (23^{rd} edition, ISBN 9780128200070) and Handbook of Pharmaceutical Excipients (8^{th} edition, 978-0-85-711271-2), each herein incorporated by reference in their entirety.

As used herein, the term “target molecule” refers to any molecule having a therapeutic or diagnostic application or a targeting function, or a vehicle with which a compound is administered or formulated for administration, wherein the target molecule is capable of forming a linkage with an active functional group on a POZ polymer or a POZ derivative of the present disclosure, including, but not limited to, a therapeutic agent (such as but not limited to a drug), a diagnostic agent, a targeting agent, an organic small molecule, an oligonucleotide, a polypeptide, an antibody, an antibody fragment, a protein, a carbohydrate such as heparin or hyaluronic acid, or a lipid such as a glycerolipid, glycolipid, or phospholipid.

As used herein, “lipid” or “lipid portion” means (i) an organic compound that includes an ester of fatty acid or a derivative thereof and is characterized by being insoluble in water, but soluble in many organic solvents, and includes, but is not limited to, simple lipids such as fats, oils, and waxes, compound lipids such as phospholipids, glycolipids, cationic lipids, non-cationic lipids, neutral lipids, and anionic lipids, and derived lipids such as steroids, as well as (ii) an organic compound that does not include an ester of fatty acid, but mimics such an organic compound through its amphipathic character, i.e., it possesses both hydrophobic and hydrophilic portions, and, thus, is able to aggregate in a specific manner to form layers, vesicles and LNPs in aqueous environments.

As used herein, “small interfering RNA (siRNA)” means a class of double-stranded RNA molecules, 16-40 nucleotides in length, that are involved in the RNA interference (RNAi) pathway, where it interferes with the expression of a specific gene. In addition to their role in the RNAi pathway, siRNAs also act in RNAi-related pathways.

As used herein, “sgRNA” means a class of guide RNA molecules that are involved in CRISPR-Cas9 genome editing, where the sgRNA provides a template for precise genome editing to minimize “off-target” editing and maximize “on-target” editing.

As used herein, “saRNA” means a class of RNA molecules that are “self-amplifying” or “self-replicating” due to the nature of the encoded protein, typically a replicase, that result in multiple copies of the RNA.

As used herein, “RNA” means a molecule comprising at least one ribonucleotide residue, including siRNA, antisense RNA, single stranded RNA, microRNA, mRNA, noncoding RNA, self-amplifying RNA, sgRNA, gRNA and multivalent RNA. “Ribonucleotide” means a nucleotide with a hydroxyl group at the 2′ position of a β-D-ribo-furanose moiety, and includes but is not limited to, modified ribonucleotides The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution, and/or alteration of one or more nucleotides.

### Cationic POZ-Lipid

Without being bound by any particular theory, it is believed that cationic POZ-lipid is a suitable replacement for an ionizable lipid in a stable LNP. As such, without being bound by any particular theory, efficient and therapeutic delivery of RNA and DNA payloads is possible with LNPs made with cationic POZ-lipid (and without the conventionally employed ionizable lipid).

For example, it has now been discovered that clicking a lipid to a cationic poly(oxazoline) (cationic POZ) and substituting the conventionally used ionizable lipid makes a stable LNP that demonstrates the characteristic dependence on flow-rate ratios when incorporating an oligonucleotide for therapeutic delivery. In this aspect, LNPs prepared with microfluidic techniques produce large particles at very low flow rates, and a limit-size LNP that is independent of flow rate at slightly higher flow rates. This high efficiency manufacture of LNPs without any ionizable lipid components is not only surprising and unexpected, but also may have profound implications on a range of RNA and DNA therapeutics including vaccines, cancer immunotherapy and gene therapy. Indeed, the cationic POZ-lipid LNPs of the present disclosure may have no immunogenicity, or reduced immunogenicity, as compared to corresponding LNPs containing ionizable lipids and PEG-lipids and, as such, may provide a safer method of delivering the payload.

In some embodiments, the cationic POZ-lipid may include lipid and cation pendent groups. In this aspect, the cationic POZ-lipid may be of Formula I:

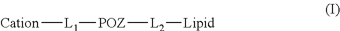

where L₁ includes a first linking group,
POZ includes a polyoxazoline polymer,
L₂ includes a second linking group, and
Lipid includes a non-charged lipid.

In some aspects, POZ may be a poly(ethyloxazoline) polymer. In other aspects, POZ may be poly(propyloxazoline) polymer. The cationic POZ-lipid may be any of the cationic POZ-lipids synthesized in the examples. In some embodiments, the cation and lipid may be conjugated by “click” chemistry. In some aspects, the first and/or second linking groups are physiologically degradable. In other aspects, the first and/or second linking groups are stable. The first and/or second linking groups may include esters, triazole-alkyl esters, carboxylate esters, carbonate esters, carbamates, amides, and combinations thereof.

One example of Formula I is shown below in Formula I(a):

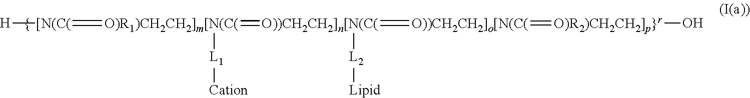

where R₁ is independently selected for each repeating unit from an unsubstituted or substituted alkyl,
m may range from 0 to 25,
L₁ includes a first linking group,
n may range from 1 to 4,
L₂ includes a second linking group,
o may range from 1 to 4,
R₂ is independently selected for each repeating unit from an unsubstituted or substituted alkynyl,
p may range from 0 to 3, and
r is random.

In some aspects, R₁ is ethyl. In other aspects, R₁ is propyl. In yet other aspects, R₂ is pentynyl, propynyl, butynyl, or acetylenyl. For example, R₂ may be 2-pentynyl or 4-pentynyl. In still other aspects, n+o+p may range from 2 to 5.

In other embodiments, the cationic POZ-lipid includes the lipid and cation without linking groups. In this aspect, the cationic POZ-lipid may be of Formula II:

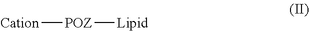

where POZ includes a polyoxazoline polymer, and Lipid includes a non-charged lipid. One example of Formula II is shown below in Formula II(a):

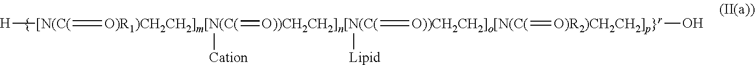

where R₁ is independently selected for each repeating unit from an unsubstituted or substituted alkyl,
m may range from 0 to 25,
n may range from 1 to 4,
o may range from 1 to 4,
R₂ is independently selected for each repeating unit from an unsubstituted or substituted alkynyl,
p may range from 0 to 3, and
r is random.

In some aspects, R₁ is ethyl. In other aspects, R₁ is propyl. In yet other aspects, R₂ is pentynyl, propynyl, butynyl, or acetylenyl. For example, R₂ may be 2-pentynyl or 4-pentynyl. In still other aspects, n+o+p may range from 2 to 5.

A cationic POZ-lipid conjugate may also be formed by “clicking” on the pendant and esterification on the terminal of the polymer chain. In this aspect, the cationic POZ-lipid may be of Formula II(b):

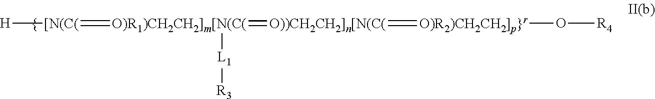

where R₁ is independently selected for each repeating unit from an unsubstituted or substituted alkyl,
R₃ is Cation,
m may range from 0 to 25,
L₁ includes a first linking group,
n may range from 1 to 5,
R₂ is independently selected for each repeating unit from an unsubstituted or substituted alkynyl,
p may range from 0 to 4,
r is random, and
R₄ is Lipid.

In some aspects, R₁ is ethyl. In other aspects, R₁ is propyl. In yet other aspects, R₂ is pentynyl, propynyl, butynyl, or acetylenyl. For example, R₂ may be 2-pentynyl or 4-pentynyl. In still other aspects, n+p may range from 1 to 5.

In other aspects, the cationic POZ-lipid may be of Formula II(c):

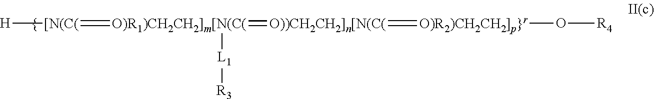

where R₁ is independently selected for each repeating unit from an unsubstituted or substituted alkyl,
R₃ is Lipid,
m may range from 0 to 25,
L₁ includes a first linking group,
n may range from 1 to 5,
R₂ is independently selected for each repeating unit from an unsubstituted or substituted alkynyl,
p may range from 0 to 4,
r is random, and
R₄ is Cation.

In some aspects, R₁ is ethyl. In other aspects, R₁ is propyl. In yet other aspects, R₂ is pentynyl, propynyl, butynyl, or acetylenyl. For example, R₂ may be 2-pentynyl or 4-pentynyl. In still other aspects, n+p may range from 1 to 5.

A cationic POZ-lipid conjugate may also be formed by “clicking” on the pendant and esterification on the terminal of the polymer chain. In this aspect, the cationic POZ-lipid may be of Formula II(d):

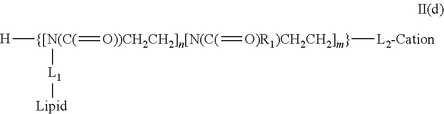

where R₁ is independently selected for each repeating unit from an unsubstituted or substituted alkyl,
n may range from 1 to 2,
L₁ includes a first linking group,
m may range from 1 to 425
L₂ includes a second linking group.

In some aspects, R₁ is ethyl. In other aspects, R₁ is propyl. In yet other aspects, R₁ is pentynyl, propynyl, butynyl, or acetylenyl. In some aspects, n+m may range from 1 to 20. In other aspects, n+m may range from 1 to 10. In still other aspects, n+m may range from 2 to 6.

The present disclosure also relates to a cationic POZ-lipid of Formula II(e):

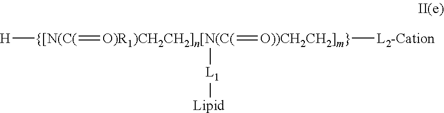

where R₁ is independently selected for each repeating unit from an unsubstituted or substituted alkyl,
n may range from 1 to 25,
L₁ includes a first linking group,
m may range from 1 to 2,
L₂ includes a second linking group.

In some aspects, R₁ is ethyl. In other aspects, R₁ is propyl. In yet other aspects, R₁ is pentynyl, propynyl, butynyl, or acetylenyl. In some aspects, n+m may range from 1 to 20. In other aspects, n+m may range from 1 to 10. In still other aspects, n+m may range from 2 to 6.

### Sterol Lipid

As briefly discussed above, cationic POZ-lipid LNPs made in accordance with the present disclosure include a sterol lipid for stability. A suitable sterol lipid for use in accordance with the present disclosure is cholesterol. Another suitable sterol lipid for use in accordance with the present disclosure is phytosterol.

### Helper Lipid

Cationic POZ-lipid LNPs made in accordance with the present disclosure include a helper lipid to provide structural support and facilitate endocytosis. Helper lipids refer to amphipathic lipids that have hydrophobic and polar head group moieties, and which can form spontaneously into bilayer vesicles in water, as exemplified by phospholipids, or are stably incorporated into lipid bilayers, with the hydrophobic moiety in contact with the interior, hydrophobic region of the bilayer membrane, and the polar head group moiety oriented toward the exterior, polar surface of the membrane. Such helper lipids typically include one or two hydrophobic acyl hydrocarbon chains or a steroid group and may contain a chemically reactive group, such as an amine, acid, ester, aldehyde or alcohol, at the polar head group. Non-limiting examples suitable for use in accordance with the present disclosure include phospholipids, such as phosphatidyl choline (PC), phosphatidyl ethanolamine (PE), phosphatidic acid (PA), phosphatidyl inositol (PI), phosphatidyl glycerol (PG), and sphingomyelin (SM), where the two hydrocarbon chains are typically between about 14-22 carbon atoms in length and have varying degrees of unsaturation. Other suitable helper lipids include, but are limited to, glycolipids, such as cerebrosides and gangliosides. In one aspect, the helper lipid is at least one of 1,2-dioleoyl-sn-glycero-3-phosphoethanolamine (DOPE), 1,2-distearoyl-sn-glycero-3-phosphocholine (DSPC), POPE (1-palmitoyl-2-oleoylsn-glycero-3-phosphoethanolamine), 1,2-distearoyl-sn-glycero-3-phospho-(1′-rac-glycerol) (DSPG), 1,2-dioleoyl-sn-glycero-3-phospho-(1′-rac-glycerol) (DOPG), 1′,3′-bis[1,2-distearoyl-sn-glycero-3-phospho]-glycerol (18:0 Cardiolipin), 1′,3′-bis[1,2-dioleoyl-sn-glycero-3-phospho]-glycerol (18:1 Cardiolipin).

### Polymer Lipid

In some embodiments, the polymer lipid may be a POZ-lipid of Formula VIII:

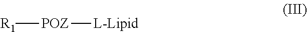

wherein R₁ includes an initiating group, POZ comprises poly(ethyloxazoline), L includes a physiologically degradable linking group, and Lipid includes a non-charged lipid including at least one hydrophobic moiety. In other embodiments, POZ includes [N(COR₂)CH₂CH₂]ₙ, where R₂ is ethyl. In still other embodiments, R₁ includes a hydrogen, a substituted or unsubstituted alkyl, an alkyne-substituted alkyl, a triazole with attached carboxylic acid, or a substituted or unsubstituted aralkyl group. In yet other embodiments, L includes ethers, esters, carboxylate esters, carbonate esters, carbamates, amines, amides, urethanes, disulfides, and combinations thereof. In still other embodiments, Lipid includes two hydrophobic moieties. In other embodiments, Lipid includes phospholipid, a glycerolipid, a di-alkyl acetamide, or a combination thereof. In yet other embodiments, Lipid includes 1,2-dimyristoyl-sn-glycerol, 1,2-dilauroyl-sn-glycerol, or a combination thereof.

In other embodiments, the polymer lipid may be a POZ-lipid of Formula IV:

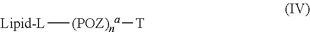

wherein Lipid includes a non-charged lipid including at least one hydrophobic moiety, L includes a physiologically degradable linking group, POZ includes a polyoxazoline polymer of the structure [N(COR₂)CH₂CH₂], wherein R₂ is ethyl, n ranges from 1 to 1,000, a is ran, which indicates a random copolymer, or block, which indicates a block copolymer, and T includes a terminating group.

In some embodiments, L includes ethers, esters, carboxylate esters, carbonate esters, carbamates, amines, amides, urethanes, disulfides, and combinations thereof. In other embodiments, L includes a triazole.

In yet other embodiments, T includes Z—B-Q, and wherein Z includes S, O, or N, B is an optional linking group, and Q is a terminating nucleophile or portion thereof. In still other embodiments, Lipid includes two hydrophobic moieties. In other embodiments, Lipid includes a phospholipid, a glycerolipid, a di-alkyl acetamide, or a combination thereof. In still other embodiments, Lipid includes 1,2-dimyristoyl-sn-glycerol, 1,2-dilauroyl-sn-glycerol, or a combination thereof.

In still other embodiments, the polymer lipid may be a POZ-lipid of Formula V:

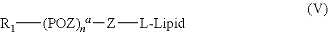

wherein R₁ includes an initiating group; POZ comprises a polyoxazoline polymer of the structure [N(COR₂)CH₂CH₂], wherein R₂ is ethyl; n ranges from 1 to 1,000; a is ran, which indicates a random copolymer, or block, which indicates a block copolymer; Z comprises S, O, or N; L includes a physiologically degradable linking group, and Lipid includes a non-charged lipid comprising at least one hydrophobic group.

In some embodiments, L includes ethers, esters, carboxylate esters, carbonate esters, carbamates, amines, amides, urethanes, disulfides, and combinations thereof. In other embodiments, Lipid includes two hydrophobic moieties. In still other embodiments, Lipid includes a phospholipid, a glycerolipid, a di-alkyl acetamide, or a combination thereof. In yet other embodiments, Lipid includes 1,2-dimyristoyl-sn-glycerol, 1,2-dilauroyl-sn-glycerol, or a combination thereof. In still other embodiments, R₁ includes a hydrogen, or a substituted or unsubstituted alkyl, and n ranges from 15 to 35.

In yet other embodiments, the polymer lipid may be a POZ-lipid of Formula VI:

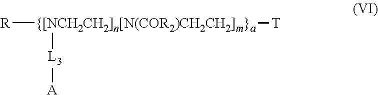

wherein R₁ includes an initiating group, L₃ includes a physiologically degradable linking group, Lipid includes a non-charged lipid comprising at least one hydrophobic moiety, n ranges from 1 to 5, R₂ is independently selected for each repeating unit from an unsubstituted or substituted alkyl, alkenyl, aralkyl, heterocyclylalkyl, or active functional group, m ranges from 1 to 100, a is ran, which indicates a random copolymer, or block, which indicates a block copolymer, and T includes a terminating group.

In some embodiments, L₃ includes esters, carboxylate esters, carbonate esters, carbamates, amides, and combinations thereof. In other embodiments, L₃ includes a triazole. In still other embodiments, T includes Z—B-Q, and wherein Z includes S, O, or N, B is an optional linking group, and Q is a terminating nucleophile or portion thereof. In yet other embodiments, Lipid includes two hydrophobic moieties. In still other embodiments, Lipid includes a phospholipid, a glycerolipid, a di-alkyl acetamide, or a combination thereof. In yet other embodiments, Lipid includes 1,2-dimyristoyl-sn-glycerol, 1,2-dilauroyl-sn-glycerol, or a combination thereof.

More specific embodiments and examples of suitable POZ lipids for use as the polymer lipid in a cationic POZ-lipid LNP made in accordance with the present disclosure are provided in U.S. Provisional Patent Application No. 63/440,210, the entire disclosure of which is incorporated by reference herein.

### Payload

As briefly discussed above, cationic POZ-lipid LNPs formed in accordance with the present disclosure may also include a payload. In this aspect, the payload may be an oligonucleotide, protein, or a combination thereof.

In one embodiment, the oligonucleotide comprises DNA, siRNA, self-replicating mRNA (saRNA), mRNA comprised of modified nucleosides, and mRNA comprised of naturally occurring nucleosides. In one aspect, the oligonucleotide is DNA. In another aspect, the oligonucleotide is siRNA. In still another aspect, the oligonucleotide is saRNA, mRNA comprised of modified nucleosides, or mRNA comprised of naturally occurring nucleosides. In still another aspect, the oligonucleotide is a sgRNA used in genome editing.

The oligonucleotide can be encapsulated into the LNP with high efficiency. In one embodiment, the oligonucleotide is encapsulated into the LNP with an efficiency of at least 90 percent. In another embodiment, the oligonucleotide is encapsulated into the LNP with an efficiency of about 90 to about 99 percent. In still another embodiment, the oligonucleotide is encapsulated into the LNP with an efficiency of about 90 to about 95 percent. In yet another embodiment, the oligonucleotide is encapsulated into the LNP with an efficiency of greater than about 95 percent.

Similarly, polyplexes may be made with the oligonucleotide and POZ polymer with high efficiency. In some embodiments, the oligonucleotide is incorporated into a polyplex with an efficiency of at least 90 percent, about 90 to about 99 percent, about 90 to about 95 percent, or greater than about 95 percent.

### Properties of Cationic POZ-Lipid LNP

The particle size of cationic POZ-lipid LNPs made in accordance with the present disclosure can vary. In one embodiment, cationic POZ-lipid LNPs formed in accordance with the present disclosure are amphiphilic spherical vesicles formed by one or more lipid bilayers enveloping an aqueous core with size ranging from about 10 nm to about 10 microns. In another embodiment, a cationic POZ-lipid LNP formed in accordance with the present disclosure has a particle size of about 25 nm to about 8 microns. In yet another embodiment, a cationic POZ-lipids LNP formed in accordance with the present disclosure has a particle size of about 30 nm to about 5 microns. In this aspect, the particle size of the cationic POZ-lipid LNP may be between about 20 nm to about 3 microns. In another embodiment, the cationic POZ-lipid LNP may be between about 10 nm and about 1000 nm, between about 25 nm and about 500 nm, between about 35 nm and about 300 nm, between about 40 nm and about 250 nm, or between about 45 nm and about 200 nm. In some embodiments, the cationic POZ-lipid LNPs may be between about 80 nm and about 110 nm. In other embodiments, the cationic POZ-lipid LNPs may be between about 85 nm and about 105 nm. Methods of size fractionation are disclosed herein. However, in certain aspects, size fractionation is not required.

### Methods of Preparing the Cationic POZ-Lipid LNP and Polyplexes

The cationic POZ-lipid LNP composition of the present disclosure may be prepared by a variety of methods. In one embodiment, the liposomes are prepared by the reverse-phase evaporation method (Szoka et al. PNAS 1978 vol. 75, 4194-4198; Smirnov et al., Byulleten' Éksperimental'noi Biologii i Meditsiny, 1984, Vol. 98, pp. 249-252; U.S. Pat. No. 4,235,871). In this method, an organic solution of liposome-forming lipids, which may include the polymer lipid, either with or without a linked target molecule, is mixed with a smaller volume of an aqueous medium, and the mixture is dispersed to form a water-in-oil emulsion, preferably using pyrogen-free components. The target molecule to be delivered is added either to the lipid solution, in the case of a lipophilic target molecule, or to the aqueous medium, in the case of a water-soluble target molecule. The lipid solvent is removed by evaporation and the resulting gel is converted to liposomes. The reverse phase evaporation vesicles (REVs) have typical average sizes between about 0.2-0.4 microns and are predominantly oligolamellar, that is, contain one or a few lipid bilayer shells. The REVs may be readily sized, as discussed below, by extrusion to give oligolamellar vesicles having a selected size preferably between about 0.05 to 0.2 microns.

In addition, multilamellar vesicles (MLVs) can be created. In this method, a mixture of liposome-forming lipids, which may include the polymer lipid, either with or without a linked target molecule, as described herein are dissolved in a suitable solvent is evaporated in a vessel to form a thin film. The thin film is then covered by an aqueous medium. The lipid film hydrates to form MLVs. MLVs generally exhibit sizes between about 0.1 to 10 microns. MLVs may be sized down to a desired size range by extrusion and other method described herein.

One effective sizing method for REVs and MLVs involves extruding an aqueous suspension of the liposomes through a polycarbonate membrane having a selected uniform pore size, typically 0.05, 0.08, 0.1, 0.2, or 0.4 microns (Szoka et al. PNAS 1978 vol. 75, 4194-4198). The pore size of the membrane corresponds roughly to the largest sizes of liposomes produced by extrusion through that membrane, particularly where the preparation is extruded two or more times through the same membrane. Process for sizing MLVs of larger sizes is provided by Zhu et al. (PLoS One. 2009; 4(4):e5009. Epub 2009 Apr. 6).

When small particle sizes are desired, the REV or MLV preparations can be treated to produce small unilamellar vesicles (SUVs) that are characterized by sizes in the 0.04-0.08 micron range. Such particles may be useful in targeting tumor tissue or lung tissue where the particles may be absorbed through capillary walls (particles larger than 0.1 microns may not be absorbed).

The cationic POZ-lipid LNP may be treated to remove extraneous components prior to use. For example, if surfactants are used as discussed above, the excess surfactants may be removed prior to use. In addition, where a payload, such as an oligonucleotide discussed above, is entrapped in the cationic POZ-lipid LNP composition, excess or non-entrapped payload may be removed prior to use. Separation techniques to accomplish this task are known in the art and the particular method selected may depend on the nature of the component to be removed. Suitable methods include, but are not limited to, centrifugation, dialysis and molecular-sieve chromatography. The composition can be sterilized by filtration through a conventional 0.22 micron filter.

Cationic POZ-lipid LNPs can be prepared by the traditional method that involves the hydration of a lipid film containing polymer lipid, cationic POZ-lipid, helper lipids, and cholesterol. This process involves the dissolution of these materials in organic solvent such as chloroform or dichloromethane and then evaporating the solvent to produce a thin film. The film is then hydrated with an aqueous buffer containing the drug or nucleic acid to passively encapsulate the payload. LNPs of heterogeneous particles with a low encapsulation are normally formed, which requires size reduction by extrusion or sonication.

Another suitable technique uses rapid mixing with a microfluidizer. Lipid stock solutions are prepared by dissolving the lipids in an organic solvent, such as ethanol. Aqueous stock solutions contain the nucleic acid dissolved in a buffer solution of known pH, ionic strength and buffer capacity. The two stock solutions are passed through a micromixer at a predetermined rate to allow for the cationic lipid to interact with the negatively charged nucleic acid, resulting in higher encapsulation efficiencies (i.e., >90 percent) and homogeneous size distribution. The aqueous-to-organic solvent ratios during the mixing process is important. The organic solvent is removed by dialysis, tangential flow filtration or centrifugation or other technique. LNPs of defined sizes are produced by controlling the microfluidic operating parameters, resulting in LNPs of low polydispersity and uniform particle size. The average particle diameter (<100 nm), polydispersity (<0.40 and, more particularly, <0.20), and zeta potential of the LNPs are three methods used to characterize the preparation.

The ratio of cationic POZ-lipid to polymer lipid to cholesterol can be varied in order to allow for optimal size, high payload release and transfection, and improved stability of the hydrated formulation. In addition, the lipid employed in the cationic-POZ-lipid may affect the mol percent of the components of the LNP. For example, when the lipid in the cationic-POZ-lipid is DPA, the cationic POZ-lipid may be included in the LNP in an amount of about 10 to about 25 mol percent of the LNP. And, when the lipid in the cationic is TOE, the cationic POZ-lipid may be included in the LNP in an amount of about 15 to about 25 mol percent of the LNP. But, when the lipid in the cationic is DO, the cationic POZ-lipid may be included in the LNP in an amount of about 10 to about 20 mol percent of the LNP.

Likewise, the POZ employed in the cationic-POZ-lipid may affect the mol percent of the components of the LNP. For example, when the POZ in the cationic-POZ-lipid is PPOZ 500, the cationic POZ-lipid may be included in the LNP in an amount of about 50 to about 80 mol percent of the LNP, or about 60 to about 70 mol percent of the LNP. And, when the POZ in the cationic is PEOZ 700, the cationic POZ-lipid may be included in the LNP in an amount of about 66 to about 80 mol percent of the LNP, or about 65 to about 75 mol percent of the LNP

In one embodiment, the mol percent of cationic POZ-lipid in the LNP is about 5 percent to about 25 percent. In some aspects, the mol percent of cationic POZ-lipid in the LNP is about 8 percent to about 25 percent. In other aspects, the mol percent of cationic POZ-lipid in the LNP is about 10 percent to about 20 percent. In still other aspects, the mol percent of cationic POZ-lipid in the LNP is about 15 percent to about 25 percent. In yet other aspects, the mol percent of cationic POZ-lipid in the LNP is about 10 percent to about 25 percent. In this regard, the remainder of the LNP may be about 50 to about 85 percent sterol lipid, about 0.5 percent to about 10 percent polymer lipid, and about 5 percent to about 15 percent helper lipid. In some embodiments, the mol percent of sterol lipid in the LNP may range from about 55 percent to about 80 percent. In other embodiments, the mol percent of polymer lipid may range from about 0.5 percent to about 10 percent, about 0.5 percent to about 5 percent, or about 1 percent to about 3 percent. In still other embodiments, the mol percent of the helper lipid may range from about 7 percent to about 15 percent, about 8 percent to about 12 percent, or about 8 percent to about 11 percent.

In some embodiments, the LNP may be about 10 percent to about 20 percent cationic POZ-lipid, about 50 percent to about 85 percent sterol lipid, about 0.5 percent to about 10 percent polymer lipid, and about 5 percent to about 15 percent helper lipid. In other embodiments, the LNP may be about 10 percent to about 25 percent cationic POZ-lipid, about 50 percent to about 85 percent sterol lipid, about 0.5 percent to about 10 percent polymer lipid, and about 5 percent to about 15 percent helper lipid. In still other embodiments, the LNP may be about 15 percent to about 25 percent cationic POZ-lipid, about 50 percent to about 85 percent sterol lipid, about 0.5 percent to about 10 percent polymer lipid, and about 5 percent to about 15 percent helper lipid.

In some embodiments, the LNP may be about 40 percent to about 60 percent cationic POZ-lipid, about 10 percent to about 40 percent sterol lipid, about 0.5 percent to about 10 percent polymer lipid, and about 5 percent to about 15 percent helper lipid. In other embodiments, the LNP may be about 60 percent to about 80 percent cationic POZ-lipid, about 10 percent to about 20 percent sterol lipid, about 0.5 percent to about 10 percent polymer lipid, and about 5 percent to about 15 percent helper lipid. In still other embodiments, the LNP may be about 50 percent to about 70 percent cationic POZ-lipid, about 20 percent to about 50 percent sterol lipid, about 0.5 percent to about 10 percent polymer lipid, and about 5 percent to about 15 percent helper lipid.

Similarly, polyplexes may be made in accordance with the present disclosure using the cationic polymers and payloads disclosed herein. In some embodiments, a solution of the cationic polymer may be mixed with a payload solution.

### Administration

The cationic POZ-lipid LNPs of the present disclosure may be delivered to any cell. After in vivo administration of the cationic POZ-lipid LNPs, the payload is released. In this aspect, the cationic POZ-lipid LNPs of the present disclosure may be included a pharmaceutical composition capable of eliciting a treatment for a disorder or disease. For example, pharmaceutical compositions including cationic POZ-lipid LNPs made in accordance with the present disclosure may be used to prevent or treat infectious diseases including, but not limited to, SARS-CoV-2, rabies, influenza, and others by specifically targeting certain immune cells other than the antigen-presenting cells. In addition, pharmaceutical compositions including cationic POZ-lipid LNPs made in accordance with the present disclosure may be used as therapeutics for cancer and genetic diseases. Such pharmaceutical compositions may also include a pharmaceutically acceptable carrier in addition to the cationic POZ-lipid LNPs.

In one embodiment, a pharmaceutical composition including an effective amount of cationic POZ-lipid LNP of the present disclosure can be delivered to an animal. In this aspect, delivery of an effective amount of a cationic POZ-lipid LNP of the present disclosure may occur via subcutaneous, intravenous, intramuscular, intradermal or aerosol routes. In one embodiment, the animal is a human.

### EXAMPLES

The following examples do not limit the invention or the claimed subject-matter. Rather, these examples are intended to further illustrate embodiments of the present disclosure.

### Example 1. Synthesis of PPOZ 1p NMPCA 1p Lipids

### Step 1A. Synthesis of PPOZ-OH 2.6K np NMPCA

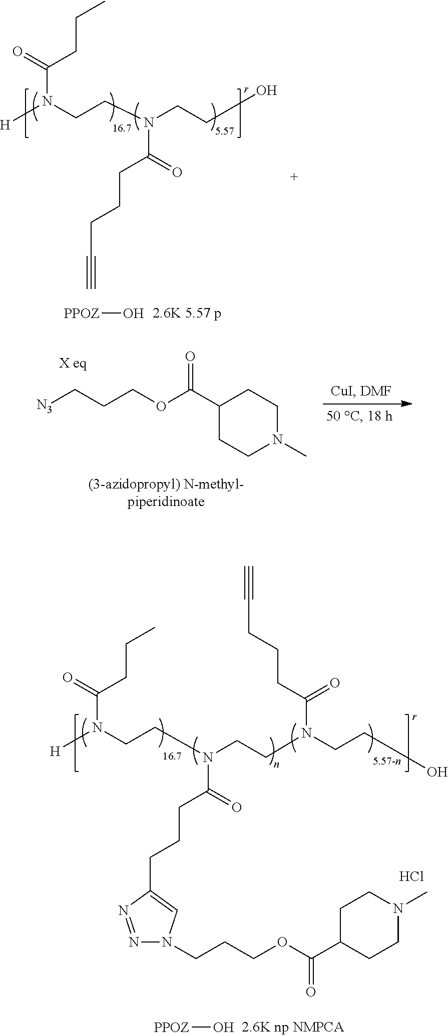

### Step 1B. Synthesis of PPOZ 2.6K 1p NMPCA, (where n=1)

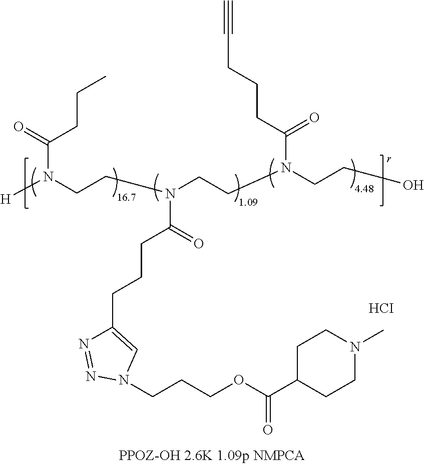

An oven-dried 25-mL round bottomed flask, equipped with a reflux condenser and an argon gas inlet, was charged with PPOZ-OH 2.6K 5.6p (1.00 g, 0.377 mmol, 1.0 eq, Mn 2654 Da) and (3-azidopropyl)-N-methylpiperidinoate (0.102 g, 0.452 mmol, 1.2 eq) followed by a stir bar and THF (10 mL) under argon atmosphere. After addition of CuI (0.0359 g, 0.188 mmol, 0.5 eq), the resulting mixture was stirred for 5 minutes at room temperature and then allowed to stir for 18 hours at 50° C. After cooling down to room temperature, the reaction mixture was quenched by adding 0.5 N aqueous HCl (3 mL) followed by stirring for 5 minutes. The mixture was passed through the Dowex® M4195 column and the column was eluted with a mixture of THF and 2 mN HCl (3:1). The resulting solution was concentrated using a rotary evaporator at 34° C. The residue was purified by silica-gel column chromatography using a Biotage (SNAP Ultra C18 cartridge), with a mixture of 2 mN HCl and methanol as an eluting solvent. After removing MeOH, the resulting aqueous solution was freeze-dried using a lyophilizer to provide the desired product (1.00 g, 91% yield) as a pale-yellow powder.

¹H NMR (Varian, 500 MHz, 10 mg/mL DMSO-d₆, δ) analysis showed the standard backbone signals for PPOZ at 7.91 (br m, 1H, terminal NH), 3.34 (m, 4H, —NCH₂CH₂N— backbone), 3.16 (m, 2H, —N—CH₂), 2.27 and 2.21 (m, total area 2H, —C(═O)CH₂CH₂CH₃), 1.48 (m, 2H, —C(═O)CH₂CH₂CH₃), and 0.84 (m, 3H, —C(═O)CH₂CH₂CH₃). Signals for the pendant group were present at 7.85 (br s, 1H, triazole ring, resulted by ‘click’ reaction), 4.37 (m, 2H, triazole N—CH₂CH₂CH₂—O—), 4.01 (m, 2H, triazole N—CH₂CH₂CH₂—O—), 2.58 (m, total area 6H, piperidyl), 2.15 (br s, 3H, pip-N—CH₃), 2.01 (m, 2H, -piperidyl (CHH)N—CH₃), 1.95 (m, 1H, pip-CH—C(O)O—), and 1.64 (m, 2H, triazole N—CH₂CH₂CH₂—O—). Additionally, two types of pendant pentynyl group peaks appear at 1.77 (m, 2H, backbone-CH₂CH₂CH₂-triazole ring, ‘clicked’) and 1.63 (m, 2H, backbone-CH₂CH₂CH₂C≡CH, intact). The number of pendant groups was determined from the ¹H NMR analysis to be 1.09 by comparing the integrations of pendant group protons and polymer backbone protons, and the calculated molecular weight was 2940 Da.

### Step 2. Synthesis of Azido-Lipids

### Step 2A. Synthesis of 2-[(6-azidohexanoate)methyl]propane-1,3-diyl ditetradecanoate, azido-DT

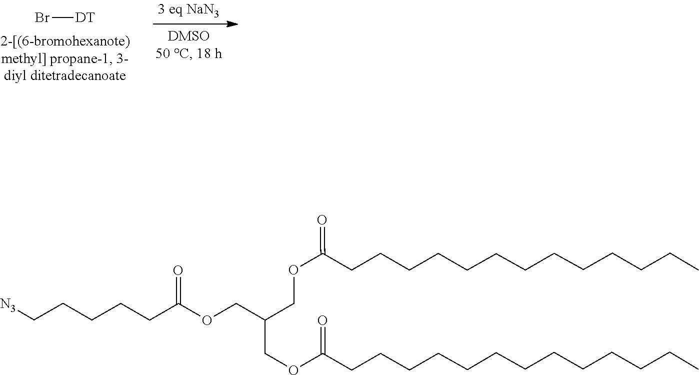

Bromo-DT (30.0 mg, 0.0426 mmol, 1.0 eq), anhydrous DMSO (1 mL), and sodium azide (8.3 mg, 0.128 mmol, 3.0 eq) were added to a 3-dram vial were added, along with a magnetic stirrer bar. The mixture was allowed to stir at 50° C. for 18 hours under an atmosphere of argon. After cooling down to room temperature, the mixture was diluted with water (6 mL) and then extracted with diethyl ether twice (4 mL×2). The combined organic phases were washed with water (2 mL), then brine, dried over Na₂SO₄, filtered, and concentrated to give 28.2 mg (99.4% yield) of desired product as a colorless clear oil.

¹H NMR (Varian, 500 MHz, 10 mg/mL DMSO-d₆) δ 0.84 (t, 6H, J=6.7 Hz, —(CH₂)₁₀CH₃), 1.22 (m, 40H, —(CH₂)₁₀CH₃), 1.59 (m, 6H, —OC(═O)CH₂CH₂— and 2H, N₃CH₂CH₂CH₂—), 1.69 (m, 2H, N₃CH₂CH₂—), 2.27 (m, 1H, CH[(CH₂)O-]₃ and 6H, —OC(═O)CH₂CH₂—), 3.30 (t, 2H, J=6.0 Hz, N₃CH₂—), 4.05 (d, 6H, J=6.0 Hz, CH[(CH₂)O-]₃).

### Step 2B. Synthesis of 1,1′-[2-[(6-azidohexanonate)methyl]-1,3-propanediyl]dipentadecan-7-yl adipate, azido-DPA

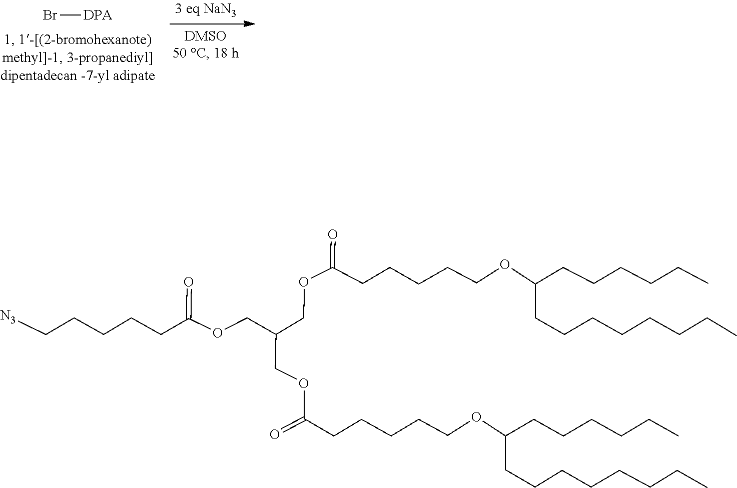

Bromo-DPA (100.0 mg, 0.104 mmol, 1.0 eq), anhydrous DMSO (1 mL), and sodium azide (20.3 mg, 0.312 mmol, 3.0 eq) were added to a 3-dram vial, along with a magnetic stirrer bar. The mixture was allowed to stir at 50° C. for 18 hours under an atmosphere of argon. After cooling down to room temperature, the mixture was diluted with water (6 mL) and then extracted with diethyl ether twice (4 mL×2). The combined organic phases were washed with water (2 mL), then brine, dried over Na₂SO₄, filtered, and concentrated to give 92.5 mg (96.4% yield) of desired product as a colorless clear oil.

¹H NMR (Varian, 500 MHz, 10 mg/mL DMSO-d₆) δ 0.83 (t, 12H, J=6.0 Hz, —CH₂CH₃), 1.21 (m, 16H, —(CH₂)₄CH₃ and 24H, —(CH₂)₆CH₃), 1.30 (m, 2H, N₃CH₂CH₂—), 1.45 (m, 8H, —OCH(CH₂CH₂—)₂), 1.51 (m, 10H, —OC(═O)CH₂CH₂— and 2H, N₃CH₂CH₂CH₂—), 2.25 (m, 1H, CH[(CH₂)O-]₃), 2.29 (10H, —OC(═O)CH₂CH₂—), 3.30 (t, 2H, J=10.0 Hz, N₃CH₂—), 4.05 (d, 6H, J=5.5 Hz, CH[(CH₂)O-]₃), 4.76 (m, 2H, —O—CH[(CH₂)—]₂).

### Step 2C. Synthesis of 2-(azidomethyl)-1,2,3,-(9Z)-trioctadeca-9-enoate, azido-TOE

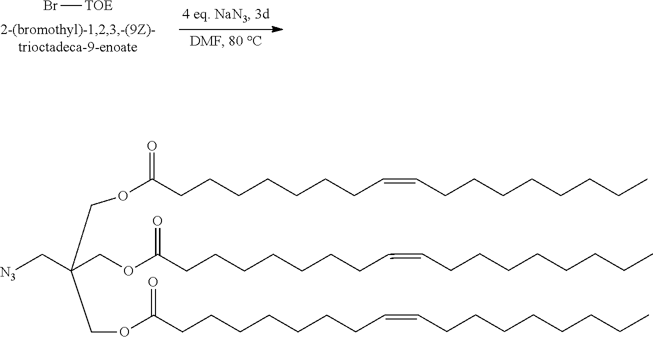

Bromo-TOE (300.0 mg, 0.296 mmol, 1.0 eq), anhydrous DMF (5 mL), and sodium azide (77.0 mg, 1.185 mmol, 4.0 eq) were added to a 6-dram vial, along with a magnetic stirrer bar. The mixture was allowed to stir at 80° C. for 3 days under an atmosphere of argon. After cooling down to room temperature, the mixture was diluted with water (30 mL) and then extracted with diethyl ether twice (20 mL×2). The combined organic phases were washed with water (10 mL), then brine, dried over Na₂SO₄, filtered, and concentrated to give 271.3 mg (95.9% yield) of desired product as a colorless clear oil.

¹H NMR (Varian, 500 MHz, 10 mg/mL DMSO-d₆) δ 0.88 (t, 9H, J=6.0 Hz, —(CH₂)₇CH₃), 1.29 (m, 66H, —(CH₂)₅ CH₂CH═CHCH₂(CH₂)₆CH₃), 1.61 (m, 6H, —OC(═O)CH₂CH₂—), 2.01 (m, 12H, —CH₂CH═CHCH₂—), 2.32 (t, 6H, J=8.0 Hz, —OC(═O)CH₂—), 3.45 (s, 2H, N₃CH₂—), 4.07 (s, 6H, —C[(CH₂)O-]₃), 5.35 (m, 6H, —CH₂CH═CHCH₂—).

### Step 3. Synthesis of PPOZ-OH 2.6K 1p NMPCA-Lipids from PPOZ-OH 2.6K 1p NMPCA

Using the PPOZ-OH 2.6K 1p NMPCA in Step 1B and azido-Lipids from Step 2, cationic-POZ-lipids were formed with a click reaction as shown below.

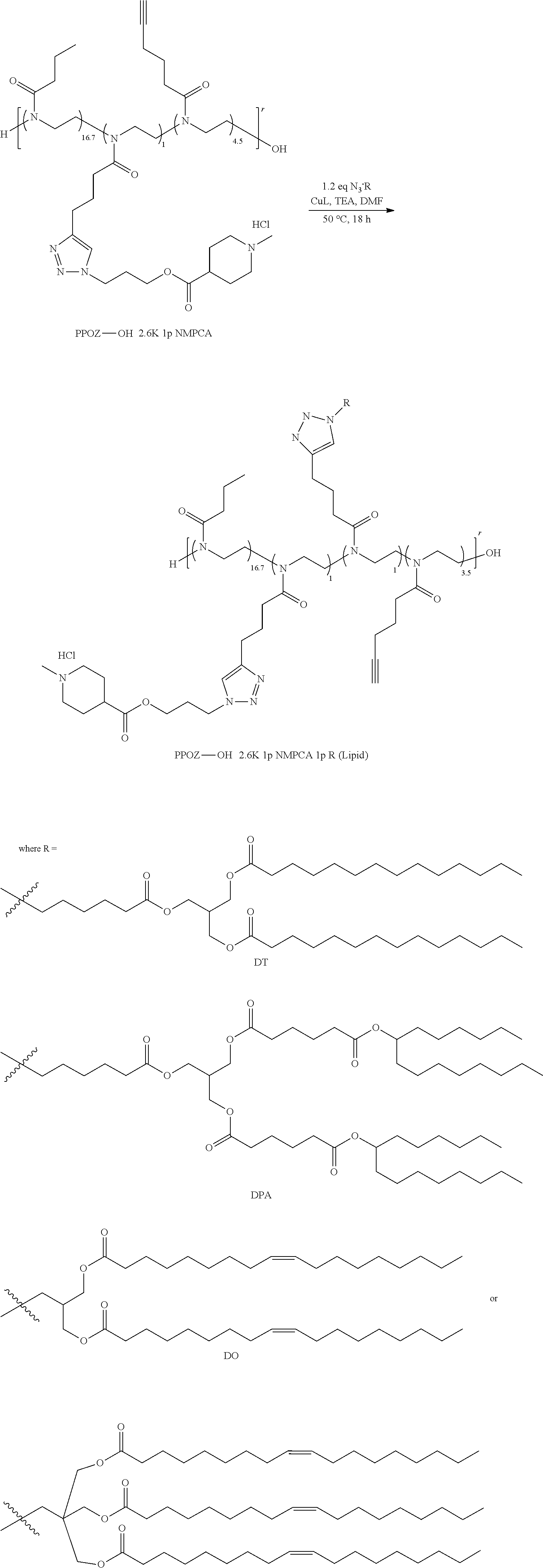

### Step 3A. Synthesis of PPOZ 2.6K 1p NMPCA 1p DT

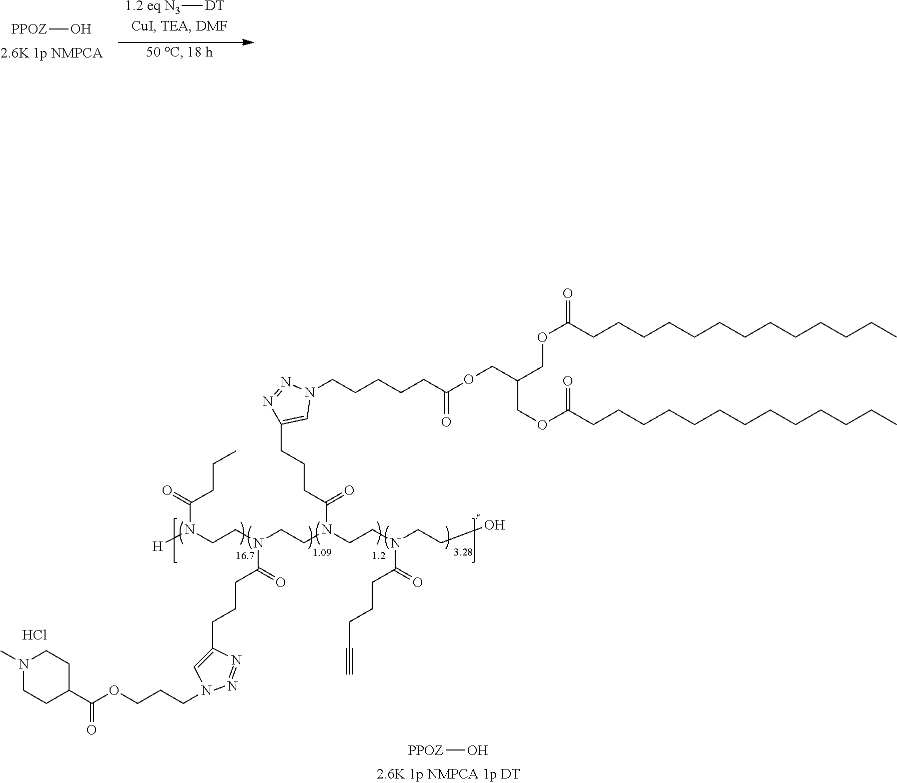

An oven-dried 3-dram vial, equipped with a reflux condenser and an argon gas inlet, was charged with PPOZ-OH 2.6K 1p NMPCA (50.0 mg, 0.017 mmol, 1.0 eq, Mn 2940 Da) and 2-[(6-azidohexanoate)methyl]propane-1,3-diyl ditetradecanoate (azido-DT, 13.6 mg, 0.0204 mmol, 1.2 eq) followed by a stir bar and DMF (1.5 mL) under an argon atmosphere. After addition of CuI (1.3 mg, 0.0068 mmol, 0.4 eq) and TEA (7.1 μL, 0.051 mmol, 3 eq), the resulting mixture was stirred for 5 minutes at room temperature and then allowed to stir for 18 hours at 50° C. After cooling down to room temperature, the reaction mixture was quenched by adding 0.5 N aqueous HCl (0.4 ff mL) followed by stirring for 5 minutes. The mixture was passed through the Dowex® M4195 column and the column was eluted with a mixture of DMF and 2 mN HCl (3:1). The resulting solution was concentrated using a rotary evaporator at 34° C. The residue was purified by silica-gel column chromatography using a Biotage (Sfar C18 cartridge), with a mixture of water and methanol as an eluting solvent. After removing MeOH, the resulting aqueous solution was freeze-dried using a lyophilizer to provide the desired product (11.5 mg, 18.8% yield) as an off-white powder.

¹H NMR (Varian, 500 MHz, 10 mg/mL DMSO-d₆, δ) analysis showed the standard backbone signals for PPOZ at 0.83 (m, 3H, —C(═O)CH₂CH₂CH₃), 1.48 (m, 2H, —C(═O)CH₂CH₂CH₃), 2.21 and 2.27 (m, total area 2H, —C(═O)CH₂CH₂CH₃), 3.16 (m, 2H, —N—CH₂), 3.34 (m, 4H, —NCH₂CH₂N— backbone), and 7.91 (br m, 1H, terminal NH). Signals for the pendant N-methyl piperidinyl group were present at δ 1.64 (m, 2H, triazole N—CH₂CH₂CH₂—O—), 1.95 (m, 1H, pip-CH—C(O)O—), 2.01 (m, 2H, -piperidyl (CHH)N—CH₃), 2.15 (br s, 3H, pip-N—CH₃), 2.58 (m, total area 6H, piperidyl), 4.01 (m, 2H, triazole N—CH₂CH₂CH₂—O—), 4.38 (m, 2H, triazole N—CH₂CH₂CH₂—O—), and 7.85 (br s, 1H, triazole ring). The attachment of azido lipid (N₃-DPA) was proved by the proton signals at δ 0.83 (t, 6H, J=6.7 Hz, —(CH₂)₁₀CH₃), 1.21 (m, 40H, —(CH₂)₁₀CH₃), 4.04 (m, 6H, CH[(CH₂)O-]₃), 4.25 (m, 2H, triazole N—CH₂—), 7.83 (br s, 1H, triazole ring, resulted by ‘Click’ reaction). Additionally, two types of pendant pentynyl group peaks appear at 1.77 (m, 2H, backbone-CH₂CH₂CH₂-triazole ring, ‘clicked’) and 1.63 (m, 2H, backbone-CH₂CH₂CH₂C≡CH, intact). The number of pendant groups was determined from the ¹H NMR analysis to be 1.09 for the NMPCA moiety and 1.0 for the DT lipid moiety by comparing the integrations of pendant group protons and polymer backbone protons, and the calculated molecular weight was 3600 Da.

### Step 3B. Synthesis of PPOZ 2.6K 1p NMPCA 1p DPA

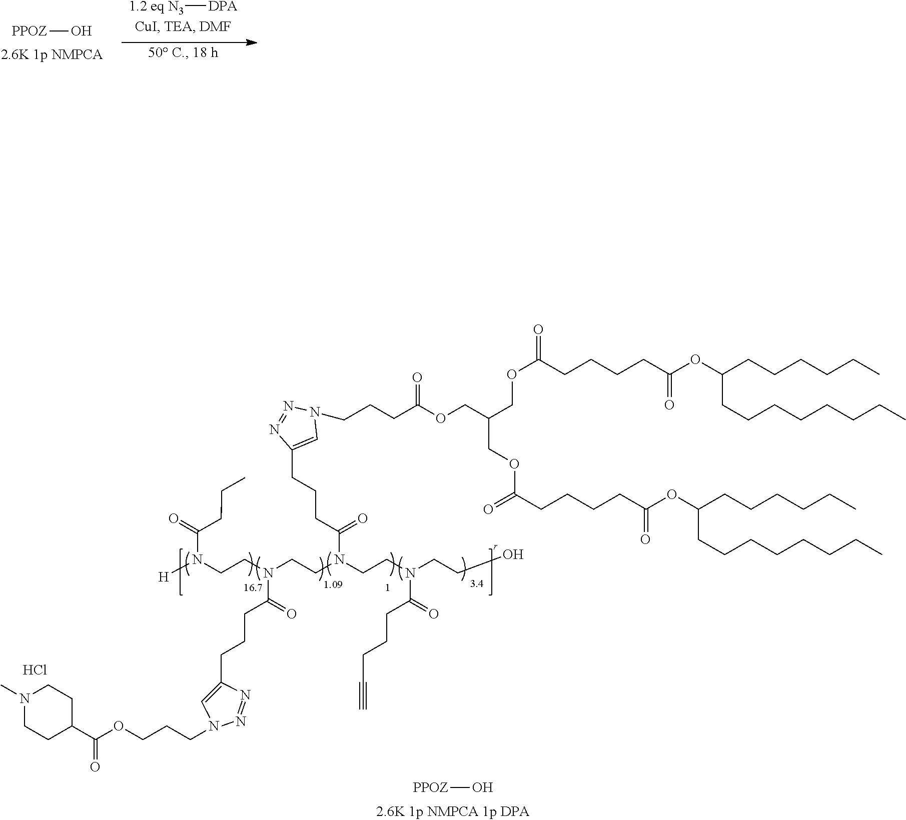

An oven-dried 3-dram vial, equipped with a reflux condenser and an argon gas inlet, was charged with PPOZ-OH 2.6K 1p NMPCA (50.0 mg, 0.017 mmol, 1.0 eq, Mn 2940 Da) and 1,1′-[2-[(6-azidohexanonate)methyl]-1,3-propanediyl]dipentadecan-7-yl adipate (azido-DPA, 21.6 mg, 0.0204 mmol, 1.2 eq) followed by a stir bar and DMF (1.5 mL) under an argon atmosphere. After addition of CuI (1.3 mg, 0.0068 mmol, 0.4 eq) and TEA (7.1 μL, 0.051 mmol, 3 eq), the resulting mixture was stirred for 5 minutes at room temperature and then allowed to stir for 18 hours at 50° C. After cooling down to room temperature, the reaction mixture was quenched by adding 0.5 N aqueous HCl (0.4 ff mL) followed by stirring for 5 minutes. The mixture was passed through the Dowex® M4195 column and the column was eluted with a mixture of DMF and 2 mN HCl (3:1). The resulting solution was concentrated using a rotary evaporator at 34° C. The residue was purified by silica-gel column chromatography using a Biotage (Sfar C18 cartridge), with a mixture of water and methanol as an eluting solvent. After removing MeOH, the resulting aqueous solution was freeze-dried using a lyophilizer to provide the desired product (6.9 mg, 10.5% yield) as an off-white powder.

¹H NMR (Varian, 500 MHz, 10 mg/mL DMSO-d₆, δ) analysis showed the standard backbone signals for PPOZ at 0.83 (m, 3H, —C(═O)CH₂CH₂CH₃), 1.48 (m, 2H, —C(═O)CH₂CH₂CH₃), 2.21 and 2.27 (m, total area 2H, —C(═O)CH₂CH₂CH₃), 3.16 (m, 2H, —N—CH₂), 3.34 (m, 4H, —NCH₂CH₂N— backbone), and 7.91 (br m, 1H, terminal NH). Signals for the pendant N-methyl piperidinyl group were present at δ 1.64 (m, 2H, triazole N—CH₂CH₂CH₂—O—), 1.95 (m, 1H, pip-CH—C(O)O—), 2.01 (m, 2H, -piperidyl (CHH)N—CH₃), 2.15 (br s, 3H, pip-N—CH₃), 2.58 (m, total area 6H, piperidyl), 4.01 (m, 2H, triazole N—CH₂CH₂CH₂—O—), 4.38 (m, 2H, triazole N—CH₂CH₂CH₂—O—), and 7.85 (br s, 1H, triazole ring). The attachment of azido lipid (N₃-DPA) was proved by the proton signals at δ 0.83 (m, 12H, —CH₂CH₃), 1.22 (m, 16H, —(CH₂)₄CH₃ and 24H, —(CH₂)₆CH₃), 1.45 (m, 8H, —OCH(CH₂CH₂—)₂), 1.51 (m, 10H, —OC(═O)CH₂CH₂— and 2H, N₃CH₂CH₂CH₂—), 2.25 (m, 1H, CH[(CH₂)O-]₃), 2.29 (10H, —OC(═O)CH₂CH₂—), 4.04 (d, 6H, J=5.5 Hz, CH[(CH₂)O-]₃), 4.25 (m, 2H, triazole N—CH₂—), 4.75 (m, 2H, —O—CH[(CH₂)—]₂), 7.86 (br s, 1H, triazole ring, resulted by ‘Click’ reaction). Additionally, two types of pendant pentynyl group peaks appear at 1.77 (m, 2H, backbone-CH₂CH₂CH₂-triazole ring, ‘clicked’) and 1.63 (m, 2H, backbone-CH₂CH₂CH₂C≡CH, intact). The number of pendant groups was determined from the ¹H NMR analysis to be 1.09 for the NMPCA moiety and 1.0 for the DPA lipid moiety by comparing the integrations of pendant group protons and polymer backbone protons, and the calculated molecular weight was 3860 Da.

### Step 3C. Synthesis of PPOZ 2.6K 1p NMPCA 1p DO

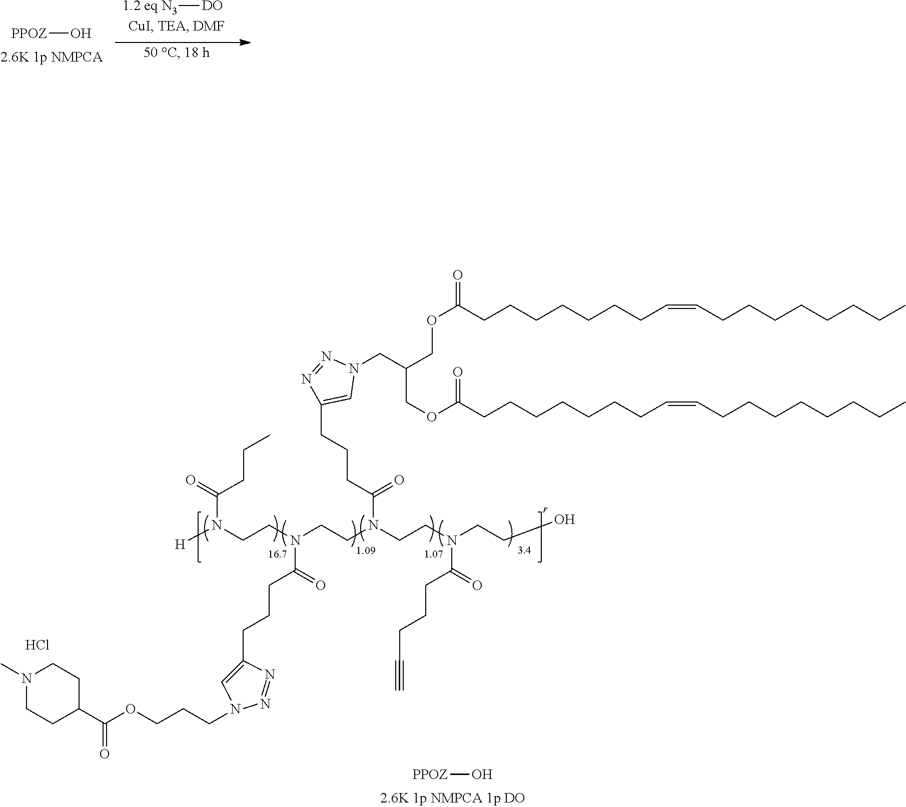

An oven-dried 25-mL round bottomed flask, equipped with a reflux condenser and an argon gas inlet, was charged with PPOZ-OH 2.6K 1p NMPCA (300.0 mg, 0.102 mmol, 1.0 eq, Mn 2940 Da) and 2-(azidomethyl)propane-1,3-diyl dioleate (azido-DO, 80.8 mg, 0.122 mmol, 1.2 eq) followed by a stir bar and DMF (6 mL) under an argon atmosphere. After addition of CuI (8.0 mg, 0.052 mmol, 0.5 eq) and TEA (42.7 μL, 0.306 mmol, 3 eq), the resulting mixture was stirred for 5 minutes at room temperature and then allowed to stir for 18 hours at 50° C. After cooling down to room temperature, the reaction mixture was quenched by adding 0.5 N aqueous HCl (2 mL) followed by stirring for 5 minutes. The mixture was passed through the Dowex® M4195 column and the column was eluted with a mixture of DMF and 2 mN HCl (3:1). The resulting solution was concentrated using a rotary evaporator at 34° C. The residue was purified by silica-gel column chromatography using a Biotage (Sfar C18 cartridge), with a mixture of water and methanol as an eluting solvent. After removing MeOH, the resulting aqueous solution was freeze-dried using a lyophilizer to provide the desired product (82.4 mg, 22.5% yield) as an off-white powder.

¹H NMR (Varian, 500 MHz, 10 mg/mL DMSO-d₆, δ) analysis showed the standard backbone signals for PPOZ at 0.83 (m, 3H, —C(═O)CH₂CH₂CH₃), 1.48 (m, 2H, —C(═O)CH₂CH₂CH₃), 2.21 and 2.27 (m, total area 2H, —C(═O)CH₂CH₂CH₃), 3.16 (m, 2H, —N—CH₂), 3.34 (m, 4H, —NCH₂CH₂N— backbone), and 7.91 (br m, 1H, terminal NH). Signals for the pendant N-methyl piperidinyl group were present at δ 1.64 (m, 2H, triazole N—CH₂CH₂CH₂—O—), 1.95 (m, 1H, pip-CH—C(O)O—), 2.01 (m, 2H, -piperidyl (CHH)N—CH₃), 2.15 (br s, 3H, pip-N—CH₃), 2.58 (m, total area 6H, piperidyl), 4.01 (m, 2H, triazole N—CH₂CH₂CH₂—O—), 4.38 (m, 2H, triazole N—CH₂CH₂CH₂—O—), and 7.85 (br s, 1H, triazole ring). The attachment of azido lipid (N₃-DO) was proved by the proton signals at δ 0.83 (m, 6H, —(CH₂)₆CH₃), 1.22 (m, 40H, —(CH₂)₄CH₂ CH═CHCH₂(CH₂)₆CH₃), 1.64 (m, 4H, —OC(═O)CH₂CH₂—), 1.96 (m, 8H, —CH₂CH═CHCH₂—), 2.26 (m, 1H, CH[(CH₂)₃, 2.35 (m, 4H, —OC(═O)CH₂—), 4.01 (m, 4H, —CH[(CH₂)O-]₂), 4.38 (m, 2H, triazole N—CH₂—), 5.30 (m, 4H, —CH₂CH═CHCH₂—), 7.84 (br s, 1H, triazole ring, resulted by ‘Click’ reaction). Additionally, two types of pendant pentynyl group peaks appear at 1.77 (m, 2H, backbone-CH₂CH₂CH₂-triazole ring, ‘clicked’) and 1.63 (m, 2H, backbone-CH₂CH₂CH₂C≡CH, intact). The number of pendant groups was determined from the ¹H NMR analysis to be 1.09 for the NMPCA moiety and 1.07 for the DO lipid moiety by comparing the integrations of pendant group protons and polymer backbone protons, and the calculated molecular weight was 3600 Da.

### Step 3D. Synthesis of PPOZ 2.6K 1p NMPCA 1p TOE

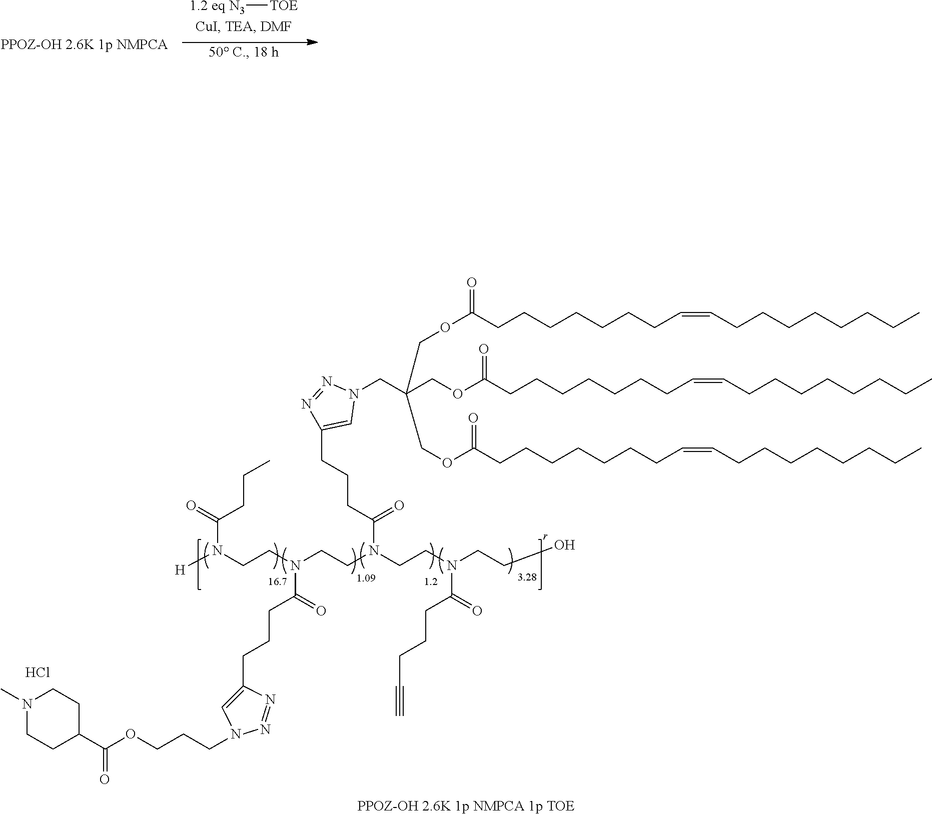

An oven-dried 25-mL round bottomed flask, equipped with a reflux condenser and an argon gas inlet, was charged with PPOZ-OH 2.6K 1p NMPCA (275.3 mg, 0.0936 mmol, 1.0 eq, Mn 2940 Da) and 2-(azidomethyl)-1,2,3,-(9Z)-trioctadeca-9-enoate (azido-TOE, 89.4 mg, 0.0936 mmol, 1.2 eq) followed by a stir bar and DMF (8 mL) under an argon atmosphere. After addition of CuI (7.1 mg, 0.037 mmol, 0.4 eq) and TEA (39.1 μL, 0.281 mmol, 3 eq), the resulting mixture was stirred for 5 minutes at room temperature and then allowed to stir for 18 hours at 50° C. After cooling down to room temperature, the reaction mixture was quenched by adding 0.5 N aqueous HCl (2 mL) followed by stirring for 5 minutes. The mixture was passed through the Dowex® M4195 column and the column was eluted with a mixture of DMF and 2 mN HCl (3:1). The resulting solution was concentrated using a rotary evaporator at 34° C. The residue was purified by silica-gel column chromatography using a Biotage (Sfar C18 cartridge), with a mixture of water and methanol as an eluting solvent. After removing MeOH, the resulting aqueous solution was freeze-dried using a lyophilizer to provide the desired product (30.1 mg, 8.3% yield) as an off-white powder.

¹H NMR (Varian, 500 MHz, 10 mg/mL DMSO-d₆, δ) analysis showed the standard backbone signals for PPOZ at 0.83 (m, 3H, —C(═O)CH₂CH₂CH₃), 1.48 (m, 2H, —C(═O)CH₂CH₂CH₃), 2.21 and 2.27 (m, total area 2H, —C(═O)CH₂CH₂CH₃), 3.16 (m, 2H, —N—CH₂), 3.34 (m, 4H, —NCH₂CH₂N— backbone), and 7.91 (br m, 1H, terminal NH). Signals for the pendant N-methyl piperidinyl group were present at δ 1.64 (m, 2H, triazole N—CH₂CH₂CH₂—O—), 1.95 (m, 1H, pip-CH—C(O)O—), 2.01 (m, 2H, -piperidyl (CHH)N—CH₃), 2.15 (br s, 3H, pip-N—CH₃), 2.58 (m, total area 6H, piperidyl), 4.01 (m, 2H, triazole N—CH₂CH₂CH₂—O—), 4.38 (m, 2H, triazole N—CH₂CH₂CH₂—O—), and 7.85 (br s, 1H, triazole ring). The attachment of azido lipid (N₃-DO) was proved by the proton signals at δ 0.88 (t, 9H, J=6.0 Hz, —(CH₂)₇CH₃), 1.21 (m, 66H, —(CH₂)₅ CH₂CH═CHCH₂(CH₂)₆CH₃), 1.64 (m, 6H, —OC(═O)CH₂CH₂—), 1.95 (m, 12H, —CH₂CH═CHCH₂—), 3.99 (s, 6H, —C[(CH₂)O-]₃), 4.46 (s, 2H, triazole N—CH₂—), 5.29 (m, 6H, —CH₂CH═CHCH₂—), 7.78 (m, 1H, triazole ring, resulted by ‘Click’ reaction). Additionally, two types of pendant pentynyl group peaks appear at 1.77 (m, 2H, backbone-CH₂CH₂CH₂-triazole ring, ‘clicked’) and 1.63 (m, 2H, backbone-CH₂CH₂CH₂C≡CH, intact). The number of pendant groups was determined from the ¹H NMR analysis to be 1.09 for the NMPCA moiety and 1.0 for the TOE lipid moiety by comparing the integrations of pendant group protons and polymer backbone protons, and the calculated molecular weight was 3895 Da.

### Step 4. Preparation of Lipid Nanoparticles Containing saRNA and PPOZ-OH 2.6K NMPCA-Lipid

Using the PPOZ-OH 2.6K 1p NMPCA 1p Lipids in Step 3, LNPs were formed as described below.

The self-amplifying RNA (saRNA) was the Enhanced Green Fluorescent Protein encoded with the EGFP gene from the jellyfish, Aequorea victoria (Creative Biogene, PMSAR-0001). The single-stranded RNA has 8,528 nucleotides and the EGFP has a molecular weight of 27 kDa. The supplied solution of 1.687 μg/μL in water, was diluted with 20 mM sodium acetate buffer, pH 4.5, to prepare a solution of 0.01 μg/μL concentration.

Each lipid stock solution of PPOZ-OH 2.6K 1p NMPCA-Lipids (from Steps 3A-3D), 1,2-distearoyl-sn-glycero-3-phosphocholine (DSPC, Bachem 4005619), cholesterol (VWR 0433) and DMA-PEOZ 2k (as described in U.S. Pat. No. 12,233,132, the entire disclosure of which is incorporated by reference herein) were prepared in ethanol (EMD Millipore EX0276-4). The stock solutions were mixed so that the concentration of lipid mixture was prepared as 2 mg/mL, with varying mol % of cholesterol and N/P ratios as shown in Table 1 below.

TABLE 1
saRNA-LNP systems containing PPOZ-OH 1p NMPCA-Lipids¹

| | PPOZ-OH 1p NMPCA-lipid/DSPC/ | |
|---|---|---|
| LNP # | Cholesterol/DMA-PEOZ 2k mol % | N/P ratio |
| 1 | 10:10:78.4:1.6 | 2.7 |
| 2 | 20:10:68.4:1.6 | 3.8 |
| 3 | 30:10:58.4:1.6 | 4.5 |
| 4 | 40:10:48.4:1.6 | 4.9 |
| 5 | 50:10:38.4:1.6 | 5.1 |

¹with 10 to 50 mol % at the expense of cholesterol from 78.4 to 38.4 mol % (keeping DSPC and DMA-PEOZ 2k mol % constant)

The PPOZ 2.6K 1p NMPCA-Lipid in ethanol was mixed with saRNA in 20 mM Na Acetate, pH 4.5 at a ratio of three parts aqueous to one part organic using a pipette via hand mixing and then diluted 10 times with 25 mM Tris-HCl, pH 7.4. The resulting mixture was incubated for one hour at room temperature to reduce ethanol content in the lipid nanoparticles.

### Example 2: Characterization of saRNA-LNPs Containing PPOZ-OH 2.6K NMPCA-Lipids

Each lipid stock solution of PPOZ-OH 2.6K 1p NMPCA-Lipid (from Step 3 of Example 1), 1,2-distearoyl-sn-glycero-3-phosphocholine (DSPC, Bachem 4005619), cholesterol (VWR 0433) and PEOZ 2K DMA (as described in U.S. Pat. No. 12,233,132) were prepared in ethanol (EMD Millipore EX0276-4). The stock solutions were mixed so that the concentration of lipid mixture was prepared as 2 mg/mL, with varying mol % of cholesterol.

Selected molar ratios for four lipid mixture were as shown in Table 2 below. The PPOZ 2.6K 1p NMPCA-Lipid in ethanol was mixed with saRNA in 20 mM Na Acetate, pH 4.5 at a ratio of three parts aqueous to one part organic using a pipette via hand mixing and then diluted 10 times with 25 mM Tris-HCl, pH 7.4. The resulting mixture was incubated for one hour at room temperature to reduce ethanol content in the lipid nanoparticles.

TABLE 2
Molar Ratios

| | PPOZ-OH 1p NMPCA-lipid/DSPC/ |
|---|---|
| PPOZ-OH 1p NMPCA-lipid | Cholesterol/DMA-PEOZ 2K mol % |
| PPOZ-OH 2.6k 1p NMPCA-DO | 25:10:63.4:1.6 |
| PPOZ-OH 2.6k 1p NMPCA-DPA | 25:10:63.4:1.6 |
| PPOZ-OH 2.6k 1p NMPCA-TOE | 30:10:58.4:1.6 |
| PPOZ-OH 2.6k 1p NMPCA-DT | 40:10:48.4:1.6 |

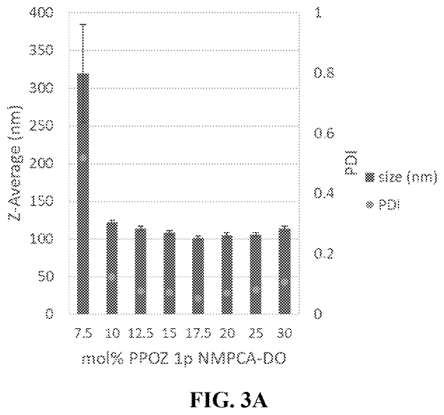

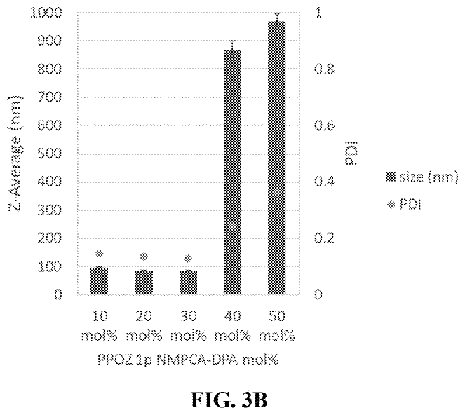

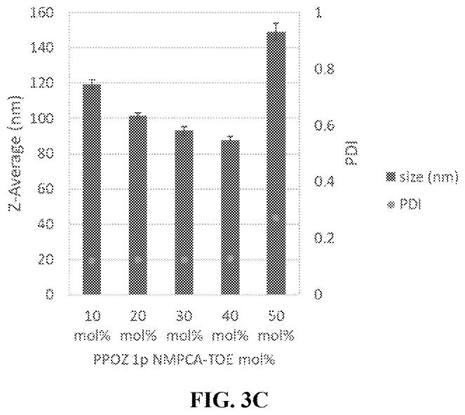

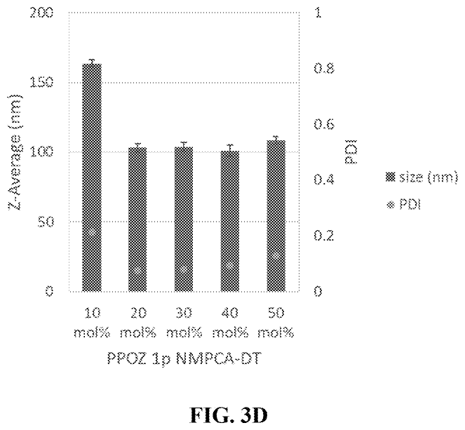

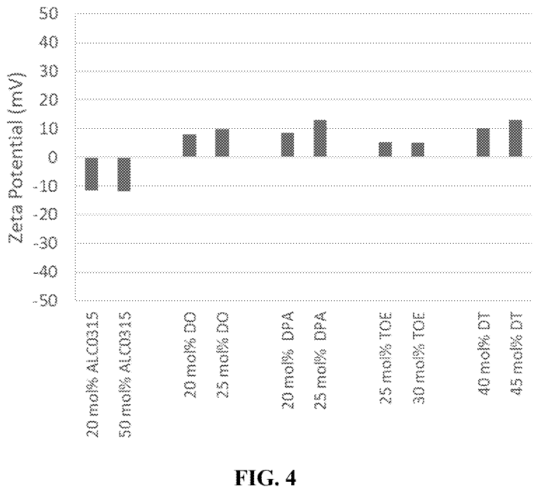

The size of the formulated saRNA-LNPs was measured using dynamic light scattering (DLS) (Zetasizer Ultra, Malvern Panalytical). FIGS. 3A-3D shows the particle size of the LNPs averaged after three measurements ranged between 90 to 150 nm with low PDI (<0.2) at a range of 10-30 mol % PPOZ 1p NMPCA-Lipid. The zeta potential of the saRNA-LNPs was measured using a Zetasizer Ultra (Malvern Panalytical) with electrophoretic light scattering capabilities. The saRNA-LNPs were diluted eight times with 25 mM Tris-HCl, pH 7.5, buffer to measure the zeta potential. FIG. 4 shows the zeta potential recorded after measurements ranged from 5 to 10 mV.

The saRNA encapsulation efficiency was determined by the Quant-iT RiboGreen RNA assay (Life Technologies). saRNA-LNPs were first diluted with 1×TE buffer, pH 8.0, in the absence or presence of 2% Triton-X 100 in a black 96-well plate. To set up a standard curve, 10 μg/mL saRNA was serially diluted with 1×TE buffer, pH 8.0, in the absence or presence of 2% Triton-X 100. Then the plate was transferred to the 37° C. for 10 min incubation followed by adding 100 μL of Ribogreen dye in 1×TE buffer to each well to bind saRNA released from LNPs. Fluorescence was measured by a Varioskan LUX (Invitrogen) at 485 nm excitation and 528 nm emission. saRNA encapsulation efficiency (EE) was calculated by subtracting unencapsulated saRNA from total saRNA.

The apparent pKa of the saRNA-LNPs was determined using 2-(p-toluidino)-6-naphthalenesulfonic acid (TNS) binding assay. TNS was prepared as a 0.2 mM stock solution in water. LNPs were diluted to have a lipid concentration of approximately 4 μM in 0.1 mL of buffers with the pH from 3 to 9, containing 5 μM TNS. Fluorescence was measured by a Varioskan LUX (Invitrogen) at 320 nm excitation and 450 nm emission. A sigmoidal best fit analysis was performed to the fluorescence data by non-linear regression analysis using GraphPad Prism 10 (GraphPad Software). The pKa values in Table 3 were measured as the pH resulted in half-maximal fluorescence intensity normalized to the lowest fluorescence measurement.

TABLE 3
Characterization of saRNA-LNPs with ALC0315
and PPOZ-OH 2.6k 1p NMPCA-Lipid

| | Size | | Zeta potential | EE | Apparent |
|---|---|---|---|---|---|
| | (nm) | PDI | (mV) | (%) | pKa |
| ALC0315 | 89.8 | 0.077 | −11.9 | 94.8 | 6.28 |
| PPOZ-OH 2.6k 1p | 102.2 | 0.058 | 9.9 | 92.7 | 6.74 |
| NMPCA-DO | | | | | |
| PPOZ-OH 2.6k 1p | 81.9 | 0.151 | 13.0 | 86.4 | 6.29 |
| NMPCA-DPA | | | | | |
| PPOZ-OH 2.6k 1p | 106.0 | 0.149 | 5.2 | 93.0 | 6.41 |
| NMPCA-TOE | | | | | |
| PPOZ-OH 2.6k 1p | 103.9 | 0.105 | 10.2 | 97.7 | 6.97 |
| NMPCA-DT | | | | | |

### Example 3: Determination of LNP Payload Delivery and Gene Targeting in HEK293-loxP-GFP-RFP Cells by In-Vitro Transfection with a Cre Recombinase mRNA

The messenger RNA (mRNA) was a capped and polyadenylated messenger RNA encoding the Cre recombinase fused to a nuclear localization sequence (NLS) gene from the jellyfish, Aequorea victoria (TriLink Biotechnologies, L-8111). Cre recombinase is a tyrosine recombinase that catalyzes recombination between two loxP sites. The single-stranded RNA has 1,351 nucleotides and the NLS-Cre recombinase has a molecular weight of 40.2 kDa. The supplied solution of 1.0 μg/μL in water, was diluted with 20 mM sodium acetate buffer, pH 4.5, to prepare a solution of 0.01 μg/μL concentration.

LNP lipid stocks were prepared in ethanol at selected molar ratios of PPOZ 2.6K-OH 2.6k NMPCA-Lipids (Step 3 of Example 1), 1,2-distearoyl-sn-glycero-3-phosphocholine (DSPC, Bachem 4005619), cholesterol (VWR 0433) and DMA-PEOZ 2k (as described in U.S. Pat. No. 12,233,132) in EtOH at a lipid stock concentration of 2 mg/mL. The saRNA aqueous stock was hand-mixed with the lipid stocks at a 3:1 ratio and diluting to a 0.75 ng/μL saRNA concentration with 25 mM Tris, pH 7.5. The resulting mixture was incubated for one hour at room temperature to reduce ethanol content in the lipid nanoparticles.

Human Embryonic Kidney (HEK) 293-loxP-GFP-RFP (Puro), color-Switch CRE reporter cells (GenTarget Inc) are a HEK293 cell line with an integrated LoxP target in the host cell genome. The cell line incorporates LoxP sites and a GFP gene downstream of the promotor with a stop codon following. Once the CRE protein is present, the gene fragment between the two LoxP sites is deleted and as a result, the RFP is then expressed.

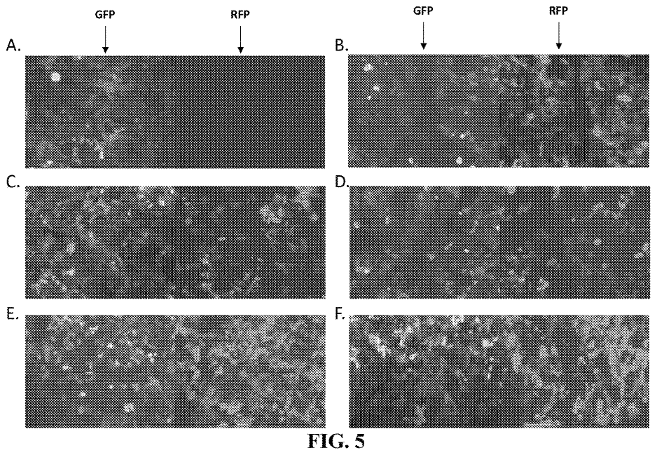

For demonstration of gene targeting with the LNP delivery constructs of the present disclosure, the HEK293-loxP cells were seeded in a 96 well cell treated plate (Corning) at a density of 25,000 cells/well in 200 μL of DMEM media (Corning) with 10% FBS (Corning). The HEK293-loxP plate was incubated at 37° C./5% CO₂ overnight to a confluency of about 60 to 70 percent. Media was replaced in half the wells with Opti-MEM (Gibco) media prior to transfection. The remaining wells were transfected in the cDMEM media. Dose amounts of the LNP formulations were calculated based on CleanCap M6 Cre mRNA (TriLink Biotechnologies) concentration and 50 ng of each dose was added to the plate wells. The plate was returned to the incubator to allow for cellular uptake of the formulation mRNA. After four hours, the wells containing Opti-MEM were replaced with cDMEM media and the plate was returned to incubator for 48 hours. Cells were analyzed on an EVOS-M7000 microscope (Invitrogen) for cellular vitality and expression of Green Fluorescent Protein (GFP) and Red Fluorescent Protein (RFP) signals were visualized using GFP and Texas Red filters. As shown in FIG. 5, the color switch from GFP to RFP expression corresponds to positive gene targeting of the LoxP-flanked target by CRE recombinase.

### Example 4: Cell Viability Assay for Lipid Nanoparticles Containing saRNA and PPOZ-OH 2.6K NMPCA-Lipid

LNP lipid stocks were prepared in ethanol at selected molar ratios of PPOZ 2.6K-OH 2.6k NMPCA-Lipids (Step 3 of Example 1), 1,2-distearoyl-sn-glycero-3-phosphocholine (DSPC, Bachem 4005619), cholesterol (VWR 0433) and DMA-PEOZ 2k (as described in U.S. Pat. No. 12,233,132) in EtOH at a lipid stock concentration of 2 mg/mL. The saRNA aqueous stock was hand-mixed with the lipid stocks at a 3:1 ratio and diluting to a 0.75 ng/μL saRNA concentration with 25 mM Tris, pH 7.5. The resulting mixture was incubated for one hour at room temperature to reduce ethanol content in the lipid nanoparticles.

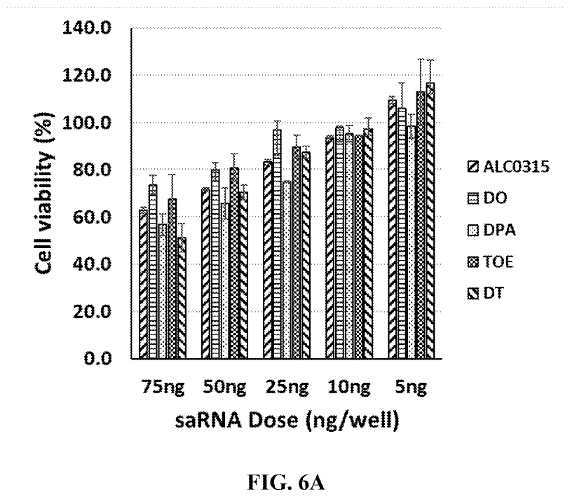

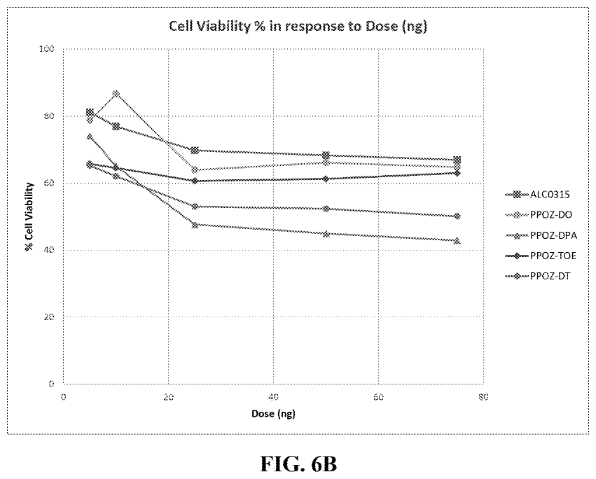

Human Embryonic Kidney (HEK) 293 cells (ATCC) were seeded in a white 96 well CulturPlate™-96 (Revity) at a density of 25,000 cells/well in 100 μL of DMEM media (Corning) with 10% FBS (Corning). The HEK293 plate was incubated at 37° C./5% CO₂ overnight. Each LNP formulation was diluted in 25 mM Tris HCl pH 7 at dose levels of 0.75, 0.5, 0.25, 0.1, and 0.05 ng/μL based on the eGFP saRNA payload concentration. 100 μL of each dose was added to the plate. Tris-HCl buffer (100 μL) was used as a blank control for cell viability calculations. The plate was returned to the incubator to allow for cellular uptake of the formulations. After overnight incubation, the plate was assayed for cell viability using the Cell Titer-Glo Luminescent Cell Viability Assay Kit (Promega). As shown in FIGS. 6A-6B, the luminescent signal (RLU) was recorded using the Varioskan LUX plate reader:

$$\% Cell Viability was calculated by : \frac{dose response RLU}{blank RLU} * 100$$

### Example 5: In Vitro Transfection of HEK293 Cells with EGFP saRNA-LNPs

LNP lipid stocks were prepared in ethanol at selected molar ratios of PPOZ 2.6K-OH 2.6k NMPCA-Lipids, 1,2-distearoyl-sn-glycero-3-phosphocholine (DSPC, Bachem 4005619), cholesterol (VWR 0433) and PEOZ 2K DMA (as described in U.S. Pat. No. 12,233,132) in EtOH at a lipid stock concentration of 2 mg/mL. The saRNA aqueous stock was hand-mixed with the lipid stocks at a 3:1 ratio and diluting to a 0.75 ng/μL saRNA concentration with 25 mM Tris, pH 7.5. The resulting mixture was incubated for one hour at room temperature to reduce ethanol content in the lipid nanoparticles.

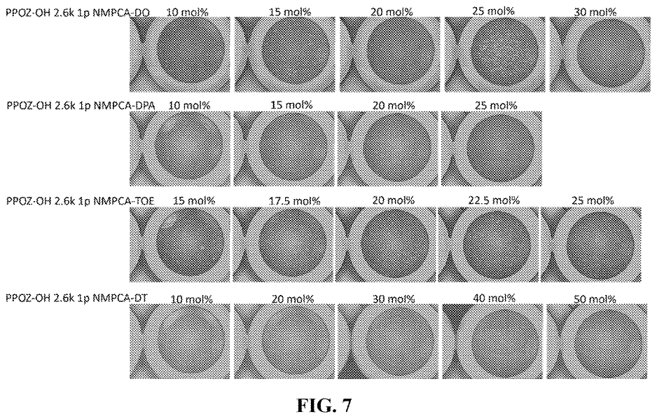

Human Embryonic Kidney (HEK) 293 cells (ATCC) were seeded in a 96 well cell treated plate (Corning) at a density of 50,000 cells/well in 100 μL of DMEM media (Corning) with 10% FBS (Corning). The HEK293 plate was incubated at 37° C./5% CO₂ overnight to a confluency of about 50 to 60%. Following incubation, each well was treated with LNPs at a 50 ng EGFP saRNA concentration. Media was removed from each well, and replaced with 100 μL Opti-MEM, reduced serum medium (Gibco). The plate was returned to the incubator to allow for cellular uptake of the formulations. After 3-4 hours, the wells containing Opti-MEM were replaced with 100 uL DMEM with 10% FBS and the plate was returned to incubator for 24 hours. Cells were analyzed on an EVOS-M7000 microscope (Thermoscientific) for cellular vitality and expression of enhanced Green Fluorescent Protein (eGFP) signal. As demonstrated in FIG. 7, the transfection of saRNA-LNPs yielded a positive green signal indicating cellular uptake of LNPs and expression of eGFP.

### Example 6: In Vitro Transfection of HEK293 Cells with FLuc saRNA LNP

The self-amplifying RNA (saRNA) was the luciferase protein encoded with the luciferase gene from the firefly, Photinus pyralis (Creative Biogene, PMSAR-0002). The single-stranded RNA has 9,461 nucleotides, and the firefly luciferase has a molecular weight of 62 kDa. The supplied solution of 1.109 μg/μL in water, was diluted with 20 mM sodium acetate buffer, pH 4.5, to prepare a solution of 0.01 μg/μL concentration.

LNP lipid stocks were prepared in ethanol at selected molar ratios of PPOZ 2.6K-OH 2.6k NMPCA-Lipids, 1,2-distearoyl-sn-glycero-3-phosphocholine (DSPC, Bachem 4005619), cholesterol (VWR 0433) and PEOZ 2K DMA (as described in U.S. Pat. No. 12,233,132) in EtOH at a lipid stock concentration of 2 mg/m. The saRNA aqueous stock was hand-mixed with the lipid stocks at a 3:1 ratio and diluting to a 0.75 ng/μL saRNA concentration with 25 mM Tris, pH 7.5. The resulting mixture was incubated for one hour at room temperature to reduce ethanol content in the lipid nanoparticles.

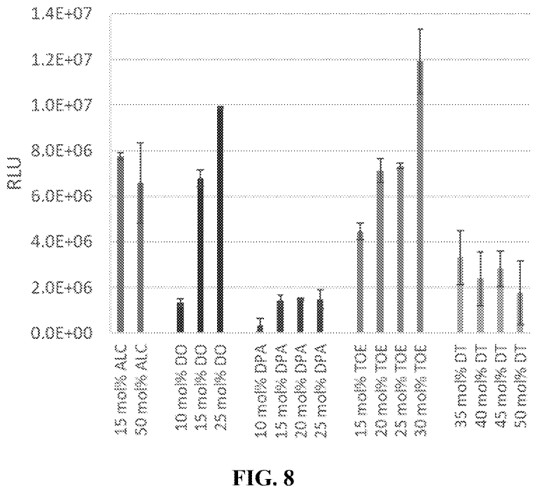

Human Embryonic Kidney (HEK) 293 cells (ATCC) were seeded in a 96 well cell treated plate (Corning) at a density of 50,000 cells/well in 100 μL of DMEM media (Corning) with 10% FBS (Corning). The HEK293 plate was incubated at 37° C./5% CO₂ overnight to a confluency of about 50 to 60%. Following incubation, each well was treated with LNPs at a 50 ng FLuc saRNA concentration. Media was removed from each well, and replaced with 100 μL Opti-MEM, reduced serum medium (Gibco). The plate was returned to the incubator to allow for cellular uptake of the formulations. After 3 to 4 hours, the wells containing Opti-MEM were replaced with 100 uL DMEM with 10% FBS and the plate was returned to incubator for 24 hours. Media was removed from each well and replaced with 100 uL of luciferase reagent (Promega, Bright-Glo™ Luciferase Assay System). The plate was left to incubate for 5 min at room temperature. The luminescence of the plates was read using a Varioskan LUX (Invitrogen) (FIG. 8).

### Example 7. Synthesis of DO-PPOZ 500-IGs (where IG=Ionizable Group)

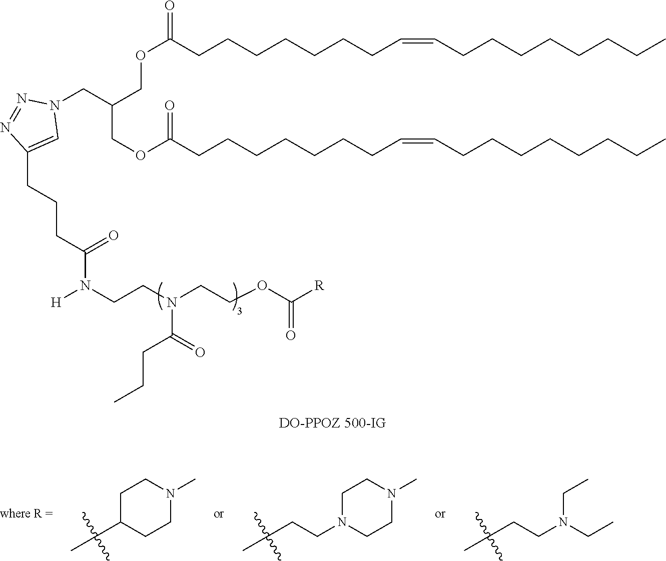

### Step 1. Synthesis of Ptyn-PPOZ-OH 500

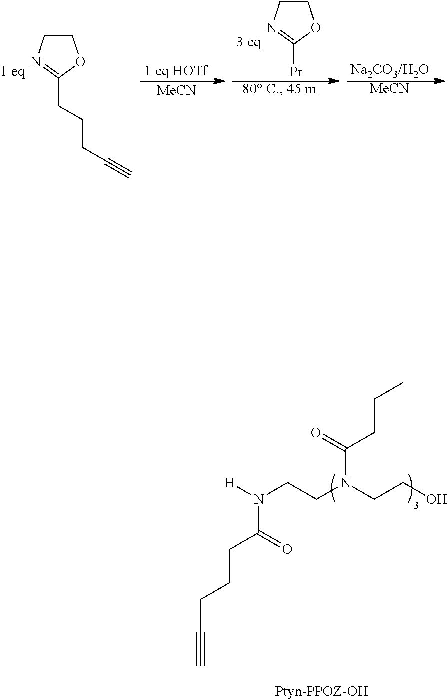

An oven-dried 50 mL round-bottomed flask was charged with 2-(5-pentynyl)-2-oxazoline (0.405 g, 2.95 mmol, 1.0 equiv) followed by acetonitrile (6 mL) and a stir bar under an atmosphere of argon. TfOH (0.261 mL, 2.95 mmol, 1.0 equiv) was added dropwise, and the mixture was allowed to stir at room temperature for 5 minutes. Following this time period, n-propyl-2-oxazoline (1.00 g, 8.85 mmol, 3 equiv) was added, and the resulting mixture was heated to 80° C. and stirred for 45 minutes. The polymerization mixture was then cooled to room temperature and diluted using MeCN (24 mL). An aqueous solution of Na₂CO₃ (1.56 g, 14.7 mmol, 5.0 equiv) in H₂O (15 mL) was added. The resulting mixture was allowed to stir for at least 12 hours. Following this time period, most volatiles were removed using a rotary evaporator and the resulting mixture was extracted twice with CH₂Cl₂ (25 mL each). The combined organic phases were dried over Na₂SO₄, filtered, concentrated, and further dried in vacuo to provide 1.38 g of the desired product.

¹H NMR analysis showed the standard backbone signals for PPOZ (500 MHz, DMSO-d₆) δ 0.84 (m, 3H, —C(═O)CH₂CH₂CH₃), 1.48 (m, 2H, —C(═O)CH₂CH₂CH₃), 2.27 and 2.21 (m, total area 2H, —C(═O)CH₂CH₂CH₃), 3.20 (m, 2H, —N—CH₂), 3.34 (m, 4H, —NCH₂CH₂N— backbone), and 7.94 (br m, 1H, terminal NH). Signals for the pendant group were present at δ 1.65 ppm (—CH₂CH₂CH₂C≡CH) and δ2.77 ppm (—C≡CH). The number of pendant groups was determined from the ¹H NMR analysis to be 1.0 by comparing the integrations of pendant group protons and polymer backbone protons, and the MALDI-TOF gave Mn 494 Da (Calcd Mn 516 Da).

### Step 2. Synthesis of Ptyn-PPOZ 500-IGs

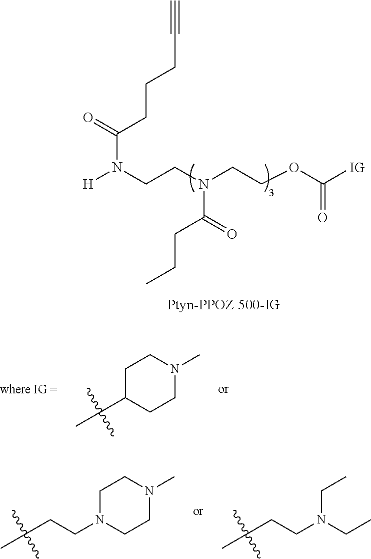

### Step 2A. Synthesis of Ptyn-PPOZ 500-NMP

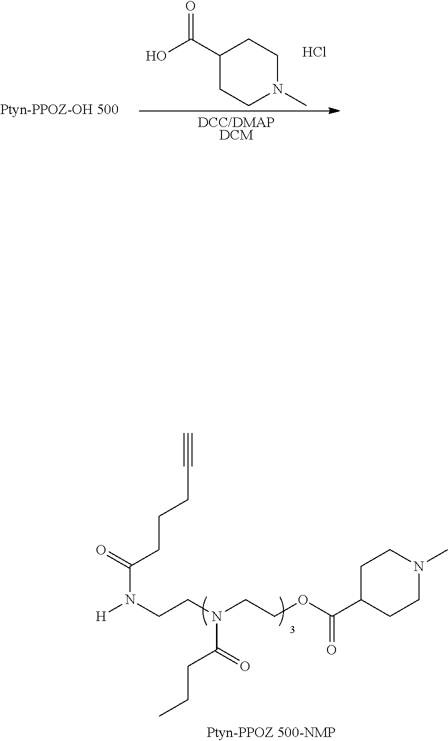

1-methylpiperidine-4-carboxylic acid HCl (0.162 g, 0.900 mmol, 1.1 equiv), Ptyn-PPOZ-OH 500 (0.405 g, 0.818 mmol, 1.0 equiv), DMAP (0.020 g, 0.164 mmol, 0.2 equiv), and anhydrous DCM (8 mL) were added to a 25-mL flask. After stirring for 10 minutes, DCC (0.186 g, 0.900 mmol, 1.1 equiv) was added and the reaction mixture was allowed to stir overnight under an atmosphere of argon. The mixture was filtered using a syringe filter, and the filtrate was concentrated. The residue was purified by reverse phase C18 column chromatography using water and methanol as the eluents to provide the title compound (0.242 g, 42.8% yield).

¹H NMR (Varian, 500 MHz, 10 mg/mL DMSO-d₆, δ) analysis showed the standard backbone signals for PPOZ at 0.83 (m, 3H, —C(═O)CH₂CH₂CH₃), 1.48 (m, 2H, —C(═O)CH₂CH₂CH₃), 2.21 and 2.27 (m, total area 2H, —C(═O)CH₂CH₂CH₃), 3.16 (m, 2H, —N—CH₂), 3.34 (m, 4H, —NCH₂CH₂N— backbone), and 7.91 (br m, 1H, terminal NH). The attachment of N-methyl piperidinyl group were proved by the proton signals at δ 1.95 (m, 1H, pip-CH—C(O)O—), 2.01 (m, 2H, -piperidyl (CHH)N—CH₃), 2.15 (br s, 3H, pip-N—CH₃), 2.58 (m, total area 6H, piperidyl), 4.15 and 4.20 (br s, 2H, —CH₂—O(C═O)—). The MALDI-TOF gave Mn 649 Da (Calcd Mn 640 Da).

### Step 2B. Synthesis of Ptyn-PPOZ 500-MPZ

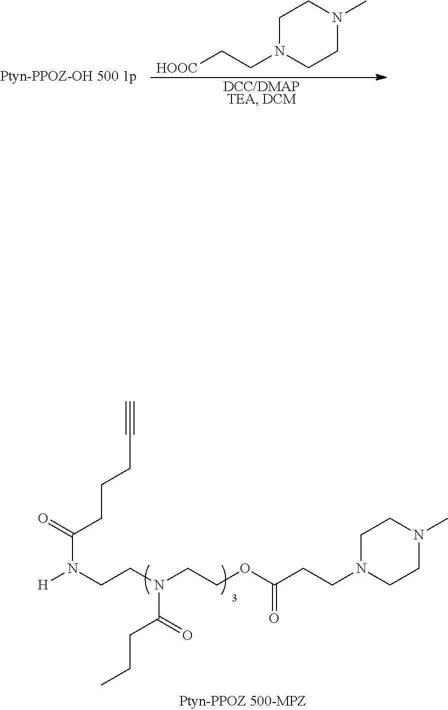

Ptyn-PPOZ 500-MPZ was prepared in an identical fashion to that described for Ptyn-PPOZ 500-NMP using 3-(4-methylpiperazin-1-yl) propanoic acid (67.8 mg, 0.386 mmol, 1.5 equiv), Ptyn-PPOZ-OH 500 (127.4 mg, 0.257 mmol, 1.0 equiv), DMAP (6.29 mg, 0.0515 mmol, 0.2 equiv), DCC (79.6 mg, 0.386 mmol, 1.5 equiv), TEA (53.8 μL, 0.386 mmol, 1.5 equiv), and anhydrous DCM (2 mL). The crude product was purified by reverse phase C18 column chromatography using water and methanol as the eluents to provide the title compound (97.4 mg, 50.0% yield).

¹H NMR (Varian, 500 MHz, 10 mg/mL DMSO-d₆, δ) analysis showed the standard backbone signals for PPOZ at 0.83 (m, 3H, —C(═O)CH₂CH₂CH₃), 1.48 (m, 2H, —C(═O)CH₂CH₂CH₃), 2.21 and 2.27 (m, total area 2H, —C(═O)CH₂CH₂CH₃), 3.16 (m, 2H, —N—CH₂), 3.34 (m, 4H, —NCH₂CH₂N— backbone), and 7.94 (br m, 1H, terminal NH). Additionally, signals for the pendant group were present at δ 1.65 (—CH₂CH₂CH₂C≡CH) and δ 2.78 (—C≡CH). The attachment of N-methyl piperazinyl group was proved by the proton signals at δ 2.30 (br s, 3H, PZ—N—CH₃), 2.45 (m, total area 8H, piperazinyl), 2.55 (m, 4H, —C(═O)CH₂CH₂N—PZ), 4.10 and 4.15 (br s, 2H, —CH₂—O(C═O)—). The MALDI-TOF gave Mn 670.4 Da (Calcd Mn 649 Da).

### Step 2C. Synthesis of Ptyn-PPOZ 500-DEA

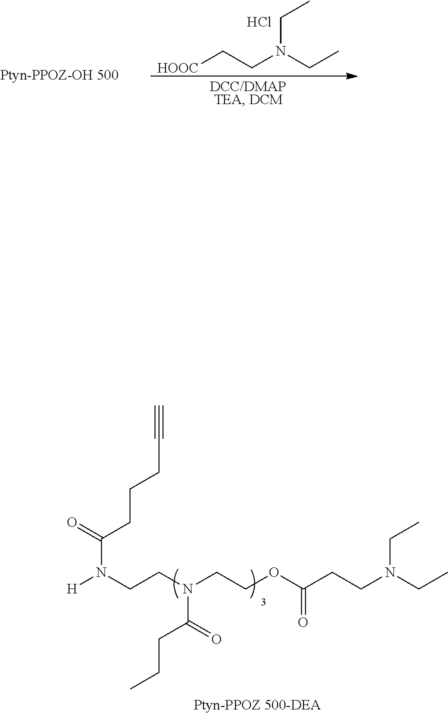

Ptyn-PPOZ 500-DEA was prepared in an identical fashion to that described for Ptyn-PPOZ 500-NMP using 3-(diethylamino)propionic acid HCl (148.6 mg, 0.818 mmol, 3.0 equiv), Ptyn-PPOZ-OH 500 (135.0 mg, 0.273 mmol, 1.0 equiv), DMAP (6.66 mg, 0.0545 mmol, 0.2 equiv), DCC (168.8 mg, 0.818 mmol, 3 equiv), TEA (114.0 μL, 0.818 mmol, 3.0 equiv), and anhydrous DCM (3 mL). The crude product was purified by reverse-phase C18 column chromatography using water and methanol as the eluents, affording the title compound (113.4 mg, 64.1% yield).

¹H NMR (Varian, 500 MHz, 10 mg/mL DMSO-d₆, δ) analysis showed the standard backbone signals for PPOZ at 0.83 (m, 3H, —C(═O)CH₂CH₂CH₃), 1.48 (m, 2H, —C(═O)CH₂CH₂CH₃), 2.21 and 2.27 (m, total area 2H, —C(═O)CH₂CH₂CH₃), 3.16 (m, 2H, —N—CH₂), 3.34 (m, 4H, —NCH₂CH₂N— backbone), and 7.94 (br m, 1H, terminal NH). Additionally, signals for the pendant group were present at δ 1.65 (—CH₂CH₂CH₂C≡CH) and δ 2.78 (—C≡CH). The attachment of diethylamino propyl group was proved by the proton signals at δ 1.19 (t, 6H, —N(CH₂CH₃)₂), 3.18 (m, 4H, —N(CH₂CH₃)₂), 4.17 and 4.20 (br s, 2H, —CH₂—O(C═O)—). The MALDI-TOF gave Mn 622.2 Da (Calcd Mn 649 Da).

### Step 3. Synthesis of DO-PPOZ 500-IGs

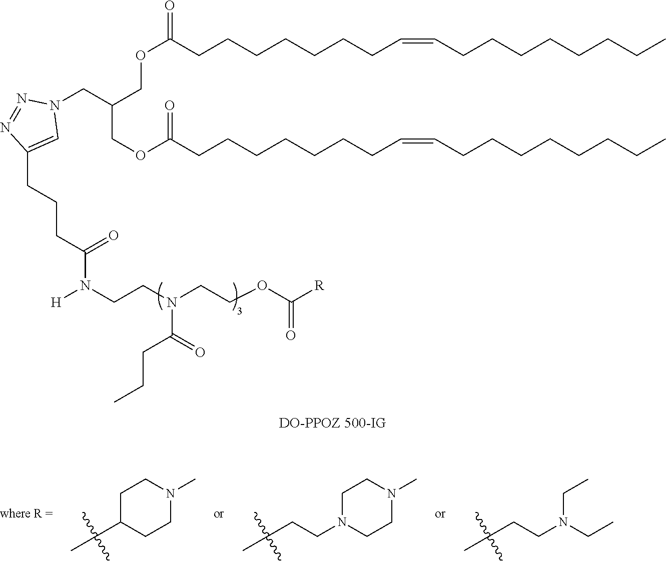

### Step 3A. Synthesis of DO-PPOZ 500-NMP

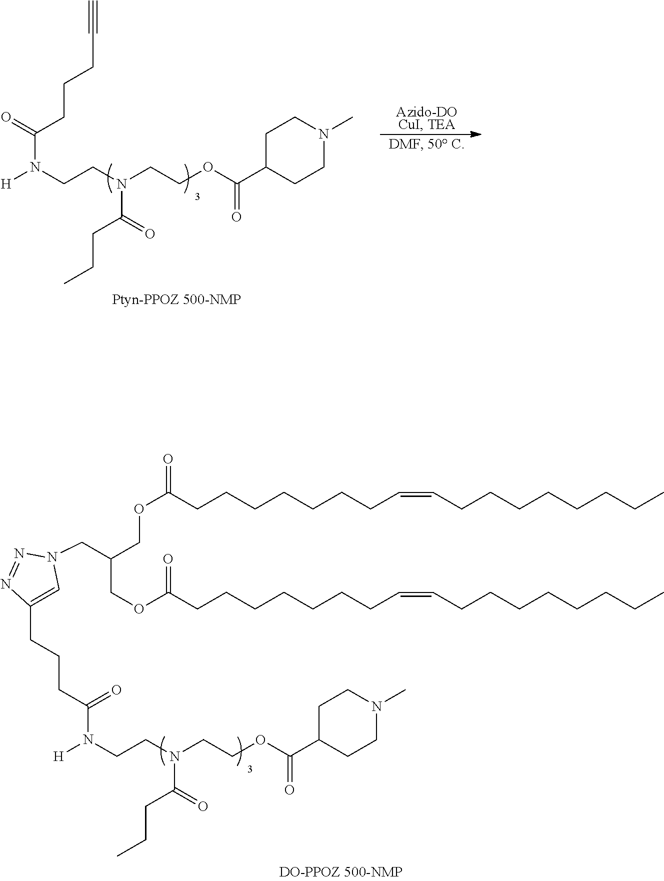

An oven-dried 25-mL round bottomed flask, equipped with a reflux condenser and an argon gas inlet, was charged with Ptyn-PPOZ 500-NMP (54.4 mg, 0.0850 mmol, 1.0 equiv, Mn 640 Da) and 2-(azidomethyl)propane-1,3-diyl dioleate (azido-DO, 56.1 mg, 0.0850 mmol, 1.0 equiv) followed by a stir bar and THF (1.6 mL) under an argon atmosphere. After addition of CuI (14.1 mg, 0.0740 mmol, 0.87 equiv) and TEA (118.5 μL, 0.850 mmol, 10 eq), the resulting mixture was stirred for 5 minutes at room temperature and then allowed to stir for 18 hours at 50° C. After cooling down to room temperature, the reaction mixture was quenched by adding 1 N aqueous HCl (0.9 mL) followed by stirring for 5 minutes. The mixture was passed through the Dowex® M4195 column and the column was eluted with a mixture of THF and 2 mN HCl (3:1). The resulting solution was concentrated using a rotary evaporator at 34° C. The residue was purified by silica-gel column chromatography using a Biotage (Sfar C18 cartridge), with a mixture of water and methanol as an eluting solvent. After removing MeOH, the resulting aqueous solution was freeze-dried using a lyophilizer to provide the desired product (36.3 mg, 33.2% yield) as a pale yellow sticky powder.

¹H NMR (Varian, 500 MHz, 10 mg/mL DMSO-d₆, δ) analysis showed the standard backbone signals for PPOZ at 0.84 (m, 3H, —C(═O)CH₂CH₂CH₃), 1.50 (m, 2H, —C(═O)CH₂CH₂CH₃), 2.21 and 2.27 (m, total area 2H, —C(═O)CH₂CH₂CH₃), 3.19 (m, 2H, —N—CH₂), 3.34 (m, 4H, —NCH₂CH₂N— backbone), and 7.91 (br m, 1H, terminal NH). Signals for the N-methyl piperidinyl group were present at δ 1.95 (m, 1H, pip-CH—C(O)O—), 2.00 (m, 2H, -piperidyl (CHH)N—CH₃), 2.27 (br s, 3H, pip-N—CH₃), 2.58 (m, total area 6H, piperidyl), 4.17 (m, 2H, —CH₂—O(C═O)—). The attachment of azido lipid (N₃-DO) was proved by the proton signals at δ 0.84 (m, 6H, —(CH₂)₆CH₃), 1.25 (m, 40H, —(CH₂)₄CH₂ CH═CHCH₂(CH₂)₆CH₃), 1.64 (m, 4H, —OC(═O)CH₂CH₂—), 2.00 (m, 8H, —CH₂CH═CHCH₂—), 2.26 (m, 1H, CH[(CH₂)₃]), 2.35 (m, 4H, —OC(═O)CH₂—), 4.02 (m, 4H, —CH[(CH₂)O-]₂), 4.42 (m, 2H, triazole N—CH₂—), 5.32 (m, 4H, —CH₂CH═CHCH₂—), 7.89 (br s, 1H, triazole ring, resulted by ‘Click’ reaction). Additionally, one of the pendant group peaks appears at δ 1.77 (m, 2H, backbone-CH₂CH₂CH₂-triazole ring, due to the click). The number of pendant groups was determined from the ¹H NMR analysis to be 1.0 for the NMP moiety and 1.0 for the DO lipid moiety by comparing the integrations of pendant group protons and polymer backbone protons, and the MALDI-TOF gave Mn 1434 Da (Calcd Mn 1300 Da).

### Step 3B. Synthesis of DO-PPOZ 500-MPZ

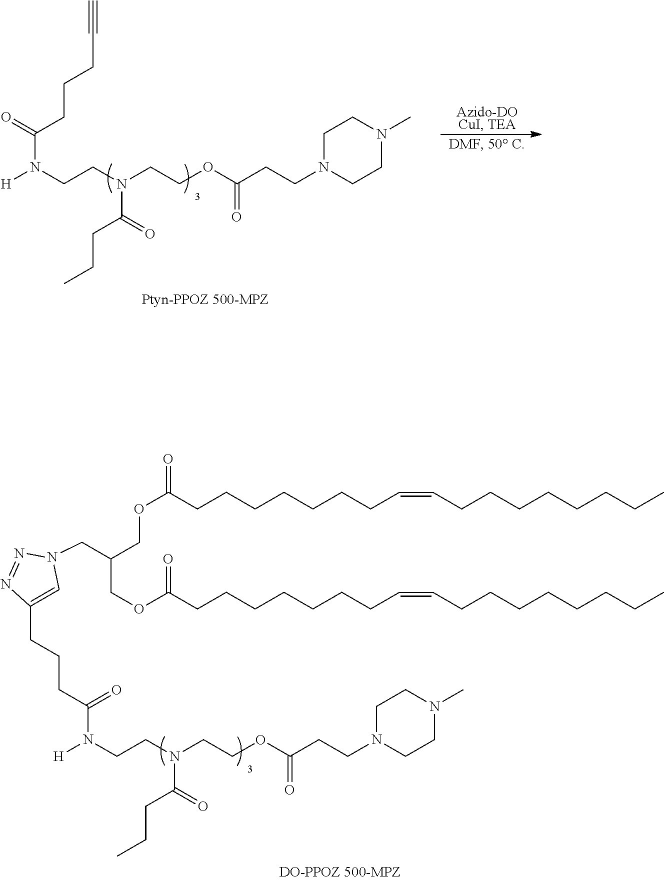

DO-PPOZ 500-MPZ was prepared in an identical fashion to that described for DO-PPOZ 500-NMP using Ptyn-PPOZ 500-MPZ (67.7 mg, 0.101 mmol, 1.0 equiv), 2-(azidomethyl)propane-1,3-diyl dioleate (azido-DO, 80.0 mg, 0.121 mmol, 1.2 equiv), CuI (7.69 mg, 0.0404 mmol, 0.4 equiv), TEA (98.5 μL, 0.707 mmol, 7 eq), and DMF (2.5 mL) under an argon atmosphere. The crude product was purified by reverse-phase C18 column chromatography using water and methanol as the eluents, affording the title compound (25.4 mg, 25.1% yield).

¹H NMR (Varian, 500 MHz, 10 mg/mL DMSO-d₆, δ) analysis showed the standard backbone signals for PPOZ at 0.85 (m, 3H, —C(═O)CH₂CH₂CH₃), 1.50 (m, 2H, —C(═O)CH₂CH₂CH₃), 2.21 and 2.27 (m, total area 2H, —C(═O)CH₂CH₂CH₃), 3.16 (m, 2H, —N—CH₂), 3.34 (m, 4H, —NCH₂CH₂N— backbone), and 7.94 (br m, 1H, terminal NH). Signals for the N-methyl piperazinyl group were present at δ 2.30 (br s, 3H, PZ—N—CH₃), 2.45 (m, total area 8H, piperazinyl), 2.55 (m, 4H, —C(═O)CH₂CH₂N—PZ), 4.10 and 4.15 (br s, 2H, —CH₂—O(C═O)—). )—). The attachment of azido lipid (N₃-DO) was proved by the proton signals at δ 0.85 (m, 6H, —(CH₂)₆CH₃), 1.25 (m, 40H, —(CH₂)₄CH₂ CH═CHCH₂(CH₂)₆CH₃), 1.64 (m, 4H, —OC(═O)CH₂CH₂—), 2.00 (m, 8H, —CH₂CH═CHCH₂—), 2.26 (m, 1H, CH[(CH₂)₃]), 2.35 (m, 4H, —OC(═O)CH₂—), 4.02 (m, 4H, —CH[(CH₂)O-]₂), 4.42 (m, 2H, triazole N—CH₂—), 5.32 (m, 4H, —CH₂CH═CHCH₂—), 7.89 (br s, 1H, triazole ring, resulted by ‘Click’ reaction). In addition, one of the pendant group peaks appears at δ 1.77 (m, 2H, backbone-CH₂CH₂CH₂-triazole ring, due to the click). The number of pendant groups was determined from the ¹H NMR analysis to be 1.0 for the MPZ moiety and 1.0 for the DO lipid moiety by comparing the integrations of pendant group protons and polymer backbone protons, and the MALDI-TOF gave Mn 1262.6 Da (Calcd Mn 1309 Da).

### Step 3C. Synthesis of DO-PPOZ 500-DEA

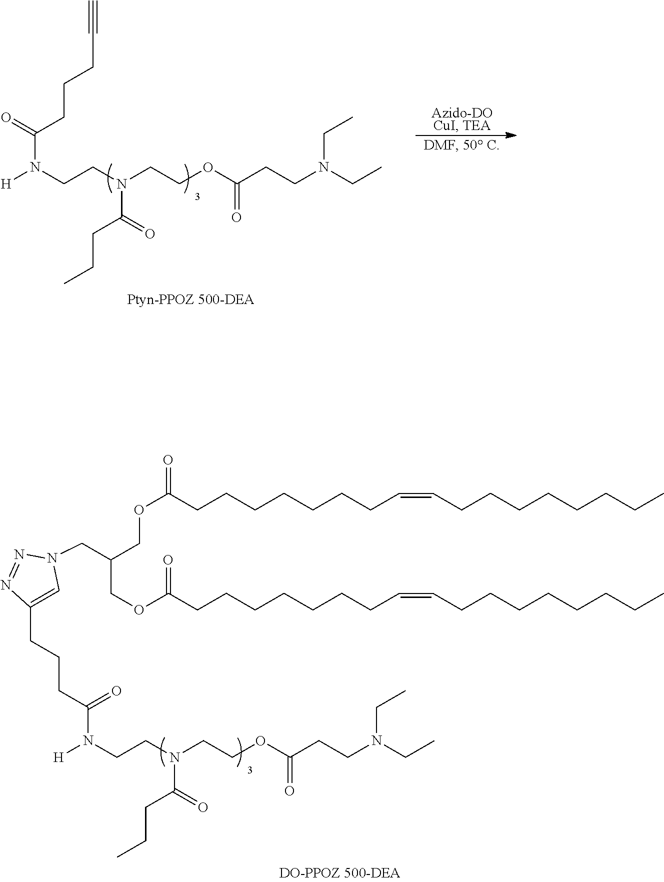

DO-PPOZ 500-DEA was prepared in an identical fashion to that described for DO-PPOZ 500-NMP using Ptyn-PPOZ 500-DEA (86.9 mg, 0.140 mmol, 1.0 equiv), 2-(azidomethyl)propane-1,3-diyl dioleate (azido-DO, 92.2 mg, 0.140 mmol, 1.0 equiv), CuI (18.0 mg, 0.0944 mmol, 0.68 equiv), TEA (98.4 μL, 0.706 mmol, 5 eq), and DMF (2.0 mL) under an argon atmosphere. The crude product was purified by reverse-phase C18 column chromatography using water and methanol as the eluents, affording the title compound (17.8 mg, 10.0% yield).

¹H NMR (Varian, 500 MHz, 10 mg/mL DMSO-d₆, δ) analysis showed the standard backbone signals for PPOZ at 0.84 (m, 3H, —C(═O)CH₂CH₂CH₃), 1.50 (m, 2H, —C(═O)CH₂CH₂CH₃), 2.21 and 2.27 (m, total area 2H, —C(═O)CH₂CH₂CH₃), 3.16 (m, 2H, —N—CH₂), 3.34 (m, 4H, —NCH₂CH₂N— backbone), and 7.94 (br m, 1H, terminal NH). Signals for the diethylamino propyl group were present signals at δ 1.19 (t, 6H, —N(CH₂CH₃)₂), 3.15 (m, 4H, —N(CH₂CH₃)₂), and 4.17 and 4.21 (br s, 2H, —CH₂—O(C═O)—). The attachment of azido lipid (N₃-DO) was proved by the proton signals at δ 0.84 (m, 6H, —(CH₂)₆CH₃), 1.25 (m, 40H, —(CH₂)₄CH₂ CH═CHCH₂(CH₂)₆CH₃), 1.64 (m, 4H, —OC(═O)CH₂CH₂—), 2.00 (m, 8H, —CH₂CH═CHCH₂—), 2.26 (m, 1H, CH[(CH₂)₃]), 2.35 (m, 4H, —OC(═O)CH₂—), 4.00 (m, 4H, —CH[(CH₂)O-]₂), 4.41 (m, 2H, triazole N—CH₂—), 5.32 (m, 4H, —CH₂CH═CHCH₂—), 7.89 (br s, 1H, triazole ring, resulted by ‘Click’ reaction). In addition, one of the pendant group peaks appears at δ 1.81 (m, 2H, backbone-CH₂CH₂CH₂-triazole ring, due to the click). The number of pendant groups was determined from the ¹H NMR analysis to be 1.0 for the DEA moiety and 1.0 for the DO lipid moiety by comparing the integrations of pendant group protons and polymer backbone protons, and the MALDI-TOF gave Mn 1227.0 Da (Calcd Mn 1282 Da).

### Example 8. Synthesis of DO-PEOZ 700-IGs (where, IG=Ionizable Group)

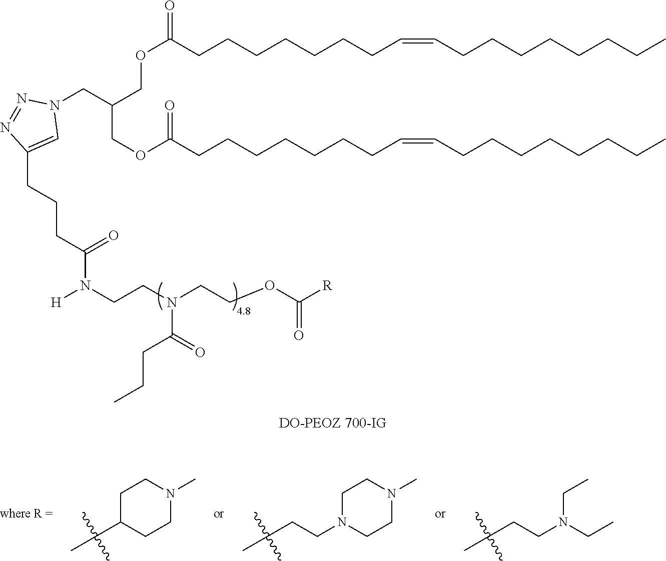

### Step 1. Synthesis of Ptyn-PEOZ-OH 700 Da

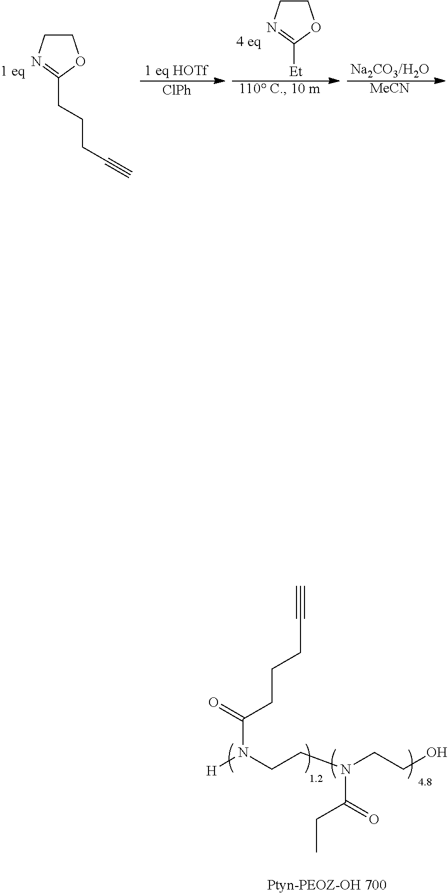

An oven-dried 50 mL round-bottomed flask was charged with 2-(5-pentynyl)-2-oxazoline (0.686 g, 5.00 mmol, 1.0 equiv) followed by chlorobenzene (12.5 mL) and a stir bar under an atmosphere of argon. TfOH (0.261 mL, 2.95 mmol, 1.0 equiv) was added dropwise at 0° C., and the mixture was allowed to stir at room temperature for 45 minutes. Following this time period, 2-ethyl-2-oxazoline (1.98 g, 20.00 mmol, 4 equiv) was added, and the resulting mixture was stirred at 110° C. for 10 minutes. The polymerization mixture was then cooled to room temperature and chlorobenzene was removed. The residue was dissolved in MeCN (80 mL) and an aqueous solution of Na₂CO₃ (2.12 g, 20.0 mmol, 4.0 equiv) in H₂O (40 mL) was added. The resulting mixture was allowed to stir for at least 12 hours. Following this time period, most volatiles were removed using a rotary evaporator and the resulting mixture was extracted twice with CH₂Cl₂ (40 mL each). The combined organic phases were dried over Na₂SO₄, filtered, and concentrated. The residue was purified by reverse phase column chromatography using a Biotage (SNAP Ultra C18 cartridge), with a mixture of water and methanol as the eluents. After removing MeOH, the resulting aqueous solution was freeze-dried to provide 1.47 g of the desired product.

¹H NMR analysis showed the standard backbone signals for PEOZ (500 MHz, DMSO-d₆) δ 7.92 (br m, 1H, terminal NH), 3.33 (m, 4H, —NCH₂CH₂N— backbone), 3.20 (m, 2H, —N—CH₂), 2.34 (m, 2H, —C(═O)CH₂CH₃), and 0.97 (m, 3H, —C(═O)CH₂CH₃). Signals for the pendant group were present at δ 2.77 ppm (—C≡CH) and δ 1.66 ppm (—CH₂CH₂CH₂C≡CH). The ratio of PtynOZ:EOZ was determined as 1.2:4.8 from the ¹H NMR analysis by comparing the integrations of pendant group protons and polymer backbone protons, and the molecular weight (Mn) was determined as 668 Da by MALDI-TOF analysis.

### Step 2. Synthesis of Ptyn-PEOZ 700-IGs

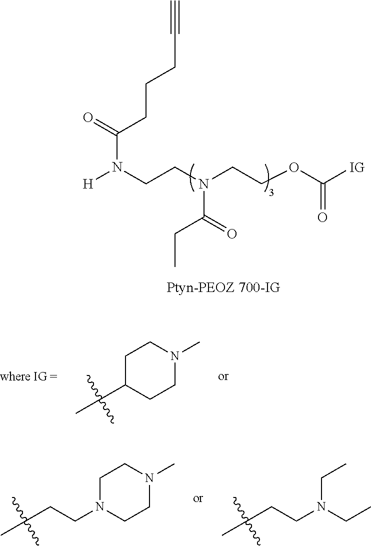

### Step 2A. Synthesis of Ptyn-PEOZ 700-NMP

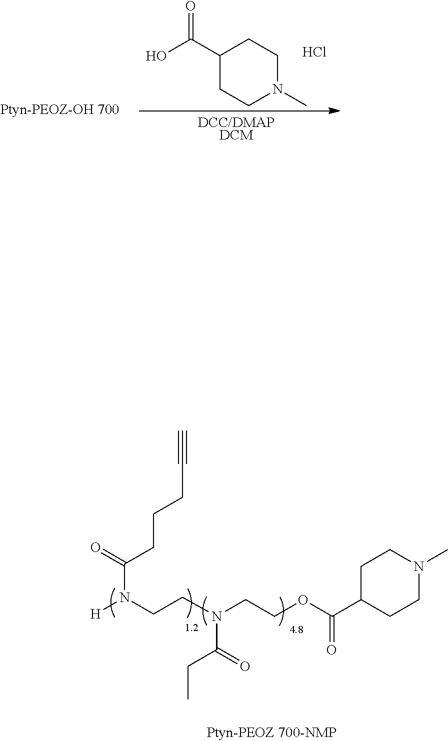

Ptyn-PEOZ 700-NMP was prepared in an analogous fashion to that described for Ptyn-PPOZ 500-NMP using 1-methylpiperidine-4-carboxylic acid HCl (100.8 mg, 0.561 mmol, 1.15 equiv), Ptyn-PEOZ-OH 700 (372.2 mg, 0.490 mmol, 1.0 equiv), DMAP (9.68 mg, 0.0792 mmol, 0.16 equiv), DCC (115.7 mg, 0.561 mmol, 1.15 equiv), TEA (78.2 μL, 0.561 mmol, 1.15 equiv), and anhydrous DCM (4 mL). The crude product was purified by reverse-phase C18 column chromatography using water and methanol as the eluents, affording the title compound (80.0 mg, 37.0% yield).

Besides the standard polymer backbone peaks, ¹H NMR (Varian, 500 MHz, 10 mg/mL DMSO-d₆, δ) analysis showed signals for the N-methyl piperidinyl group were present at δ 1.95 (m, 1H, pip-CH—C(O)O—), 2.01 (m, 2H, -piperidyl (CHH)N—CH₃), 2.15 (br s, 3H, pip-N—CH₃), 2.58 (m, total area 6H, piperidyl), 4.15 and 4.20 (br s, 2H, —CH₂—O(C═O)—). Additionally, signals for the pendant group were present at δ 1.65 (—CH₂CH₂CH₂C≡CH) and δ 2.78 (—C≡CH). The MALDI-TOF gave Mn 879 Da (Calcd Mn 776 Da).

### Step 2B. Synthesis of Ptyn-PEOZ 700-MPZ

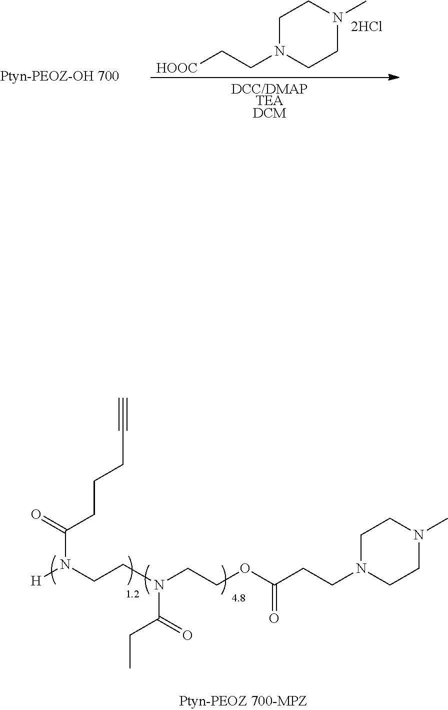

Ptyn-PEOZ 700-MPZ was prepared in an analogous fashion to that described for Ptyn-PPOZ 500-NMP using 3-(4-methylpiperazin-1-yl) propanoic acid (80.2 mg, 0.311 mmol, 1.2 equiv), Ptyn-PEOZ-OH 700 (172.2 mg, 0.259 mmol, 1.0 equiv), DMAP (6.33 mg, 0.0518 mmol, 0.2 equiv), DCC (64.8 mg, 0.311 mmol, 1.2 equiv), TEA (86.6 μL, 0.621 mmol, 2.4 equiv), and anhydrous DCM (4 mL). The crude product was purified by reverse phase C18 column chromatography using water and methanol as the eluents to provide the title compound (198 mg, 96.1% yield).

Besides the standard polymer backbone peaks, ¹H NMR (Varian, 500 MHz, 10 mg/mL DMSO-d₆, δ) analysis showed signals for the N-methyl piperazinyl group were present at δ 2.30 (br s, 3H, PZ—N—CH₃), 2.45 (m, total area 8H, piperazinyl), 2.55 (m, 4H, —C(═O)CH₂CH₂N—PZ), 4.10 and 4.15 (br s, 2H, —CH₂—O(C═O)—). Additionally, signals for the pendant group were present at δ 1.65 (—CH₂CH₂CH₂C≡CH) and δ 2.78 (—C≡CH). The MALDI-TOF gave Mn 863 Da (Calcd Mn 822 Da).

### Step 2C. Synthesis of Ptyn-PEOZ 700-DEA

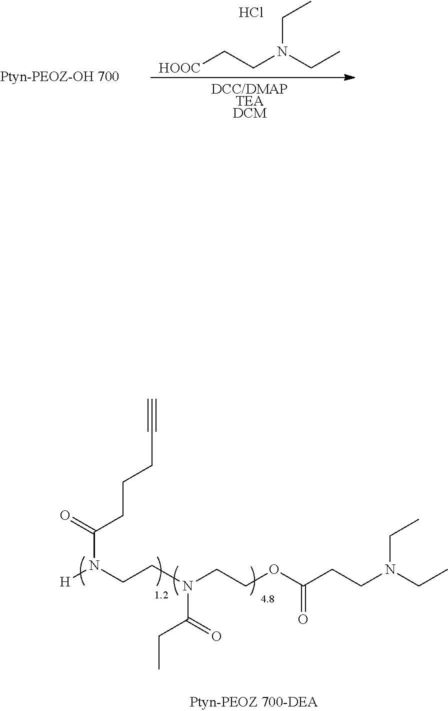

Ptyn-PEOZ 700-DEA was prepared in an analogous fashion to that described for Ptyn-PPOZ 500-NMP using 3-(diethylamino)propionic acid HCl (115.9 mg, 0.626 mmol, 2.3 equiv), Ptyn-PEOZ-OH 700 (182.5 mg, 0.273 mmol, 1.0 equiv), DMAP (5.09 mg, 0.0417 mmol, 0.15 equiv), DCC (129.1 mg, 0.626 mmol, 2.3 equiv), TEA (87.2 μL, 0.626 mmol, 2.3 equiv), and anhydrous DCM (4 mL). The crude product was purified by reverse-phase C18 column chromatography using water and methanol as the eluents, affording the title compound (120 mg, 57.4% yield).

Besides the standard polymer backbone peaks, ¹H NMR (Varian, 500 MHz, 10 mg/mL DMSO-d₆, δ) analysis showed signals for the diethylamino ethyl group were present at δ 1.19 (t, 6H, —N(CH₂CH₃)₂), 3.18 (m, 4H, —N(CH₂CH₃)₂), 4.17 and 4.20 (br s, 2H, —CH₂—O(C═O)—). Additionally, signals for the pendant group were present at δ 1.65 (—CH₂CH₂CH₂C≡CH) and δ 2.78 (—C≡CH). The MALDI-TOF gave Mn 765 Da (Calcd Mn 795 Da).

### Step 3. Synthesis of DO-PEOZ 700-IGs

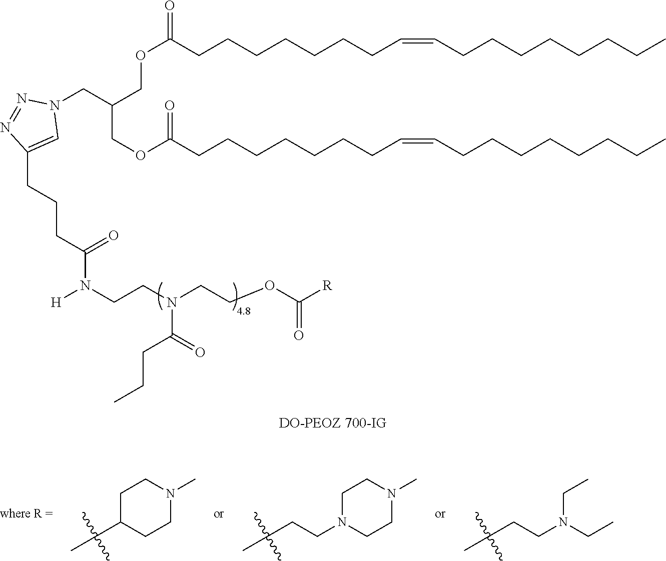

### Step 3A. Synthesis of DO-PEOZ 700-NMP

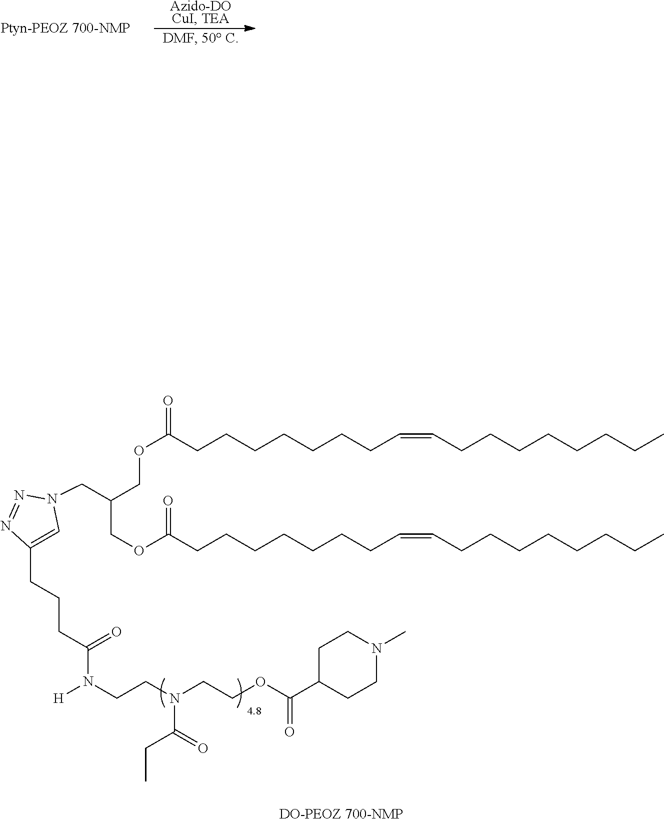

DO-PEOZ 700-NMP was prepared in an analogous fashion to that described for DO-PPOZ 500-NMP using Ptyn-PEOZ 700-NMP (62.1 mg, 0.0707 mmol, 1.0 equiv), 2-(azidomethyl)propane-1,3-diyl dioleate (azido-DO, 65.3 mg, 0.0989 mmol, 1.4 equiv), CuI (9.48 mg, 0.04978 mmol, 0.70 equiv), TEA (74.2 μL, 0.532 mmol, 7.5 eq), and DMF (2.0 mL) under an argon atmosphere. The crude product was purified by reverse-phase C18 column chromatography using water and methanol as the eluents, affording the title compound (30.0 mg, 31.4% yield).

Besides the standard polymer backbone peaks, ¹H NMR (Varian, 500 MHz, 10 mg/mL DMSO-d₆, δ) analysis showed signals for the N-methyl piperidinyl group were present at δ 1.95 (m, 1H, pip-CH—C(O)O—), 2.01 (m, 2H, -piperidyl (CHH)N—CH₃), 2.15 (br s, 3H, pip-N—CH₃), 2.58 (m, total area 6H, piperidyl), 4.15 and 4.20 (br s, 2H, —CH₂—O(C═O)—). The attachment of azido lipid (N₃-DO) was proved by the proton signals at δ 0.85 (m, 6H, —(CH₂)₆CH₃), 1.25 (m, 40H, —(CH₂)₄CH₂ CH═CHCH₂(CH₂)₆CH₃), 1.50 (m, 4H, —OC(═O)CH₂CH₂—), 1.98 (m, 8H, —CH₂CH═CHCH₂—), 2.35 (m, 4H, —OC(═O)CH₂—), 2.75 (m, 1H, CH[(CH₂)₃]), 4.01 (m, 4H, —CH[(CH₂)O-]2), 4.42 (m, 2H, triazole N—CH₂—), 5.32 (m, 4H, —CH₂CH═CHCH₂—), 7.89 (br s, 1H, triazole ring, resulted by ‘Click’ reaction). Additionally, one of the pendant group peaks appears at δ 1.81 (m, 2H, backbone-CH₂CH₂CH₂-triazole ring, due to the click). The number of pendant groups was determined from the ¹H NMR analysis to be 1.0 for the NMP moiety and 1.0 for the DO lipid moiety by comparing the integrations of pendant group protons and polymer backbone protons, and the MALDI-TOF gave Mn 1623 Da (Calcd Mn 1539 Da).

### Step 3B. Synthesis of DO-PEOZ 700-MPZ

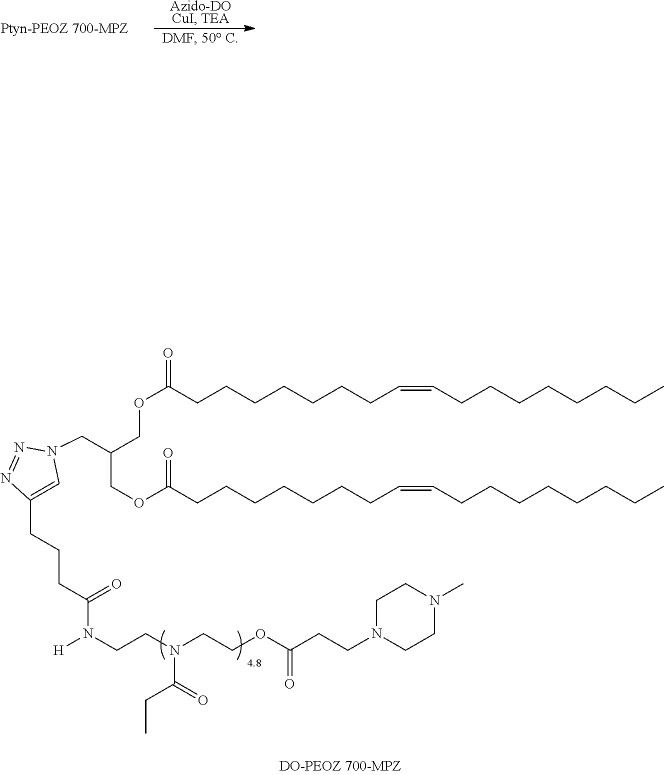

DO-PEOZ 700-MPZ was prepared in an analogous fashion to that described for DO-PPOZ 500-NMP using Ptyn-PEOZ 700-MPZ (54.5 mg, 0.0632 mmol, 1.0 equiv), 2-(azidomethyl)propane-1,3-diyl dioleate (azido-DO, 45.9 mg, 0.0695 mmol, 1.1 equiv), CuI (2.41 mg, 0.0126 mmol, 0.2 equiv), TEA (18.5 μL, 0.189 mmol, 3.0 eq), and DMF (2.0 mL) under an argon atmosphere. The crude product was purified by reverse-phase C18 column chromatography using water and methanol as the eluents, affording the title compound (11.0 mg, 11.0% yield).

Besides the standard polymer backbone peaks, ¹H NMR (Varian, 500 MHz, 10 mg/mL DMSO-d₆, δ) analysis showed signals for the N-methyl piperazinyl group were present at δ 2.30 (br s, 3H, PZ—N—CH₃), 2.40 (m, total area 8H, piperazinyl), 2.55 (m, 4H, —C(═O)CH₂CH₂N—PZ), 4.10 and 4.15 (br s, 2H, —CH₂—O(C═O)—). The attachment of azido lipid (N₃-DO) was proved by the proton signals at δ 0.85 (m, 6H, —(CH₂)₆CH₃), 1.24 (m, 40H, —(CH₂)₄CH₂ CH═CHCH₂(CH₂)₆CH₃), 1.50 (m, 4H, —OC(═O)CH₂CH₂—), 1.98 (m, 8H, —CH₂CH═CHCH₂—), 2.35 (m, 4H, —OC(═O)CH₂—), 2.75 (m, 1H, CH[(CH₂)₃]), 4.00 (m, 4H, —CH[(CH₂)O-]₂), 4.41 (m, 2H, triazole N—CH₂—), 5.32 (m, 4H, —CH₂CH═CHCH₂—), 7.88 (br s, 1H, triazole ring, resulted by ‘Click’ reaction). Additionally, one of the pendant group peaks appears at δ 1.80 (m, 2H, backbone-CH₂CH₂CH₂-triazole ring, due to the click). The number of pendant groups was determined from the ¹H NMR analysis to be 1.0 for the MPZ moiety and 1.0 for the DO lipid moiety by comparing the integrations of pendant group protons and polymer backbone protons, and the MALDI-TOF gave Mn 1480 Da (Calcd Mn 1523 Da).

### Step 3C. Synthesis of DO-PEOZ 700-DEA

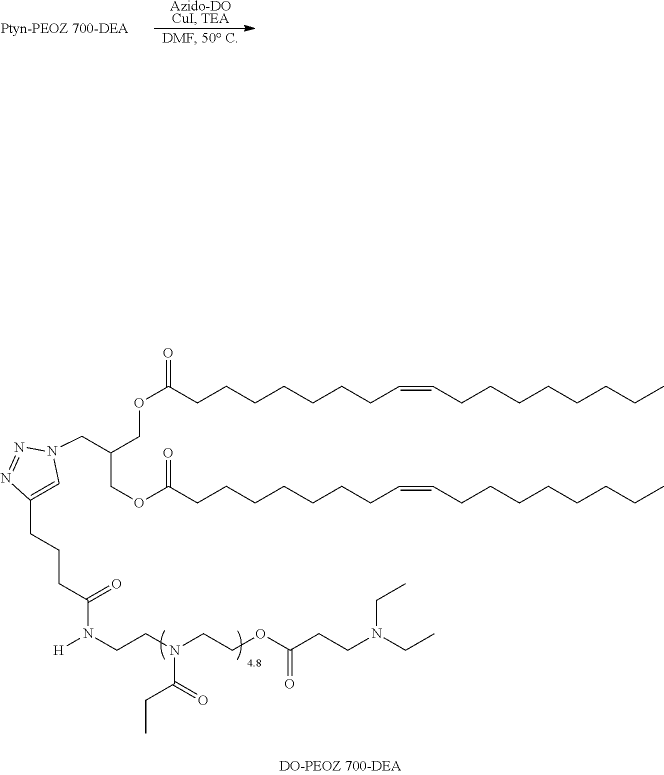

DO-PEOZ 700-DEA was prepared in an analogous fashion to that described for DO-PPOZ 500-NMP using Ptyn-PEOZ 700-DEA (50.0 mg, 0.0654 mmol, 1.0 equiv), 2-(azidomethyl)propane-1,3-diyl dioleate (azido-DO, 56.1 mg, 0.0850 mmol, 1.3 equiv), CuI (3.73 mg, 0.0196 mmol, 0.3 equiv), TEA (42.0 μL, 0.300 mmol, 4.6 eq), and DMF (2.0 mL) under an argon atmosphere. The crude product was purified by reverse-phase C18 column chromatography using water and methanol as the eluents, affording the title compound (55.2 mg, 59.3% yield).

Besides the standard polymer backbone peaks, ¹H NMR (Varian, 500 MHz, 10 mg/mL DMSO-d₆, δ) analysis showed signals for the diethylamino ethyl group were present at δ 1.19 (t, 6H, —N(CH₂CH₃)₂), 3.18 (m, 4H, —N(CH₂CH₃)₂), 4.16 (br s, 2H, —CH₂—O(C═O)—). The attachment of azido lipid (N₃-DO) was proved by the proton signals at δ 0.85 (m, 6H, —(CH₂)₆CH₃), 1.25 (m, 40H, —(CH₂)₄CH₂ CH═CHCH₂(CH₂)₆CH₃), 1.50 (m, 4H, —OC(═O)CH₂CH₂—), 1.98 (m, 8H, —CH₂CH═CHCH₂—), 2.61 (m, 4H, —OC(═O)CH₂—), 2.77 (m, 1H, CH[(CH₂)₃]), 4.01 (m, 4H, —CH[(CH₂)O-]₂), 4.41 (m, 2H, triazole N—CH₂—), 5.32 (m, 4H, —CH₂CH═CHCH₂—), 7.88 (br s, 1H, triazole ring, resulted by ‘Click’ reaction). Additionally, one of the pendant group peaks appears at δ 1.81 (m, 2H, backbone-CH₂CH₂CH₂-triazole ring, due to the click). The number of pendant groups was determined from the ¹H NMR analysis to be 1.0 for the DEA moiety and 1.0 for the DO lipid moiety by comparing the integrations of pendant group protons and polymer backbone protons, and the MALDI-TOF gave Mn 1404 Da (Calcd Mn 1425 Da).

### Example 9. Preparation of Lipid Nanoparticles Containing saRNA and DO-[PPOZ 500/PEOZ 700]-Ionizable Group (IG)

Using the DO-PPOZ 500-ionizable group (IG) or DO-PEOZ 700-IG in Example 7 (Step 3) or Example 8 (Step 3), LNPs were formed as described below.

The self-amplifying RNA (saRNA) was the Enhanced Green Fluorescent Protein encoded with the EGFP gene from the jellyfish, Aequorea victoria (Creative Biogene, PMSAR-0001). The single-stranded RNA has 8,528 nucleotides and the EGFP has a molecular weight of 27 kDa. The supplied solution of 1.687 μg/μL in water, was diluted with 20 mM sodium acetate buffer, pH 4.5, to prepare a solution of 0.01 μg/μL concentration.

Each lipid stock solution of DO-PPOZ 500-IG or DO-PEOZ 700-IG (from Example 7 (Steps 3A-3C) or Example 8 (Step 3A-3C)), 1′,3′-bis[1,2-dioleoyl-sn-glycero-3-phospho]-glycerol (18:1 Cardiolipin, Avanti 710335), cholesterol (VWR 0433) and DMA-PEOZ 2k (as described in U.S. Pat. No. 12,233,132, the entire disclosure of which is incorporated by reference herein) were prepared in ethanol (EMD Millipore EX0276-4). The stock solutions were mixed so that the concentration of lipid mixture was prepared as 2 mg/mL, with varying mol % of cholesterol and N/P ratios as shown in Table 4 below.

TABLE 4
saRNA-LNP systems containing DO-[PPOZ 500/PEOZ 700]-IG ¹

| | Ionizable lipid/18:1 CL/Cholesterol/ | |
|---|---|---|
| LNP # | DMA-PEOZ 2k mol % | N/P ratio |
| 1 | 25:10:63.5:1.5 | 6 |
| 2 | 50:10:38.5:1.5 | 10 |
| 3 | 70:10:18.5:1.5 | 12 |

¹ with 25 to 70 mol % at the expense of cholesterol from 63.5 to 18.5 mol % (keeping 18:1 CL and DMA-PEOZ 2k mol % constant)

The DO-[PPOZ 500/PEOZ 700]-IG in ethanol was mixed with saRNA in 10 mM Na Acetate, pH 4.5 at a ratio of three parts aqueous to one part organic using a pipette via hand mixing and then diluted 10 times with 25 mM Tris-HCl, pH 7.4. The resulting mixture was incubated for one hour at room temperature to reduce ethanol content in the lipid nanoparticles.

### Example 10. Characterization of saRNA-LNPs Containing DO-[PPOZ 500/PEOZ 700]-IG

Each lipid stock solution of DO-PPOZ 500-IG or DO-PEOZ 700-IG (from Step 3A-3C of Example 7 or from Step 3A-3C of Example 8), 1′,3′-bis[1,2-dioleoyl-sn-glycero-3-phospho]-glycerol (18:1 Cardiolipin, Avanti 710335), cholesterol (VWR 0433) and PEOZ 2k DMA (as described in U.S. Pat. No. 12,233,132) were prepared in ethanol (EMD Millipore EX0276-4). The stock solutions were mixed so that the concentration of lipid mixture was prepared as 2 mg/mL, with varying mol % of cholesterol.

Selected molar ratios for four lipid mixture, DO-[PPOZ 500/PEOZ 700]-IG/18:1 CL/Cholesterol/DMA-PEOZ 2k mol %, were 70:10:18.5:1.5, respectively. The lipids in ethanol was mixed with saRNA in 10 mM Na Acetate, pH 4.5 at a ratio of three parts aqueous to one part organic using a pipette via hand mixing and then diluted 10 times with 25 mM Tris-HCl, pH 7.4. The resulting mixture was incubated for one hour at room temperature to reduce ethanol content in the lipid nanoparticles.

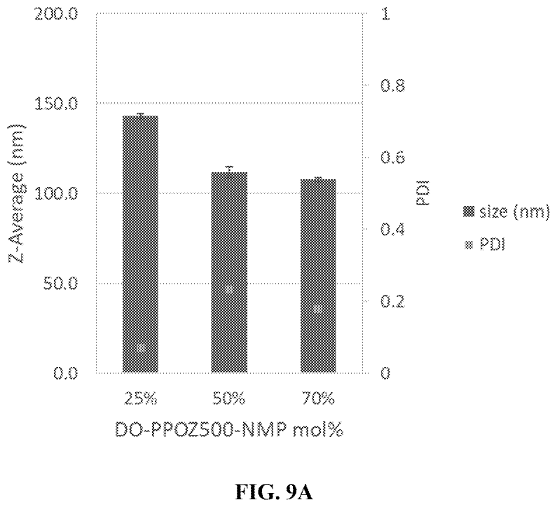

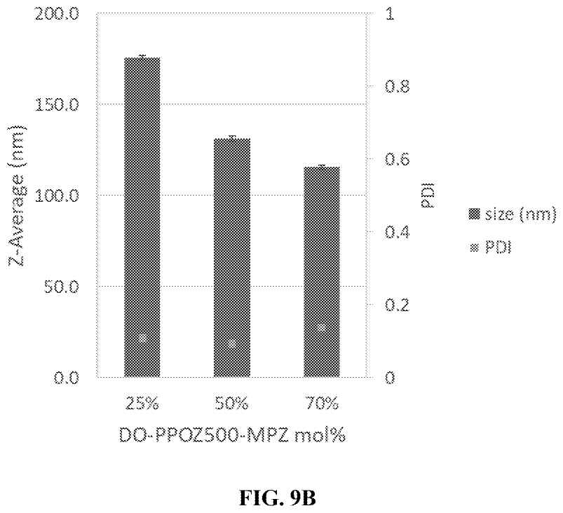

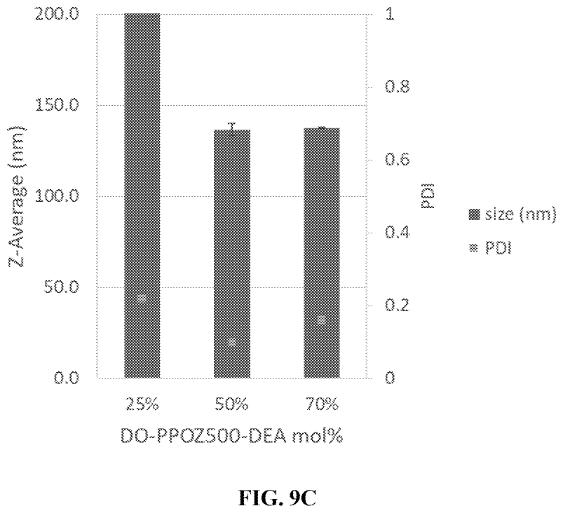

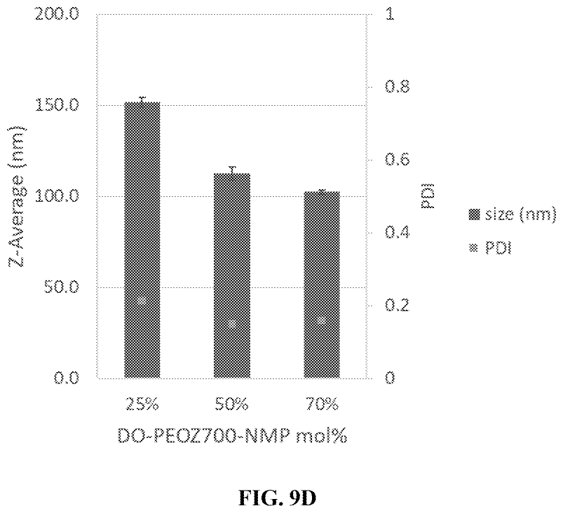

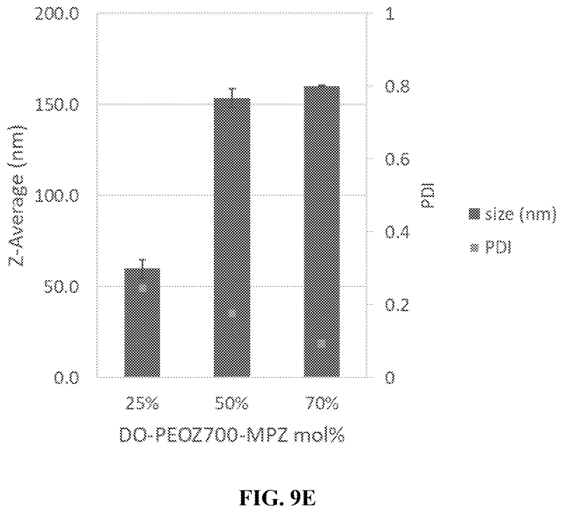

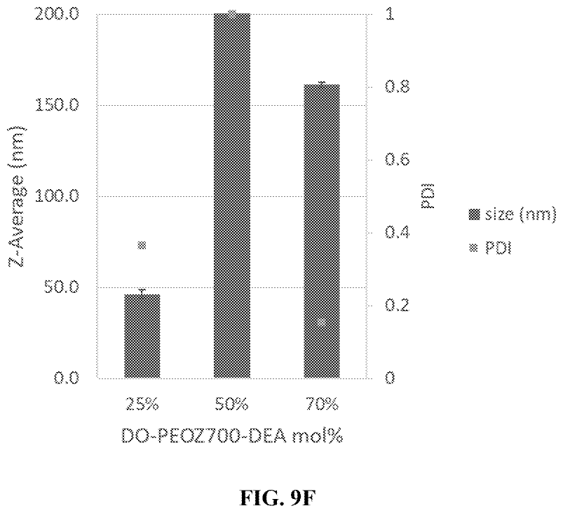

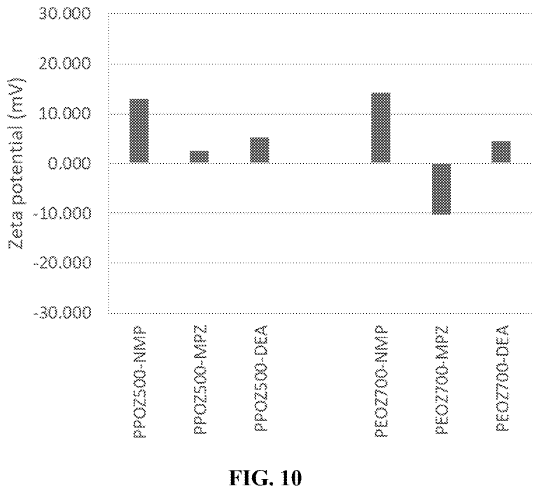

The size of the formulated saRNA-LNPs was measured using dynamic light scattering (DLS) (Zetasizer Ultra, Malvern Panalytical). FIGS. 9A-9F shows the particle size of the LNPs averaged after three measurements ranged between 100 to 150 nm with low PDI (<0.2) at a range of 50-70 mol % DO-[PPOZ 500/PEOZ 700]-IG. The zeta potential of the saRNA-LNPs was measured using a Zetasizer Ultra (Malvern Panalytical) with electrophoretic light scattering capabilities. The saRNA-LNPs were diluted nine times with 25 mM Tris-HCl, pH 7.5, buffer to measure the zeta potential. FIG. 10 shows the zeta potential recorded after measurements.

TABLE 5
Characterization of saRNA-LNPs
with DO-[PPOZ 500/PEOZ 700]-IG

| | Size (nm) | PDI | Zeta potential (mV) |
|---|---|---|---|
| DO-PPOZ 500-NMP | 100.7 | 0.178 | 13.0 |
| DO-PPOZ 500-MPZ | 103.3 | 0.142 | 2.5 |
| DO-PPOZ 500-DEA | 110.4 | 0.205 | 5.3 |
| DO-PEOZ 700-NMP | 100.7 | 0.137 | 14.1 |
| DO-PEOZ 700-MPZ | 166.6 | 0.100 | −10.3 |
| DO-PEOZ 700-DEA | 150.4 | 0.107 | 4.5 |

### Example 11. In Vitro Transfection of HEK293 Cells with FLuc saRNA LNP Containing DO-PPOZ 500/PEOZ 700-IG

The self-amplifying RNA (saRNA) was the luciferase protein encoded with the luciferase gene from the firefly, Photinus pyralis (Creative Biogene, PMSAR-0002). The single-stranded RNA has 9,461 nucleotides, and the firefly luciferase has a molecular weight of 62 kDa. The supplied solution of 1.109 μg/μL in water, was diluted with 10 mM sodium acetate buffer, pH 4.5, to prepare a solution of 0.01 μg/μL concentration.

LNP lipid stocks were prepared in ethanol at selected molar ratios of DO-PPOZ 500-IG or DO-PEOZ 700-IG, 1′,3′-bis[1,2-dioleoyl-sn-glycero-3-phospho]-glycerol (18:1 Cardiolipin, Avanti 710335), cholesterol (VWR 0433) and PEOZ 2k DMA (as described in U.S. Pat. No. 12,233,132) in EtOH at a lipid stock concentration of 2 mg/mL. The saRNA aqueous stock was hand-mixed with the lipid stocks at a 3:1 ratio and diluting to a 0.75 ng/μL saRNA concentration with 25 mM Tris, pH 7.5. The resulting mixture was incubated for one hour at room temperature to reduce ethanol content in the lipid nanoparticles.

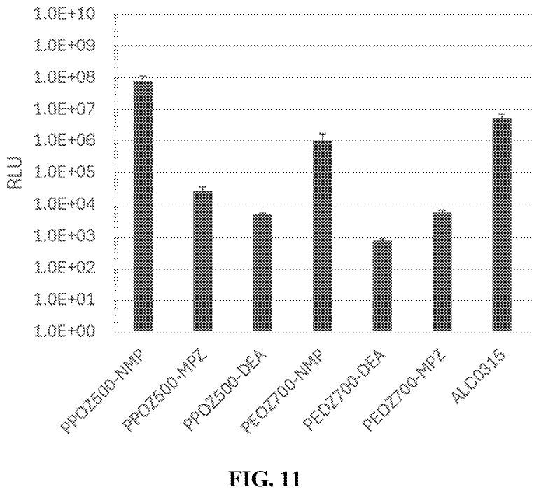

Human Embryonic Kidney (HEK) 293 cells (ATCC) were seeded in a 96 well cell treated plate (Corning) at a density of 25,000 cells/well in 100 μL of DMEM media (Corning) with 10% FBS (Corning). The HEK293 plate was incubated at 37° C./5% CO2 overnight to a confluency of about 50 to 60%. Following incubation, each well was treated with LNPs at a 50 ng FLuc saRNA concentration. Media was removed from each well, and replaced with 100 μL Opti-MEM, reduced serum medium (Gibco). The plate was returned to the incubator to allow for cellular uptake of the formulations. After 3 to 4 hours, the wells containing Opti-MEM were replaced with 100 μL DMEM with 10% FBS and the plate was returned to incubator for 24 hours. Media was removed from each well and replaced with 100 μL of luciferase reagent (Promega, Bright-Glo™ Luciferase Assay System). The plate was left to incubate for 5 min at room temperature. The luminescence of the plates was read using a Varioskan LUX (Invitrogen) (FIG. 11).

### Example 12. Cellular Uptake of FITC-Labeled RNA-LNPs Containing DO-[PPOZ 500/PEOZ 700]-NMP

LNP lipid stocks were prepared in ethanol at selected molar ratios of DO-PPOZ 500-NMP or DO-PEOZ 700-NMP, 1′,3′-bis[1,2-dioleoyl-sn-glycero-3-phospho]-glycerol (18:1 Cardiolipin, Avanti 710335), cholesterol (VWR 0433) and PEOZ 2k DMA (as described in U.S. Pat. No. 12,233,132) in EtOH at a lipid stock concentration of 2 mg/mL. The RNA aqueous stock was prepared by mixing poly A (Millipore Sigma, 10108626001) and FITC-siRNA (Cell Signaling Technology, 6201S) at a weight ratio of 9:1, diluting with 10 mM sodium acetate buffer, pH 4.5 to prepare a solution of 0.01 μg/μL concentration. The FITC-labeled RNA stock solution was hand-mixed with the lipid stocks at a 3:1 ratio and then diluted 10 times with 25 mM Tris-HCl, pH 7.4. The resulting mixture was incubated for one hour at room temperature to reduce ethanol content in the lipid nanoparticles.

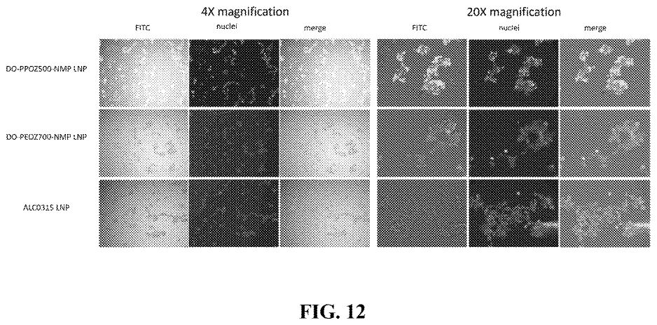

Human Embryonic Kidney (HEK) 293 cells (ATCC) were seeded in a 96 well cell treated plate (Corning) at a density of 25,000 cells/well in 100 μL of DMEM media (Corning) with 10% FBS (Corning). The HEK293 plate was incubated at 37° C./5% CO2 overnight to a confluency of about 50 to 60%. Following incubation, each well was treated with LNPs at a 100 nM FITC-RNA concentration. After transfection for 24 hours, cell nuclei were stained with Hoechst 33258. Then cells were analyzed on an EVOS-M7000 microscope (Thermoscientific) for cellular uptake of LNP. As demonstrated in FIG. 12, the transfection of FITC-RNA LNPs yielded a positive green signal indicating cellular uptake of LNPs.

### Example 13. In Vitro Transfection of HEK293 Cells with FLuc saRNA LNP Containing DO-[PPOZ 500/PEOZ 700]-NMP

The self-amplifying RNA (saRNA) was the luciferase protein encoded with the luciferase gene from the firefly, Photinus pyralis (Creative Biogene, PMSAR-0002). The single-stranded RNA has 9,461 nucleotides, and the firefly luciferase has a molecular weight of 62 kDa. The supplied solution of 1.109 μg/μL in water, was diluted with 10 mM sodium acetate buffer, pH 4.5, to prepare a solution of 0.01 μg/μL concentration.

LNP lipid stocks were prepared in ethanol with varying molar ratios of DO-PPOZ 500-NMP or DO-PEOZ 700-NMP, 1′,3′-bis[1,2-dioleoyl-sn-glycero-3-phospho]-glycerol (18:1 Cardiolipin, Avanti 710335), cholesterol (VWR 0433) and PEOZ 2k DMA (as described in U.S. Pat. No. 12,233,132) in EtOH at a lipid stock concentration of 2 mg/mL. The saRNA aqueous stock was hand-mixed with the lipid stocks at a 3:1 ratio and diluting to a 0.75 ng/μL saRNA concentration with 25 mM Tris, pH 7.5. The resulting mixture was incubated for one hour at room temperature to reduce ethanol content in the lipid nanoparticles. After initial screening, LNP lipid stocks were prepared in ethanol at selected molar ratios of DO-PPOZ 500-IG or DO-PEOZ 700-IG, 1′,3′-bis[1,2-dioleoyl-sn-glycero-3-phospho]-glycerol (18:1 Cardiolipin, Avanti 710335), cholesterol (VWR 0433) and PEOZ 2k DMA (as described in U.S. Pat. No. 12,233,132) and mixed with the Fluc saRNA to formulate LNPs containing DO-PPOZ 500-NMP or DO-PEOZ 700-NMP.

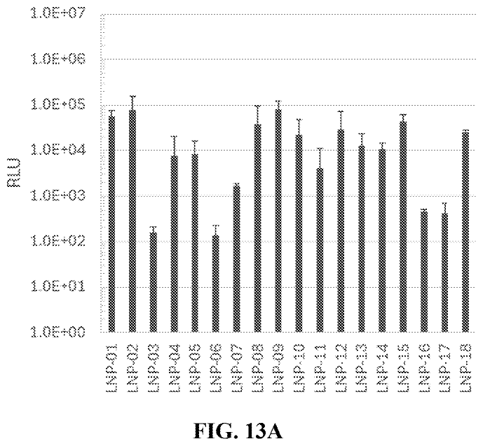

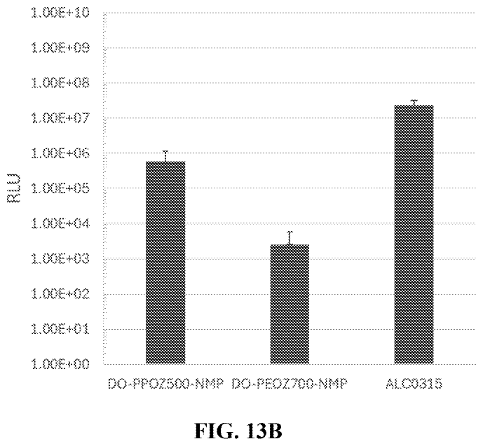

Human Embryonic Kidney (HEK) 293 cells (ATCC) were seeded in a 96 well cell treated plate (Corning) at a density of 25,000 cells/well in 100 μL of DMEM media (Corning) with 10% FBS (Corning). The HEK293 plate was incubated at 37° C./5% CO2 overnight to a confluency of about 50 to 60%. Following incubation, each well was treated with LNPs at a 50 ng FLuc saRNA concentration. After transfection for 24 hours, media was removed from each well and replaced with 100 μL of luciferase reagent (Promega, Bright-Glo™ Luciferase Assay System). The plate was left to incubate for 5 min at room temperature. The luminescence of the plates was read using a Varioskan LUX (Invitrogen) (FIGS. 13A-B).

The cationic POZ-lipids, LNPs and polyplexes, and pharmaceutical compositions described and claimed herein are not to be limited in scope by the specific embodiments herein disclosed, since these embodiments are intended as illustrations of several aspects of the disclosure. Any equivalent embodiments are intended to be within the scope of this disclosure. Indeed, various modifications of the formulas and structures in addition to those shown and described herein will become apparent to those skilled in the art from the foregoing description. Such modifications are also intended to fall within the scope of the appended claims. All patents and patent applications cited in the foregoing text are expressly incorporated herein by reference in their entirety. Any section headings herein are provided only for consistency with the suggestions of 37 C.F.R. § 1.77 or otherwise to provide organizational queues. These headings shall not limit or characterize the invention(s) set forth herein.

What is claimed is:

1. A lipid nanoparticle comprising:
a cationic POZ-lipid;
a helper lipid;
a polymer lipid; and
a sterol lipid, wherein the cationic POZ-lipid is a cationic POZ-lipid of Formula I: 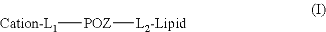
wherein L₁ comprises a first linking group,
POZ comprises a polyoxazoline polymer,
L₂ comprises a second linking group, and
Lipid comprises a non-charged lipid.

2. The lipid nanoparticle of claim 1, wherein the lipid nanoparticle comprises about 15 percent to about 60 percent cationic POZ-lipid, about 35 percent to about 70 percent sterol lipid, about 0.5 percent to about 10 percent polymer lipid, and about 5 percent to about 20 percent helper lipid.

3. The lipid nanoparticle of claim 1, wherein the lipid nanoparticle comprises about 5 percent to about 60 percent cationic POZ-lipid, about 30 percent to about 85 percent sterol lipid, about 0.5 percent to about 10 percent polymer lipid, and about 5 percent to about 20 percent helper lipid.

4. The cationic POZ-lipid of claim 1, wherein the first and second linking groups are different.

5. The lipid nanoparticle of claim 1, wherein the cationic POZ-lipid is a cationic POZ-lipid of Formula I(a): 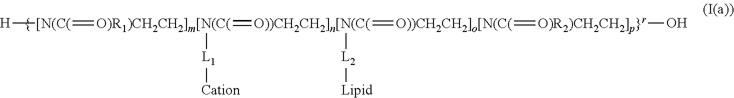
wherein R₁ is independently selected for each repeating unit from an unsubstituted or substituted alkyl,
m ranges from 0 to 25,
n ranges from 1 to 4,
o ranges from 1 to 4,
R₂ is independently selected for each repeating unit from an unsubstituted or substituted alkynyl,
p ranges from 0 to 3, and
r is random.

6. The lipid nanoparticle of claim 5, wherein R₁ is propyl.

7. The lipid nanoparticle of claim 5, wherein R₂ is pentynyl.

8. The lipid nanoparticle of claim 5, wherein n+o+p may range from 2 to 5.

9. The cationic POZ-lipid of claim 5, wherein the first and second linking groups are different.

10. A lipid nanoparticle comprising:
a cationic POZ-lipid;
a helper lipid;
a polymer lipid; and
a sterol lipid, wherein the cationic POZ-lipid is a cationic POZ-lipid of Formula II: 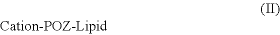
wherein POZ comprises a polyoxazoline polymer, and
Lipid comprises a non-charged lipid.

11. The lipid nanoparticle of claim 10, wherein the cationic POZ-lipid is a cationic POZ-lipid of Formula II(a): 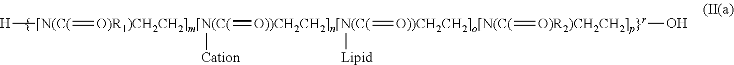
wherein R₁ is independently selected for each repeating unit from an unsubstituted or substituted alkyl,
m ranges from 0 to 25,
n ranges from 1 to 4,
o ranges from 1 to 4,
R₂ is independently selected for each repeating unit from an unsubstituted or substituted alkynyl,
p ranges from 0 to 3, and
r is random.

12. The lipid nanoparticle of claim 11, wherein R₁ is propyl.

13. The lipid nanoparticle of claim 11, wherein R₂ is pentynyl.

14. The lipid nanoparticle of claim 11, wherein n+o+p may range from 2 to 5.

15. The lipid nanoparticle of claim 10, wherein the lipid nanoparticle comprises about 15 percent to about 60 percent cationic POZ-lipid, about 35 percent to about 70 percent sterol lipid, about 0.5 percent to about 10 percent polymer lipid, and about 5 percent to about 20 percent helper lipid.

16. The lipid nanoparticle of claim 10, wherein the lipid nanoparticle comprises about 5 percent to about 60 percent cationic POZ-lipid, about 30 percent to about 85 percent sterol lipid, about 0.5 percent to about 10 percent polymer lipid, and about 5 percent to about 20 percent helper lipid.

17. The lipid nanoparticle of claim 10, wherein the cationic POZ-lipid is a cationic POZ-lipid of Formula II(b): 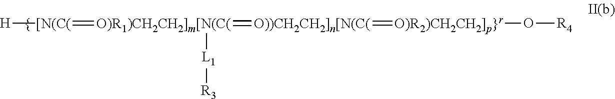
wherein R₁ is independently selected for each repeating unit from an unsubstituted or substituted alkyl,
R₃ is Cation,
m ranges from 0 to 25,
L₁ comprises a first linking group,
n ranges from 1 to 5,
R₂ is independently selected for each repeating unit from an unsubstituted or substituted alkynyl,
p ranges from 0 to 4,
r is random, and
R₄ is Lipid.

18. The lipid nanoparticle of claim 17, wherein R₁ is ethyl, propyl, pentynyl, propynyl, butynyl, or acetylenyl.

19. The lipid nanoparticle of claim 17, wherein R₂ is 2-pentynyl or 4-pentynyl.

20. The lipid nanoparticle of claim 17, wherein n+p may range from 1 to 5.

21. The lipid nanoparticle of claim 10, wherein the cationic POZ-lipid is a cationic POZ-lipid of Formula II(c): 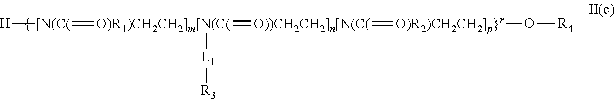
where R₁ is independently selected for each repeating unit from an unsubstituted or substituted alkyl,
R₃ is Lipid,
m ranges from 0 to 25,
L₁ comprises a first linking group,
n ranges from 1 to 5,
R₂ is independently selected for each repeating unit from an unsubstituted or substituted alkynyl,
p ranges from 0 to 4,
r is random, and
R₄ is Cation.

22. The lipid nanoparticle of claim 21, wherein R₁ is ethyl or propyl.

23. The lipid nanoparticle of claim 21, wherein R₂ is pentynyl.

24. The lipid nanoparticle of claim 21, wherein n+p may range from 1 to 5.

25. The lipid nanoparticle of claim 10, wherein the cationic POZ-lipid is a cationic POZ-lipid of Formula II(d): 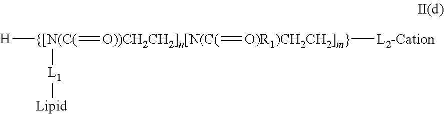
wherein R₁ is independently selected for each repeating unit from an unsubstituted or substituted alkyl,
n ranges from 1 to 2,
L₁ comprises a first linking group,
m ranges from 1 to 25,
L₂ comprises a second linking group.

26. The lipid nanoparticle of claim 25, wherein R₁ is ethyl, propyl, pentynyl, propynyl, butynyl, or acetylenyl.

27. The lipid nanoparticle of claim 25, wherein n+m ranges from 2 to 6.

28. The lipid nanoparticle of claim 10, wherein the cationic POZ-lipid is a cationic POZ-lipid of Formula II(e): 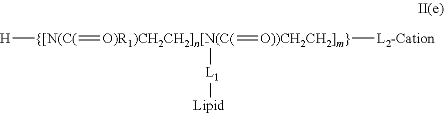
wherein R₁ is independently selected for each repeating unit from an unsubstituted or substituted alkyl,
n ranges from 1 to 25,
L₁ comprises a first linking group,
m ranges from 1 to 2,
L₂ comprises a second linking group.

29. The lipid nanoparticle of claim 28, wherein R₁ is ethyl, propyl, pentynyl, propynyl, butynyl, or acetylenyl.

30. The lipid nanoparticle of claim 28, wherein n+m ranges from 2 to 6.

31. A lipid nanoparticle comprising:
a cationic POZ-lipid;
a helper lipid;
a polymer lipid; and
a sterol lipid, wherein the cationic POZ-lipid is selected from the group consisting of a cationic POZ-lipid of Formulas II(d) and II(e): 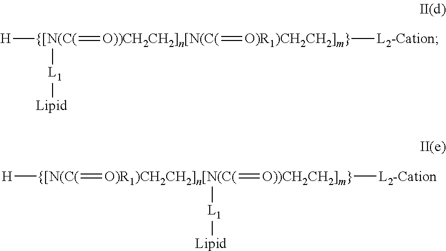
wherein R₁ is independently selected for each repeating unit from R1 is ethyl, propyl, pentynyl, propynyl, butynyl, or acetylenyl,
n+m ranges from 1 to 20,
L₁ comprises a first linking group,
L₂ comprises a second linking group, and
Lipid comprises a non-charged lipid.
